SEQUENCE LISTING (20 sequences)

SEQ ID NO: 1 - protein, 204 aa, Homo sapiens
HTDLSGKVFVFPRESVTDHVNLITPLEKPLQNFTLCFRAYSDLSRAYSLFSYNTQGRDNELLVYKERVGEYSLYIGRHKVTSKVIEKFPAPVHICVSWESSSGIAEFWINGTPLVKKGLRQGYFVEAQPKIVLGQEQDSYGGKFDRSQSFVGEIGDLYMWDSVLPPENILSAYQGTPLPANILDWQALNYEIRGYVIIKPLVWV

SEQ ID NO: 2 - protein, 208 aa, Gallus gallus
QEDLYRKVFVFREDPSDAYVLLQVQLERPLLNFTVCLRSYTDLTRPHSLFSYATKAQDNEILLFKPKPGEYRFYVGGKYVTFRVPENRGEWEHVCASWESGSGIAEFWLNGRPWPRKGLQKGYEVGNEAVVMLGQEQDAYGGGFDVYNSFTGEMADVHLWDAGLSPDKMRSAYLALRLPPAPLAWGRLRYEAKGDVVVKPRLREALGA

SEQ ID NO: 3 - protein, 205 aa, Bos taurus
QTDLRGKVFVFPRESSTDHVTLITKLEKPLKNLTLCLRAYSDLSRGYSLFSYNIHSKDNELLVFKNGIGEYSLYIGKTKVTVRATEKFPSPVHICTSWESSTGIAEFWINGKPLVKRGLKQGYAVGAHPKIVLGQEQDSYGGGFDKNQSFMGEIGDLYMWDSVLSPEEILLVYQGSSSISPTILDWQALKYEIKGYVIVKPMVWG

SEQ ID NO: 4 - protein, 204 aa, Cricetulus migratorius
QTDLTGKVFVFPRESESDYVKLIPRLEKPLENFTLCFRTYTDLSRPHSLFSYNTKNKDNELLIYKERMGEYGLYIENVGAIVRGVEEFASPVHFCTSWESSSGIADFWVNGIPWVKKGLKKGYTVKTQPSIILGQEQDNYGGGFDKSQSFVGEMGDLNMWDSVLTPEEIKSVYEGSWLEPNILDWRALNYEMSGYAVIRPRVWH

SEQ ID NO: 5 - DNA, 5284 bp, Homo sapiens
ctgcaggaaggagagaggaagaggagcagaagggggcagcagcggacgccgctaacggcctccctcggcgctgacaggctgggccggcgcccggctcgcttgggtgttcgcgtcgccacttcggcttctcggccggtcgggcccctcggcccgggcttgcggcgcgcgtcggggctgagggctgctgcggcgcagggagaggcctggtcctcgctgccgagggatgtgagtgggagctgagcccacactggagggcccccgagggcccagcctggaggtcgttcagagccgtgcccgtcccggggcttcgcagaccttgacccgccgggtaggagccgcccctgcgggctcgagggcgcgctctggtcgcccgatctgtgtagccggtttcagaagcaggcaacaggaacaagatgtgaactgtttctcttctgcagaaaaagaggctcttcctcctcctcccgcgacggcaaatgttctgaaaaagactctgcatgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctggggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatgaaagaaatgaccttcattctgagaccaaagtagatttacagaacaaagttttatatttcacattgctgtggactattattacatatatcattattatataaatcatgatgctagccagcaaagatgtgaaaatatctgctcaaaactttcaaagtttagtaagtttttcttcatgaggccaccagtaaaagacattaatgagaattccttagcaaggattttgtaagaagtttcttaaacattgtcagttaacatcactcttgtctggcaaaagaaaaaaaatagactttttcaactcagctttttgagacctgaaaaaattattatgtaaattttgcaatgttaaagatgcacagaatatgtatgtatagtttttaccacagtggatgtataataccttggcatcttgtgtgatgttttacacacatgagggctggtgttcattaatactgttttctaatttttccatagttaatctataattaattacttcactatacaaacaaattaagatgttcagataattgaataagtacctttgtgtccttgttcatttatatcgctggccagcattataagcaggtgtatacttttagcttgtagttccatgtactgtaaatatttttcacataaagggaacaaatgtctagttttatttgtataggaaatttccctgaccctaaataatacattttgaaatgaaacaagcttacaaagatataatctattttattatggtttcccttgtatctatttgtggtgaatgtgttttttaaatggaactatctccaaatttttctaagactactatgaacagttttcttttaaaattttgagattaagaatgccaggaatattgtcatcctttgagctgctgactgccaataacattcttcgatctctgggatttatgctcatgaactaaatttaagcttaagccataaaatagattagattgttttttaaaaatggatagctcattaagaagtgcagcaggttaagaattttttcctaaagactgtatatttgaggggtttcagaattttgcattgcagtcatagaagagatttatttcctttttagaggggaaatgaggtaaataagtaaaaaagtatgcttgttaattttattcaagaatgccagtagaaaattcataacgtgtatctttaagaaaaatgagcatacatcttaaatcttttcaattaagtataaggggttgttcgttgttgtcatttgttatagtgctactccactttagacaccatagctaaaataaaatatggtgggttttgtgtgtgtgtgtgtgtgtgtgtgtgtgtgtgtgtgtgtgttatttatacaaaacttaaaatacttgctgttttgattaaaaagaaaatagtttcttacttt

SEQ ID NO: 6 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 7 - DNA, 3645 bp, Homo sapiens
cctgaagggaggatgggctaaggcaggcacacagtggcggagaagatgccctcctgggccctcttcatggtcacctcctgcctcctcctggcccctcaaaacctggcccaagtcagcagccaagatgtctccttgctggcatcagactcagagcccctgaagtgtttctcccgaacatttgaggacctcacttgcttctgggatgaggaagaggcagcgcccagtgggacataccagctgctgtatgcctacccgcgggagaagccccgtgcttgccccctgagttcccagagcatgccccactttggaacccgatacgtgtgccagtttccagaccaggaggaagtgcgtctcttctttccgctgcacctctgggtgaagaatgtgttcctaaaccagactcggactcagcgagtcctctttgtggacagtgtaggcctgccggctccccccagtatcatcaaggccatgggtgggagccagccaggggaacttcagatcagctgggaggagccagctccagaaatcagtgatttcctgaggtacgaactccgctatggccccagagatcccaagaactccactggtcccacggtcatacagctgattgccacagaaacctgctgccctgctctgcagaggcctcactcagcctctgctctggaccagtctccatgtgctcagcccacaatgccctggcaagatggaccaaagcagacctccccaagtagagaagcttcagctctgacagcagagggtggaagctgcctcatctcaggactccagcctggcaactcctactggctgcagctgcgcagcgaacctgatgggatctccctcggtggctcctggggatcctggtccctccctgtgactgtggacctgcctggagatgcagtggcacttggactgcaatgctttaccttggacctgaagaatgttacctgtcaatggcagcaacaggaccatgctagctcccaaggcttcttctaccacagcagggcacggtgctgccccagagacaggtaccccatctgggagaactgcgaagaggaagagaaaacaaatccaggactacagaccccacagttctctcgctgccacttcaagtcacgaaatgacagcattattcacatccttgtggaggtgaccacagccccgggtactgttcacagctacctgggctcccctttctggatccaccaggctgtgcgcctccccaccccaaacttgcactggagggagatctccagtgggcatctggaattggagtggcagcacccatcgtcctgggcagcccaagagacctgttatcaactccgatacacaggagaaggccatcaggactggaaggtgctggagccgcctctcggggcccgaggagggaccctggagctgcgcccgcgatctcgctaccgtttacagctgcgcgccaggctcaacggccccacctaccaaggtccctggagctcgtggtcggacccaactagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttgaggacaggctcctcactcccagttccctggacagagctaaactctcgagacttctctgtgaacttccctaccctacccccacaacacaagcaccccagacctcacctccatccccctctgtctgccctcacaattaggcttcattgcactgatcttactctactgctgctgacataaaaccaggaccctttctccacaggcaggctcatttcactaagctcctcctttactttctctctcctctttgatgtcaaacgccttgaaaacaagcctccacttccccacacttcccatttactcttgagactacttcaattagttcccctactacactttgctagtgaaactgcccaggcaaagtgcacctcaaatcttctaattccaagatccaataggatctcgttaatcatcagttcctttgatctcgctgtaagatttgtcaaggctgactactcacttctcctttaaattctttcctaccttggtcctgcctctttgagtatattagtaggttttttttatttgtttgagacagggtctcactctgtcacccaggctgcagtgcaatggcgcgatctcagctcactgcaacctccacctccgggttcaagcgattcttgtgcctcggcctccctagtagctgggattacaggcgcacaccaccacacacagctaattttttttttttttttttttttttttttttagacggagccttgctctgttgccagactggagtgcagtggcacgatctcggctcactgcaacctctgcctcccgggttcaagccattctgcctcagcctcccaagtagctgggagtacaggcgtctgccaccatgcctaatttttttctatttttaggagagaccggttttcaccacgttggccaggatggtctcgatatcctgatctcgtgatccgcctgcctctgcctcccaaagtgctgggattacaggtgtgacccactgcgcacagccccagctaattttcatatttttagtagagacagggttttgccatgttgcccaggctggtcttgaactcctaacctcgggtgatccacccaccttggcctcccaaagtgttaggattacaggcatgagccactgcgcccggctgagtgtactagtagttaagagaataaactagatctagaatcagagctggattcaattcctgtccttcacatttactagctgtgcaaccttgggcacataacttaatgtctttgagccttagttttttcatctgtaaaacagggataataacagcaccccatagagttgtgacgaggattgagataatctaagtaaagcacagtccctaggacatagtaaatgattcatatatccgaactactgttataattattccttcttactctcctcttctagcatttcttccaattattacagtccttcaagattccatttcttaacagtctccaatcccatctattctctgcctttactatatgttgaccattccaaagttcttatctctagctcagacatctactacagcactgtgatgctttatgcaactaactgtttacatatctgtcccctgctactagattgtgagctccttgagggaaaggaacatgatttatttgtccttttcccccagcacctagagtagtgcttggtgcatgatagtaggccttcaataaattttttctaaatgaatga

SEQ ID NO: 8 - protein, 635 aa, Homo sapiens
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 9 - DNA, 1929 bp, Homo sapiens
gcggcgtccgtccgtactgcagagccgctgccggagggtcgttttaaagggcccgcgcgttgccgccccctcggcccgccatgctgctatccgtgccgctgctgctcggcctcctcggcctggccgtcgccgagcctgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctccaaaaaaaaaaaaaaaaaa

SEQ ID NO: 10 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 11 - DNA, 1084 bp, Homo sapiens
cacacccacggcagacacgcacgcacccgggcgccgaagggaaagccgcgtctcgccctcccgccccgccgtcggtcctgtctcagtccctcagcagagcgggaaagcggaggccggagccgtgacctctgaccccgtggttatgcggagccgccgcattccttagcgatcgcggggcagccgccgctgccgccgtgggcgactgacgcagcgcgggcgcgtggagccgccgccgcccctcccccaccgccgctctcgcgccagccggtccccgcgtgcccgccccttctccccggccgcacccgagacctcgcgcgccgccgctgccacgcgccccccccaccgccgccgccgccccagccccgcgccaccgccccagcccgcccagcccggaggtcccgcgtggagctgccgccgccgccggggagaaggatgaaggacaaacagaagaagaagaaggagcgcacgtgggccgaggccgcgcgcctggtattagaaaactactcggatgctccaatgacaccaaaacagattctgcaggtcatagaggcagaaggactaaaggaaatgagaagtgggacttcccctctcgcatgcctcaatgctatgctacattccaattcaagaggaggagaggggttgttttataaactgcctggccgaatcagccttttcacgctcaaggtgtgagccactgcaccaggccccttcatcttaattttaatatatctttgaataaacaccattgtatgaacctgctgtaagcttgggagtggtctgttagtctacagcttgtgtctgagatgtgctaattgaatatttgctcagtacctcatcttaactgcctttggctttatgttgcttatccttcatagtatcttgttcattggccttttacatccataggcatcacttctctgatattcgttgtgctcttttaatggattaatggtttgcttggttggttcctctagttagactgtaaactccttgagagcagagtctgtattttattaattacccacagtactaggtacatagttgccttcaataaatatatatttaatgaaaaaaaaaaaaaaaa

SEQ ID NO: 12 - protein, 85 aa, Homo sapiens
MKDKQKKKKERTWAEAARLVLENYSDAPMTPKQILQVIEAEGLKEMRSGTSPLACLNAMLHSNSRGGEGLFYKLPGRISLFTLKV

SEQ ID NO: 13 - DNA, 2708 bp, Homo sapiens
ggcggcgcttgattgggctgggggggccaaataaaagcgatggcgattgggctgccgcgtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtccttgttgaatc

SEQ ID NO: 14 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 15 - DNA, 2008 bp, Homo sapiens
agaaggtttcatttccgggtggcgcgggcgccattttgtgaggagcgatataaacgggcgcagaggccggctgcccgcccagttgttactcaggtgcgctagcctgcggagcccgtccgtgctgttctgcggcaaggcctttcccagtgtccccacgcggaaggcaactgcctgagaggcgcggcgtcgcaccgcccagagctgaggaagccggcgccagttcgcggggctccgggccgccactcagagctatgagctacggccgcccccctcccgatgtggagggtatgacctccctcaaggtggacaacctgacctaccgcacctcgcccgacacgctgaggcgcgtcttcgagaagtacgggcgcgtcggcgacgtgtacatcccgcgggaccgctacaccaaggagtcccgcggcttcgccttcgttcgctttcacgacaagcgcgacgctgaggacgctatggatgccatggacggggccgtgctggacggccgcgagctgcgggtgcaaatggcgcgctacggccgccccccggactcacaccacagccgccggggaccgccaccccgcaggtacgggggcggtggctacggacgccggagccgcagccctaggcggcgtcgccgcagccgatcccggagtcggagccgttccaggtctcgcagccgatctcgctacagccgctcgaagtctcggtcccgcactcgttctcgatctcggtcgacctccaagtccagatccgcacgaaggtccaagtccaagtcctcgtcggtctccagatctcgttcgcggtccaggtcccggtctcggtccaggagtcctcccccagtgtccaagagggaatccaaatccaggtcgcgatcgaagagtccccccaagtctcctgaagaggaaggagcggtgtcctcttaagaaaatgatgtatcggcaagcagtgtaaacggaggacttggggaaaaaggaccacatagtccatcgaagaagagtccttggaacaagcaactggctattgaaaaggttattttgtaacatttgtctaactttttacttgtttaagctttgcctcagttggcaaacttcattttatgtgccattttgttgctgttattcaaatttcttgtaatttagtgaggtgaacgacttcagatttcattattggatttggatatttgaggtaaaatttcattttgttatatagtgctgactttttttgtttgaaattaaacagattggtaacctaatttgtggcctcctgacttttaaggaaaacgtgtgcagccattacacacagcctaaagctgtcaagagattgactcggcattgccttcattccttaaaattaaaaacctacaaaagttggtgtaaatttgtatatgttatttaccttcagatctaaatggtaatctgaacccaaatttgtataaagacttttcaggtgaaaagacttgattttttgaaaggattgtttatcaaacacaattctaatctcttctcttatgtatttttgtgcactaggcgcagttgtgtagcagttgagtaatgctggttagctgttaaggtggcgtgttgcagtgcagagtgcttggctgtttcctgttttctcccgattgctcctgtgtaaagatgccttgtcgtgcagaaacaaatggctgtccagtttattaaaatgcctgacaactgcacttccagtcacccgggccttgcatataaataacggagcatacagtgagcacatctagctgatgataaatacacctttttttccctcttccccctaaaaatggtaaatctgatcatatctacatgtatgaacttaacatggaaaatgttaaggaagcaaatggttgtaactttgtaagtacttataacatggtgtatctttttgcttatgaatattctgtattataaccattgtttctgtagtttaattaaaacattttcttggtgttagcttttctcagaaaaaaaaaaaaaaaaaaaaaaaaaaaaaaaaaaaaaaaaaa

SEQ ID NO: 16 - protein, 221 aa, Homo sapiens
MSYGRPPPDVEGMTSLKVDNLTYRTSPDTLRRVFEKYGRVGDVYIPRDRYTKESRGFAFVRFHDKRDAEDAMDAMDGAVLDGRELRVQMARYGRPPDSHHSRRGPPPRRYGGGGYGRRSRSPRRRRRSRSRSRSRSRSRSRSRYSRSKSRSRTRSRSRSTSKSRSARRSKSKSSSVSRSRSRSRSRSRSRSPPPVSKRESKSRSRSKSPPKSPEEEGAVSS

SEQ ID NO: 17 - DNA, 2402 bp, Homo sapiens
gggctgaggaggcggggcctgggaggggacaaagccgggaagaggaaaagctcggacctaccctgtggtcccgggtttctgcagagtctacttcagaagcggaggcactgggagtccggtttgggattgccaggctgtggttgtgagtctgagcttgtgagcggctgtggcgccccaactcttcgccagcatatcatcccggcaggcgataaactacattcagttgagtctgcaagactgggaggaactggggtgataagaaatctattcactgtcaaggtttattgaagtcaaaatgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtatgagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaactgttgattttaattaaaattaagtgctaattaccattaaaaaaaaaa

SEQ ID NO: 18 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 19 - DNA, 1578 bp, Homo sapiens
attttgcaacgccataggcttccagcgactgctggtgatgtttctgatgccgacaaaaggatcaaggtggcgaagcccgtggtggagatggatggtgatgagatgacccgtattatctggcagttcatcaaggagaagctcatcctgccccacgtggacatccagctaaagtattttgacctcgggctcccaaaccgtgaccagactgatgaccaggtcaccattgactctgcactggccacccagaagtacagtgtggctgtcaagtgtgccaccatcacccctgatgaggcccgtgtggaagagttcaagctgaagaagatgtggaaaagtcccaatggaactatccggaacatcctgggggggactgtcttccgggagcccatcatctgcaaaaacatcccacgcctagtccctggctggaccaagcccatcaccattggcaggcacgcccatggcgaccagtacaaggccacagactttgtggcagaccgggccggcactttcaaaatggtcttcaccccaaaagatggcagtggtgtcaaggagtgggaagtgtacaacttccccgcaggcggcgtgggcatgggcatgtacaacaccgacgagtccatctcaggttttgcgcacagctgcttccagtatgccatccagaagaaatggccgctgtacatgagcaccaagaacaccatactgaaagcctacgatgggcgtttcaaggacatcttccaggagatctttgacaagcactataagaccgacttcgacaagaataagatctggtatgagcaccggctcattgatgacatggtggctcaggtcctcaagtcttcgggtggctttgtgtgggcctgcaagaactatgacggagatgtgcagtcagacatcctggcccagggctttggctcccttggcctgatgacgtccgtcctggtctgccctgatgggaagacgattgaggctgaggccgctcatgggaccgtcacccgccactatcgggagcaccagaagggccggcccaccagcaccaaccccatcgccagcatctttgcctggacacgtggcctggagcaccgggggaagctggatgggaaccaagacctcatcaggtttgcccagatgctggagaaggtgtgcgtggagacggtggagagtggagccatgaccaaggacctggcgggctgcattcacggcctcagcaatgtgaagctgaacgagcacttcctgaacaccacggacttcctcgacaccatcaagagcaacctggacagagccctgggcaggcagtagggggaggcgccacccatggctgcagtggaggggccagggctgagccggcgggtcctcctgagcgcggcagagggtgagcctcacagcccctctctggaggcctttctaggggatgtttttttataagccagatgtttttaaaagcatatgtgtgtttcccctcatggtgacgtgaggcaggagcagtgcgttttacctcagccagtcagtatgttttgcatactgtaatttatattgcccttggaacacatggtgccatatttagctactaaaaagctcttcacaaaa

SEQ ID NO: 20 - protein, 400 aa, Homo sapiens
MDGDEMTRIIWQFIKEKLILPHVDIQLKYFDLGLPNRDQTDDQVTIDSALATQKYSVAVKCATITPDEARVEEFKLKKMWKSPNGTIRNILGGTVFREPIICKNIPRLVPGWTKPITIGRHAHGDQYKATDFVADRAGTFKMVFTPKDGSGVKEWEVYNFPAGGVGMGMYNTDESISGFAHSCFQYAIQKKWPLYMSTKNTILKAYDGRFKDIFQEIFDKHYKTDFDKNKIWYEHRLIDDMVAQVLKSSGGFVWACKNYDGDVQSDILAQGFGSLGLMTSVLVCPDGKTIEAEAAHGTVTRHYREHQKGRPTSTNPIASIFAWTRGLEHRGKLDGNQDLIRFAQMLEKVCVETVESGAMTKDLAGCIHGLSNVKLNEHFLNTTDFLDTIKSNLDRALGRQ

(19) United States
(12) Patent Application Publication
(10) Pub. No.: US 2018/0318303 A1
(43) Pub. Date: Nov. 8, 2018

(54) METHODS FOR TREATING MYELOPROLIFERATIVE DISORDERS

(71) Applicant: Promedior, Inc., Lexington, MA (US)

(72) Inventors: Elizabeth Trehu, Duxbury, MA (US); Richard M. Jack, Waltham, MA (US)

(21) Appl. No.: 15/566,692

(22) PCT Filed: Apr. 15, 2016
(86) PCT No.: PCT/US16/27773

§ 371 Date: Oct. 13, 2017

Related U.S. Application Data
(60) Provisional application No. 62/218,869, filed on Sep. 15, 2015.
(60) Provisional application No. 62/148,005, filed on Apr. 15, 2015.

(51) Int. Cl. A61K 31/519; A61K 38/20; A61K 38/13; A61K 38/21; A61P 19/08; C07K 14/47

(52) U.S. Cl. CPC A61K 31/519; A61K 38/2013; A61K 38/13; A61K 45/06; A61K 38/212; A61P 19/08; C07K 14/47; A61K 38/202

(57) ABSTRACT

In part, the disclosure relates to methods of treating myeloproliferative disorders by administering one or more Serum Amyloid Protein (SAP) proteins. In certain aspects, the method further comprises monitoring treatment efficacy by measuring change in mutant allele burden. In certain aspects, the disclosure relates to methods of treating myelofibrosis in patient sub-populations who carry myelofibrosis-associated mutations in some of their cells by administering an SAP protein.

### CROSS-REFERENCE TO RELATED APPLICATIONS

This application claims priority to and the benefit of U.S. provisional patent application Ser. No. 62/148,005 filed on Apr. 15, 2015 and 62/218,869 filed on Sep. 15, 2015, the disclosures of which are incorporated herein by reference in their entirety.

### BACKGROUND OF THE INVENTION

Myeloproliferative disease (MPD), also referred to as myeloproliferative neoplasms (MPN), refers to a group of disorders characterized by clonal abnormalities of the blood cells, such as blood cells and precursors of the myeloid lineage. Such disorders may impact myeloid, erythroid, and platelet cells. Myeloproliferative disorders can be challenging to diagnose and treat.

In certain proliferative conditions, such as myelofibrosis (MF), replacement of healthy organ tissue by fibrosis results in inadequate organ function, which contributes to the symptoms of the disorder. Myelofibrosis (including primary myelofibrosis, post-polycythemia vera myelofibrosis and post-essential thrombocythemia myelofibrosis) is a clonal myeloproliferative neoplasm, characterized by progressive bone marrow fibrosis and subsequent ineffective erythropoiesis, dysplastic megakaryocyte hyperplasia, and extramedullary hematopoiesis. The typical clinical presentation includes marked splenomegaly, progressive anemia, and constitutional associated with high morbidity and mortality. Moreover, most patients are not suitable transplant candidates.

Until recently, there was no approved medical therapy for MF and most subjects were managed with various combinations of growth factors, immunomodulatory agents, cytotoxic chemotherapy, and steroids. None of these therapies produced significant responses in the majority of subjects. For this reason, no medication has been approved for MF until recently.

Ruxolitinib is a Janus kinase inhibitor, recently approved in the US and EU for the treatment of subjects with intermediate or high-risk myelofibrosis, including primary myelofibrosis (PMF), post-polycythemia vera myelofibrosis (post-PV MF) and post-essential thrombocythemia (post-ET MF) (JAKAFI® Full Prescribing Information 2011). Treatment with ruxolitinib results in reduction in spleen volume and improvement in constitutional symptoms, but does not appear to have an effect on bone marrow fibrosis or on allele burden.

There is a clear unmet medical need for new therapies that could improve bone marrow fibrosis in subjects with myelofibrosis with a resultant improvement in blood counts and other disease-related factors. While a number of drugs have been developed and evaluated in MF clinical trials, none of the drugs so far have displayed a selective anti-clonal effect, despite activity in alleviating other symptoms. Therefore, a need remains for developing additional therapeutic options for the treatment of myeloproliferative disorders such as myelofibrosis.

### SUMMARY OF THE INVENTION

The disclosure provides various methods, such as various methods of treating a myeloproliferative disorder. The methods include various methods of administering serum amyloid P (SAP) protein or pentraxin-2, such as administering SAP protein to a subject in multiple doses according to a dosage regimen. Optionally, the methods include one or more steps in which one or more genes in a sample taken from the subject are evaluated to determine mutational status (e.g., whether some of the subject's cells comprise a mutation associated with the myeloproliferative disorder). In certain embodiments, subjects having a certain mutational status are specifically selected for treatment or the dosage regimen is adjusted based on mutational status. In other embodiments, mutational status is evaluated over the course of treatment to determine impact of treatment on allele burden. In some embodiments, the dosage regimen is adjusted based on the patient's responsiveness, such as impact on allele burden or impact on one or more other measures of symptom improvement.

In certain aspects, the disclosure provides a method of treating a myeloproliferative disorder, the method comprising administering to a subject in need thereof an effective amount of a serum amyloid P (SAP) protein, wherein some of the subject's cells comprise a mutation associated with the myeloproliferative disorder in one or more genes selected from: JAK2, MPL, CALR, ASXL1, EZH2, SRSF2, IDH1, or IDH2. In other words, some of the subject's cells carry a mutation associated with the myeloproliferative disorder (e.g., the subject comprises cells carrying a mutation in one or more of the foregoing genes). In some embodiments, the subject comprises more than one mutation associated with the myeloproliferative disorder, such as a mutation in two or three (or more than three) of the foregoing genes. In certain embodiments, administering comprises administering the SAP protein in multiple doses according to a dosing schedule and/or dosage regimen, such as to achieve a therapeutic effect.

In certain aspects, the disclosure provides a method of treating a myeloproliferative disorder, comprising administering to a subject in need thereof an effective amount of a serum amyloid P (SAP) protein, wherein some of the subject's cells comprise a mutation associated with the myeloproliferative disorder in one or more genes selected from: JAK2, MPL, CALR, ASXL1, EZH2, SRSF2, IDH1, or IDH2, and wherein the SAP protein is administered according to a dosage regimen effective to reduce mutant allele burden of said gene in said subject. In other words, some of the subject's cells carry a mutation associated with the myeloproliferative disorder (e.g., the subject comprises cells carrying a mutation in one or more of the foregoing genes). In some embodiments, the subject comprises more than one mutation associated with the myeloproliferative disorder, such as a mutation in two or three (or more than three) of the foregoing genes. In certain embodiments, the dosage regimen is also effective to improve one or more other manifestations of the myeloproliferative disorder, such as effective to reduce bone marrow fibrosis by at least one grade.

In certain aspects, the disclosure provides a method for reducing mutant allele burden in a subject having a myeloproliferative disorder, the method comprising administering to a subject in need thereof an effective amount of a serum amyloid P (SAP) protein, wherein the subject comprises a mutation associated with the myeloproliferative disorder in one or more genes selected from: JAK2, MPL, CALR, ASXL1, EZH2, SRSF2, IDH1, or IDH2. In other words, some of the subject's cells carry a mutation associated with the myeloproliferative disorder (e.g., the subject comprises cells carrying a mutation in one or more of the foregoing genes). In some embodiments, the subject comprises more than one mutation associated with the myeloproliferative disorder, such as a mutation in two or three (or more than three) of the foregoing genes. In certain embodiments, the dosage regimen is also effective to improve one or more other manifestations of the myeloproliferative disorder, such as effective to reduce bone marrow fibrosis by at least one grade. In certain embodiments, administering an effective amount comprises administering SAP protein according to a dosage regimen (e.g., multiple doses according to a dosing schedule).

In certain aspects, the disclosure provides a method for treating a myeloproliferative disorder with a serum amyloid P (SAP) protein, the method comprising: (i) measuring a first mutant allele burden of a mutation in one or more genes associated with the myeloproliferative disorder selected from: JAK2, MPL, CALR, ASXL1, EZH2, SRSF2, IDH1, or IDH2, wherein said first mutant allele burden is measured before administration of the SAP protein; (ii) measuring a second mutant allele burden of the same mutation measured in (i), wherein said second mutant allele burden is measured after administration of the SAP protein; and (iii) identifying a difference between the second mutant allele burden and the first mutant allele burden, wherein a decrease in the second mutant allele burden relative to the first mutant allele burden indicates that the administration of the SAP protein is effective in treating the mycloproliferative disorder. In some embodiments, mutant allele burden in more than one gene is measured (e.g., in two, three or more than three genes). In certain embodiments, the measuring performed in step (ii) is performed following approximately 1 cycle of treatment and/or following about 1 month of treatment. In other embodiments, the measuring performed in step (ii) is performed following approximately 2 or 3 cycles of treatment and/or following about 2 or 3 months of treatment. However, step (ii) may be performed sooner or later in the course of treatment. Moreover, the disclosure contemplates that allele burden may be evaluated overtime of treatment to ascertain decrease in allele burden and durability of response.

In certain aspects, the disclosure provides a method for treating a myeloproliferative disorder, comprising: (i) determining whether the cells of a subject having a myeloproliferative disorder comprise a mutation associated with the myeloproliferative disorder in one or more genes selected from: JAK2, MPL, CALR, ASXL1, EZH2, SRSF2, IDH1, or IDH2; and if the subject carries said mutant allele (ii) administering an effective amount of a serum amyloid P (SAP) protein to the subject. In other words, some of the subject's cells carry a mutation associated with the myeloproliferative disorder (e.g., the subject comprises cells carrying a mutation in one or more of the foregoing genes). In some embodiments, the subject comprises more than one mutation associated with the myeloproliferative disorder, such as a mutation in two or three (or more than three) of the foregoing genes. In some embodiments, the mutation associated with the myeloproliferative disorder is not JAK2V617F. In certain embodiments, administering comprises administering the SAP protein in multiple doses according to a dosing schedule and/or dosage regimen, such as to achieve a therapeutic effect. In certain embodiments, the dosage regimen is effective to decrease allele burden and/or to improve one or more other manifestations of the myeloproliferative disorder, such as effective to reduce bone marrow fibrosis by at least one grade.

In some embodiments of any of the foregoing or following, the mutation associated with the myeloproliferative disorder is an activating mutation. In some embodiments, the subject comprises more than one mutation associated with a myeloproliferative disorder, such as a mutation in more than one of the foregoing genes (e.g., two, three, more than three). In some embodiments, the subject is a subject in need of treatment for a myeloproliferative disorder (e.g., a subject in need thereof).

In some embodiments of any of the foregoing or following, the subject comprises a mutation at codon 617 of JAK2 (e.g., the mutation associated with the myeloproliferative disorder is JAK2V617F). In some embodiments, the subject comprises a mutation in exon 12 or exon 14 of JAK2. In some embodiments, the subject comprises a mutation at codon 515 of MPL. In some embodiments, the mutation results in a W515L, W515K, W515A, or W515R amino acid substitution in MPL. In some embodiments, the subject comprises a mutation in exon 10 of MPL. In some embodiments, the subject comprises a mutation a mutation in exon 9 of CALR. In some embodiments, the subject comprises a mutation in exon 12 of ASXL1. In some embodiments, the subject comprises a mutation in exon 12 of ASXL1. In some embodiments, the subject comprises a mutation in exon 4 of IDH1. In some embodiments, the subject comprises a mutation at codon 132 of IDH1. In some embodiments, the subject comprises a mutation in exon 4 of IDH2. In some embodiments, the subject comprises a mutation at codon 140 of IDH2. In some embodiments, the subject comprises a mutation at codon 172 of IDH2. In some embodiments, the mutation is not JAK2V617F. In some embodiments, if the mutation is JAK2V61F, the subject also comprises one or more additional mutations associated with the myeloproliferative disorder. In certain embodiments, the subject comprises more than one mutation associated with a myeloproliferative disorder, such as a mutation in more than one of the foregoing genes (e.g., two, three, more than three). In certain embodiments, the subject comprises or is evaluated for mutations in JAK2, CALR and MPL and, optionally, one or more other genes. It should be understood that referring to “the subject comprises” also refers to the subject comprising cells that comprise the mutation, or the subject comprising cells that carry the mutation.

In some embodiments of any of the foregoing or following, the mutation associated with the myeloproliferative disorder is a deletion, insertion, point mutation, or translocation. In some embodiments, the mutation results in the absence of expression of the one or more proteins encoded by the one or more genes or in the expression of a truncated protein. In some embodiments, the mutation is an activating mutation. In some embodiments, the activating mutation is JAK2V617F. In some embodiments, the activating mutation is a mutation at codon 515 of MPL.

In some embodiments of any of the foregoing or following, the mutation associated with the myeloproliferative disorder is present on one or both alleles of the one or more genes.

In some embodiment of any of the foregoing or following, SAP protein is administering in multiple doses, such as according to a dosing schedule and/or dosage regimen. Exemplary dosage regimens are provided herein.

In some embodiments of any of the foregoing or following, prior to and/or following administration of an SAP protein, one or more manifestations of the myeloproliferative disorder are measured in the subject, such as allele burden, bone marrow fibrosis and the like. Exemplary other manifestations/end points are provided herein. In some embodiments, administration of the SAP protein is effective to improve one or more of these manifestations/end points, optionally, without adverse myelosuppression.

In some embodiments of any of the foregoing or following, the decrease in mutant allele burden following treatment with SAP protein in one or more of the myeloproliferative disorder-associated genes is 10 to 90%. In some embodiments, the decrease in mutant allele burden is 25% to 50%. In some embodiments, the decrease in mutant allele burden is at least 50%, such as about 50-60%, 50-70%, 50-75%, or 50-80%. In some embodiments, a complete molecular response is observed. In certain embodiments, the decrease in mutant allele burden is evaluated after one treatment cycle or after 30 days. In certain embodiments, the decrease in mutant allele burden is evaluated 60 days, 90 days or 120 days after initiation of treatment. In some embodiments, decrease in mutant allele burden is evaluated at multiple points. In some embodiments, in addition to mutant allele burden, one or more other symptoms are evaluated before and/or during treatment.

In some embodiments of any of the foregoing or following, the biological sample is a blood sample. In some embodiments, the biological sample is bone marrow. In other words, in certain embodiments, when a sample is taken from a subject for, for example, the purpose of evaluating the presence of a mutation or for evaluating allele burden, the sample is a blood sample or a bone marrow sample. In certain embodiments, a blood sample or bone marrow sample is taken from a subject to evaluate other manifestations of the myeloproliferative disorder, such as bone marrow fibrosis.

In some embodiments of any of the foregoing or following, the measuring step comprises amplifying nucleic acid comprising all or a portion of the one or more genes associated with the myeloproliferative disorder. In other words, in certain embodiments, when a gene associated with myeloproliferative disorder is being assayed, for example, to determine whether a subject carries a mutation associated with the myeloproliferative disorder or to evaluate allele burden or changes in allele burden, the evaluation may comprise amplifying nucleic acid comprising all or a portion of the one or more genes associated with the myeloproliferative disorder. In certain embodiments, multiple candidate genes are evaluated or measured as part of the assay or method (e.g., two, three or more than three).

In some embodiments of any of the foregoing or following, the measuring step comprises determining the proportion of mutant nucleic acid to wildtype nucleic acid of the one or more genes associated with the myeloproliferative disorder.

In certain embodiments of any of the foregoing or following, the SAP protein is an SAP protein comprising one or more protomers. In certain embodiments, the SAP protein is an SAP protein comprising five protomers. In certain embodiments, the SAP protein is provided as a composition comprising SAP protein, such as a pharmaceutical composition, and the SAP proteins and compositions may be used in any of the methods described herein.

In some embodiments of any of the foregoing or following, SAP protein is a glycosylated human SAP protein. By way of example, the SAP protein may comprise an SAP polypeptide, such as a glycosylated human SAP polypeptide, such as a glycosylated human SAP polypeptide having glycosylation that differs from SAP protein isolated from human serum (e.g., human SAP comprising an N-linked oligosaccharide chain, wherein at least one branch of the oligosaccharide chain terminates with a α2,3-linked sialic acid moiety). In certain embodiments, the SAP protein is recombinant human SAP (e.g., rhSAP).

In certain embodiments, the SAP protein comprises the recombinant human SAP also known in the art as PRM-151. Duffield and Lupher, Drug News & Perspectives 2010, 23(5):305-315. Optionally, rhSAP may be prepared in CHO cells or in another suitable cell line. Any of the methods described herein comprise, in certain embodiments, administering the recombinant human SAP known as PRM-151.

In some embodiments of any of the foregoing or following, the SAP protein is a glycosylated human SAP protein comprising an N-linked oligosaccharide chain, wherein at least one branch of the oligosaccharide chain terminates with a α2,3-linked sialic acid moiety. In some embodiments, all the sialylated branches of the oligosaccharide chain terminate with α2,3-linked sialic acid moieties. In some embodiments, the oligosaccharide chain is substantially free of α2,6-linked sialic acid moieties. By way of example, the SAP protein may comprise such a glycosylated human SAP protein. In some embodiments, the glycosylated human SAP comprises recombinant human SAP also referred to as recombinant human pentraxin-2 (hPTX-2), as described in Duffield and Lupher, Drug News & Perspectives 2010, 23(5):305-315. In certain embodiments, the methods of the disclosure comprise administering a composition comprising glycosylated human SAP protein, wherein the human SAP protein comprises five SAP protomers. In certain embodiments, the composition SAP protein comprising an N-linked oligosaccharide chain, wherein at least one branch of the oligosaccharide chain terminates with a α2,3-linked sialic acid moiety. In certain embodiments, the SAP protein comprises five SAP protomers, wherein each protomer comprises an N-linked oligosaccharide chain, wherein at least one branch of the oligosaccharide chain terminates with a α2,3-linked sialic acid moiety. In certain embodiments, the composition comprising the SAP protein or the SAP protein comprises 85% less α2,6-linked sialic acid in comparison to serum-derivated SAP. In some embodiments, all the sialylated branches of all of the oligosaccharide chains terminate with α2,3-linked sialic acid moieties. In some embodiments, the oligosaccharide chains are substantially free of α2,6-linked sialic acid moieties.

In some embodiments of any of the foregoing or following, the SAP protein comprises an amino acid sequence at least 85% identical to SEQ ID NO: 1. In some embodiments, the SAP protein comprises an amino acid sequence at least 95% identical to SEQ ID NO: 1. In some embodiments, the SAP protein is a glycosylated SAP protein having glycosylation that differs from human SAP purified from serum. In some embodiments, the SAP protein comprises five polypeptide chains each of which comprise an amino acid sequence at least 85%, at least 90%, 95%, 98%, or even 100% identical to SEQ ID NO: 1. In certain embodiments, the SAP protein comprises five SAP protomers and each protomer comprises an amino acid sequence that is at least 85% (or is at least 90, 95, 98, 99 or 100%) identical to SEQ ID NO: 1.

In some embodiments of any of the foregoing or following, the SAP protein is a fusion protein comprising an SAP domain and one or more heterologous domains. In some embodiments, the one or more heterologous domains enhance one or more of in vivo stability, in vivo half-life, uptake/administration, tissue localization or distribution, formation of protein complexes, and/or purification.

In some embodiments of any of the foregoing or following, the SAP protein comprises one or more modified amino acid residues. In some embodiments, the one or more modified amino acid residues comprise a PEGylated amino acid, a prenylated amino acid, an acetylated amino acid, a biotinylated amino acid, and/or an amino acid conjugated to an organic derivatizing agent.

In some embodiments of any of the foregoing or following, the SAP protein is administered by a mode selected from: orally, topically, by injection, by intravenous injection, by subcutaneous injection, by inhalation, continuous release by depot or pump, or a combination thereof.

In some embodiments of any of the foregoing or following, the method further comprises administering to the patient an anti-cancer therapeutic (e.g., an additional anti-cancer therapeutic).

In some embodiments of any of the foregoing or following, the anti-cancer therapeutic is selected from: chemotherapy agents, antibody-based agents, kinase inhibitors (e.g., tyrosine kinase inhibitors, serine/threonine kinase inhibitors, etc.), immunomodulatory agents, biologic agents, and combinations thereof. A single additional agent or multiple additional agents or treatment modalities may be co-administered (at the same or differing time points and/or via the same or differing routes of administration and/or on the same or a differing dosing schedule). In certain embodiments, the combination of an SAP protein and the additional anti-cancer therapeutic is indicated for a condition, patient population or sub-population for which the additional anti-cancer therapeutic alone is not indicated. In certain embodiments, the SAP protein comprises a glycosylated SAP protein, such as an SAP protein having glycosylation that differs from human SAP purified from serum.

In some embodiments of any of the foregoing or following, the chemotherapy agent is selected from but not limited to: actinomycin D, aldesleukin, alitretinoin, all-trans retinoic acid/ATRA, altretamine, amascrine, asparaginase, azacitidine, azathioprine, bacillus calmette-guerin/BCG, bendamustine hydrochloride, bexarotene, bicalutamide, bleomycin, bortezomib, busulfan, capecitabine, carboplatin, carfilzomib, carmustine, chlorambucil, cisplatin/cisplatinum, cladribine, cyclophosphamide/cytophosphane, cytabarine, dacarbazine, daunorubicin/daunomycin, denileukin diftitox, dexrazoxane, docetaxel, doxorubicin, epirubicin, etoposide, fludarabine, fluorouracil (5-FU), gemcitabine, goserelin, hydrocortisone, hydroxyurea, idarubicin, ifosfamide, interferon alfa, irinotecan CPT-11, lapatinib, lenalidomide, leuprolide, mechlorethamine/chlormethine/mustine/HN2, mercaptopurine, methotrexate, methylprednisolone, mitomycin, mitotane, mitoxantrone, octreotide, oprelvekin, oxaliplatin, paclitaxel, pamidronate, pegaspargase, pegfilgrastim, PEG interferon, pemetrexed, pentostatin, phenylalanine mustard, plicamycin/mithramycin, prednisone, prednisolone, procarbazine, raloxifene, romiplostim, sargramostim, streptozocin, tamoxifen, temozolomide, temsirolimus, teniposide, thalidomide, thioguanine, thiophosphoamide/thiotepa, thiotepa, topotecan hydrochloride, toremifene, tretinoin, valrubicin, vinblastine, vincristine, vindesine, vinorelbine, vorinostat, zoledronic acid, and combinations thereof. In certain embodiments, the method comprises administration of the SAP protein and an additional anti-cancer therapeutic, which additional anti-cancer therapeutic is a chemotherapeutic agent, such as a single chemotherapeutic agent or a combination of two or more chemotherapeutic agents. In certain embodiments, the SAP protein comprises a glycosylated SAP protein, such as an SAP protein having glycosylation that differs from human SAP purified from serum. In certain embodiments, the chemotherapeutic agent is selected from the group consisting of any of the foregoing agents.

In some embodiments of any of the foregoing or following, the antibody-based agent is selected from but not limited to: alemtuzumab, bevacizumab, cetuximab, fresolimumab, gemtuzumab ozogamicin, ibritumomab tiuxetan, ipilimumab, ofatumumab, panitumumab, rituximab, tositumomab, trastuzumab, trastuzumab DM1, and combinations thereof. In certain embodiments, the method comprises administration of the SAP protein and an additional anti-cancer therapeutic, which additional anti-cancer therapeutic is an antibody-based agent. In certain embodiments, the SAP protein comprises a glycosylated SAP protein, such as an SAP protein having glycosylation that differs from human SAP purified from serum. In certain embodiments, the chemotherapeutic agent is selected from the group consisting of any of the foregoing agents.

In some embodiments of any of the foregoing or following, the kinase inhibitor (e.g., tyrosine kinase inhibitors, serine/threonine kinase inhibitors, etc.) is selected from but not limited to: axitinib, bafetinib, bosutinib, cediranib, crizotinib, dasatinib, erlotinib, gefitinib, imatinib, lapatinib, neratinib, nilotinib, pazopanib, ponatinib, quizartinib, regorafenib, sorafenib, sunitinib, vandetanib, vatalanib, vemurafinib, and combinations thereof. In certain embodiments, the method comprises administration of the SAP protein and an additional anti-cancer therapeutic, which additional anti-cancer therapeutic is a kinase inhibitor. In certain embodiments, the SAP protein comprises a glycosylated SAP protein, such as an SAP protein having glycosylation that differs from human SAP purified from serum. In certain embodiments, the chemotherapeutic agent is selected from the group consisting of any of the foregoing agents.

In some embodiments of any of the foregoing or following, the immunomodulatory agent is selected from but not limited to: thalidomide, lenalidomide, pomalidomide, methotrexate, leflunomide, cyclophosphamide, cyclosporine A, minocycline, azathioprine, tacrolimus, methylprednisolone, mycophenolate mofetil, rapamycin, mizoribine, deoxyspergualin, brequinar, 5,6-dimethylxanthenone-4-acetic acid (DMXAA), lactoferrin, poly AU, polyI:polyC12U, poly-ICLC, imiquimod, resiquimod, unmethylated CpG dinucleotide (CpG-ODN), and ipilumumab. In certain embodiments, the method comprises administration of the SAP protein and an additional anti-cancer therapeutic, which additional anti-cancer therapeutic is an immunomodulatory agent. In certain embodiments, the SAP protein comprises a glycosylated SAP protein, such as an SAP protein having glycosylation that differs from human SAP purified from serum. In certain embodiments, the chemotherapeutic agent is selected from the group consisting of any of the foregoing agents.

In some embodiments of any of the foregoing or following, the kinase inhibitor is a Janus kinase inhibitor selected from but not limited to: AC-430, AZD1480, baricitinib. BMS-911453. CEP-33779, CYT387, GLPG-0634, INCB18424, lestaurtinib. LY2784544, NS-018, pacritinib, ruxolitinib, TG101348 (SAR302503), tofacitinib, VX-509, R-348, R723 and combinations thereof. In certain embodiments, the method comprises administration of the SAP protein and an additional anti-cancer therapeutic, which additional anti-cancer therapeutic is a Janus kinase inhibitor. In certain embodiments, the SAP protein comprises a glycosylated SAP protein, such as an SAP protein having glycosylation that differs from human SAP purified from serum. In certain embodiments, the chemotherapeutic agent is selected from the group consisting of any of the foregoing agents.

In some embodiments of any of the foregoing or following, the biologic agent is selected from but not limited to: IL-2, IL-3, erythropoietin, G-CSF, filgrastim, interferon alfa, bortezomib and combinations thereof. In certain embodiments, the chemotherapeutic agent is selected from the group consisting of any of the foregoing agents.

In some embodiments of any of the foregoing or following, the anti-cancer therapeutic is selected from but not limited to: AB0024, AZD1480, AT-9283, BMS-911543, CYT387, everolimus, givinostat, imetelstat, lestaurtinib, LY2784544, NS-018, oral arsenic, pacritinib, panobinostat, peginterferon alfa-2a, pomalidomide, pracinostat, ruxolitinib, TAK-901, and TG101438 (SAR302503). In certain embodiments, the chemotherapeutic agent is selected from the group consisting of any of the foregoing agents.

In some embodiments, the SAP protein and the one or more additional active agents (e.g., the additional anti-cancer therapeutic) are co-formulated. In some embodiments, the SAP protein and the one or more additional active agents are administered simultaneously. In some embodiments, the SAP protein and the one or more additional active agents are administered within a time of each other to produce overlapping therapeutic effects in the patient. When the SAP protein and the one or more additional active agents are administered simultaneously or within a time of each other to produce overlapping therapeutic effects, the agents may be administered by the same or a different route of administration (e.g., oral versus infusion).

In some embodiments, the myeloproliferative disorder is myelofibrosis. In some embodiments, the myelofibrosis is primary myelofibrosis, post-polycythemia vera myelofibrosis, or post-essential thrombocythemia myelofibrosis. In some embodiments, the myeloproliferative disorder is post-polycythemia vera or post-essential thrombocythemia. In certain embodiments, the methods of the present disclosure involve use of SAP as a monotherapy.

In certain embodiments, prior to initiation of treatment with SAP, the patient has clinically significant anemia and/or thrombocytopenia.

In some embodiments, the myeloproliferative disorder is polycythemia vera, essential thrombocytosis, or chronic myelogenous leukemia.

In certain embodiments, treatment comprises administering the SAP protein according to a dosing schedule, such as any of the dosing schedules described herein. In certain embodiments, administration and/or the therapeutically effective amount is understood in the art to comprise administration according to a dose and dosing schedule effective to produce therapeutic benefit as defined in a clinical study protocol, full prescribing information, the Investigator's Brochure, or by improvement in measures generally understood by experts in the field to be of benefit to patients with the respective disease. In certain embodiments, the SAP protein, whether administered alone or as part of a combination therapy, can be administered according to a dosing schedule providing administration less than once per week. In certain embodiments, such less frequent dosing occurs following an initial loading phase wherein, for example, during the first week of a treatment cycle, the SAP protein is administered multiple times.

In certain embodiments of any of the foregoing, treatment improves organ function (e.g., therapeutic efficacy comprises improvement in organ function. SAP protein is administered alone or in combination and improves organ function). In certain embodiments, the organ is the bone marrow and improvement in organ function is evaluated by assessing improvement in hemoglobin and/or platelets (e.g., improvement in one or both of these metrics evinces improvement in organ function; in the case of platelets, improvement in platelets refers to increasing platelets in subjects suffering from low platelet levels; in the case of hemoglobin, improvement in hemoglobin refers to increasing hemoglobin in subjects suffering from low hemoglobin levels). In certain embodiments, treatment restores normal tissue, such as by decreasing fibrosis (e.g., therapeutic efficacy comprises restoration of normal tissue). In certain embodiments, restoring normal tissue is evaluated by assessing bone marrow fibrosis. In certain embodiments, treatment reduces mutant allele burden.

In certain aspects, the method comprises administering an SAP protein and an additional anti-cancer therapeutic according to a dosage regimen such that one or more side effects are reduced relative to treatment with the additional anti-cancer therapeutic alone.

In certain aspects, the disclosure provides a kit comprising: a) a composition or pharmaceutical composition comprising an SAP protein; b) one or more oligonucleotides capable of amplifying a region of one or more genes selected from: JAK2, MPL, CALR, ASXL1, EZH2, SRSF2, IDH1, and IDH2; and c) instructions for use. In some embodiments, the mutation detected is not JAK2V617F.

The disclosure contemplates all suitable combinations of any of the features of the invention, such as combinations of any of the aspects and embodiments described herein. For example, the disclosure contemplates that any of the foregoing aspects and embodiments may be combined with each other and/or with any of the embodiments disclosed herein. For example, SAP proteins described using any combination of functional and/or structural features may be used alone or in a combination therapy in any of the methods described herein, to treat any of the conditions, patient populations, or sub-populations of patients described herein, such as patients described based on any one or more symptoms.

### DETAILED DESCRIPTION OF THE INVENTION

### Overview

The present disclosure provides methods and kits for evaluating allele burden in a subject having a myeloproliferative disorder, including methods and kits for reducing allele burden and/or using allele burden to evaluate treatment efficacy. The disclosure also provides methods for treating subjects having a myeloproliferative disorder, wherein the subject carriers a mutation in one or more genes (e.g., the subject comprises cells comprising one or more mutations associated with a myeloproliferative disorder).

A number of prognostically-relevant somatic mutations and cytogenetic abnormalities have been found to be associated with myeloproliferative disorders. Prognostically-relevant mutations have been identified in a diverse set of genes including JAK2, MPL, CALR, ASXL1, SRSF2, EZH2, IDH1, and IDH2. Presence of JAK2, MPL, CALR, ASXL1, and SRSF2 mutations were found to be independent predictors of shortened survival in primary myelofibrosis in a recent study (Tefferi et al. ASH 2014 Abstract 406) and triple-negativity for JAK2, MPL and CALR (TN), JAK2 or MPL mutation, and mutations in ASXL1, SRSF2, EZH2 or IDH1/2 were identified as risk factors for inferior survival in another cohort (Vanucchi et al. ASH 2015 Abstract 405). Thus, mutational status is a predictor of poor outcomes.

Cytogenetic abnormalities in primary myelofibrosis were recently stratified into four risk designations: very high risk (MK, inv(3), i(17q), −7/7q-, 11q or 12p abnormalities), high (complex without MK, two abnormalities not included in very high risk category, 5q-, +8, other autosomal trisomies except +9, and other sole abnormalities not included in other risk categories), intermediate (sole abnormalities of 20q-, 1q duplication or any other translocation, and −Y or other sex chromosome abnormality) and low (normal or sole abnormalities of 13q- or +9) (Tefferi et al. ASH 2014 Abstract 631).

Allele burden is the ratio between mutant and wild-type nucleic acid in, for example, hematopoietic cells. So far, JAK inhibitor therapy in humans has had little effect on JAK2V617F allele burden or bone marrow fibrosis (Tefferi. Blood 119(12): 2721-2730). In addition to JAK2V617F, PMF and the other BCR-ABL1-negative myeloproliferative neoplasms are characterized by many other somatic mutations as described above, including MPL, TET2, ASXL1, CBL, IDH1, IDH2, IKZF1, LNK, EZH2, DNMT3A, CUX1, and SF3B1 mutations (Tefferi et al. Mayo Clin Proc. 2012, 87(1):25-33). Quantitative analyses of other prognostically-relevant somatic mutations and cytogenetic analyses will also be useful for monitoring and managing myeloproliferative disorders.

The present disclosure provides new genetics-based therapeutic regimens for treating myeloproliferative disorders using an SAP protein.

Myelofibrosis is characterized by the presence of dense fibrotic tissue in the bone marrow. One goal of therapeutic intervention is to restore normal organ function by preventing or reducing excess fibrotic tissue. The regulation of events leading to fibrosis involves at least two major events. One is the proliferation and differentiation of fibrocytes. Fibrocytes are a distinct population of fibroblast-like cells derived from peripheral blood monocytes that normally enter sites of tissue injury to promote angiogenesis and wound healing. Fibrocytes are important in the formation of tumors, particularly stromal tissue in tumors. Fibrocytes differentiate from CD14+ peripheral blood monocytes, and may differentiate from other PBMC cells. The presence of SAP, IL-12, Laminin-1, anti-FcγR antibodies, crosslinked IgG and/or aggregated IgG may inhibit or at least partially delay this process.

The second major event is the formation and maintenance of fibrotic tissue. Fibrotic tissue may be formed and maintained by the differentiation of monocytes into fibrocytes, macrophages or myofibroblasts, the recruitment and proliferation of fibroblast cells, the formation of new extracellular matrix, and the growth of new vascular tissue. In pathologic fibrosis, such as following chronic inflammation, injury, malignancy, or idiopathic fibrosis, it is this excess fibrotic tissue that can lead to tissue damage and destruction.

Recently, it has been suggested that serum amyloid P (SAP) or pentraxin-2 (PTX-2) can be used as a therapeutic agent to treat various disorders, including fibrosis-related disorders, hypersensitivity disorders, autoimmune disorders, mucositis, and inflammatory disorders such as those caused by microbial infection. See, for example, U.S. Pat. Nos. 8,247,370 and 8,497,243 and U.S. patent application Ser. Nos. 12/720,845 and 12/720,847. SAP binding to FcγR provides an inhibitory signal for fibrocyte, fibrocyte precursor, myofibroblast precursor, and/or hematopoietic monocyte precursor differentiation. The use of SAP and SAP agonists as a therapeutic treatment for fibrosis is described in U.S. Pat. Nos. 7,763,256, and 8,247,370, which are hereby incorporated by reference. In certain embodiments of any of the methods described herein, the method comprises administration of SAP protein (see the Examples). In certain embodiments, the SAP is recombinant human SAP, also referred to as recombinant human pentraxin-2, such as recombinant human SAP produced in CHO cells. In certain embodiments, the SAP protein comprises a human SAP protein, such as a human SAP protein having glycosylation that differs from that of SAP purified from human serum. In certain embodiments, the SAP protein comprises a human SAP protein wherein all the sialylated branches of the N-linked oligosaccharide chains terminate in α2,3-linked sialic acid moieties and/or wherein the N-linked oligosaccharide chains are substantially free of α2,6-linked sialic acid moieties. In certain embodiments, the methods of the disclosure comprise administering a composition comprising glycosylated human SAP protein, wherein the human SAP protein comprises five SAP protomers. In certain embodiments, at least one of the protomers or 1, 2, 3, 4 or 5 such protomers comprise an N-linked oligosaccharide chain, wherein at least one branch of the oligosaccharide chain terminates with a α2,3-linked sialic acid moiety. In certain embodiments, the SAP protein comprises five SAP protomers, wherein each protomer comprises an N-linked oligosaccharide chain, wherein at least one branch of the oligosaccharide chain terminates with a α2,3-linked sialic acid moiety or wherein all of the sialyated branches of the SAP protein terminatein an α2,3 linked sialic acid moiety. In certain embodiments, the composition comprising the SAP protein or the SAP protein comprises 85% less α2,6-linked sialic acid in comparison to serum-derivated SAP. In some embodiments, all the sialylated branches of the oligosaccharide chain(s) terminate with α2,3-linked sialic acid moieties. In some embodiments, the oligosaccharide chain(s) is substantially free of α2,6-linked sialic acid moieties.

The present disclosure provides methods for treating myeloproliferative disorders. The method generally involves administering an effective amount of an anti-fibrotic agent such as an SAP protein, as a single agent, or in combination with an additional agent.

In some embodiments, an effective amount of an SAP protein is an amount that, when administered alone, or in combination therapy, is effective to reduce mutant allele burden by at least about 10%, and more preferably at least about 15%, 20%, 25%, 30%, 35%, 40%, 45%, or even at least about 50%, 60%, 70%, 80%, 90% or more, compared with the mutant allele burden in the individual prior to treatment with the SAP protein. In certain embodiments, the reduction in mutant allele burden is about 10%-90%, 10%-75%, 10%-50%, 20%-75%, 30%-75%, 20%-50%, 30%-60%, and the like. In certain embodiments, the SAP protein is administered in multiple dosing according to a dosing schedule and/or dosage regimen, and the reduction in allele burden is evaluated after multiple doses (e.g., after about 1, 2, 3, 4 or 5 months of treatment). In certain embodiments, the SAP protein is administered according to a dosing schedule and/or dosage regimen, and when administered alone or in a combination therapy, is effective to reduce fibrosis by at least about 10%, and more preferably at least about 15%, 20%, 25%, 30%, 35%, 40%, 45%, or even at least about 50%, or more, compared with the degree of fibrosis in the individual prior to treatment with SAP. In certain embodiment fibrosis is bone marrow fibrosis, the effective treatment is a reduction in bone marrow fibrosis by at least one grade, optionally, at least 2 grades. In certain embodiments, the SAP protein is administered in multiple doses according to a dosing schedule and/or dosage regimen, and the reduction in fibrosis is evaluated after multiple doses (e.g., after about 1, 2, 3, 4 or 5 months of treatment).

Methods of the disclosure are useful in evaluating the interaction between selected genetic mutations and cytogenetic abnormalities prevalent in myeloproliferative disorders and an SAP protein. Methods of the disclosure are useful in evaluating the association of baseline mutational status or cytogenetic abnormalities and response to treatment with an SAP protein. In some embodiments, the SAP protein comprises a glycosylated SAP protein (e.g., SAP comprising a glycosylated SAP protein, such as a glycosylated SAP protein having glycosylation that differs from that of SAP purified from human serum; recombinant human SAP, such as recombinant human pentraxin-2 or PRM-151).

### Definitions

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and nucleic acid chemistry and hybridization are those well known and commonly employed in the art. Standard techniques are used for nucleic acid and peptide synthesis. The techniques and procedures are generally performed according to conventional methods in the art and various general references (e.g., Sambrook et al., 1989, Molecular Cloning: A Laboratory Manual, 2d ed. Cold Spring Harbor Laboratory Press, Cold Spring Harbor, N.Y.), which are provided throughout this document.

The articles “a” and “an” are used herein to refer to one or to more than one (i.e., to at least one) of the grammatical object of the article. By way of example, “an element” means one element or more than one element.

As used herein, the term “about” means plus or minus 10% of the numerical value of the number with which it is being used. Therefore, about 50% means in the range of 45%-55%.

As used herein, the term “substantially” means being largely but not wholly what is specified. For example, the term “substantially similar” with regard to a nucleotide sequence indicates that the sequence is largely identical to another reported sequence for the same protein or peptide; however, the nucleotide sequence may include any number of variations or mutations that do not affect the structure or function of the resulting protein.

“Administering,” when used in conjunction with a therapeutic, means to administer a therapeutic directly into or onto a target tissue or to administer a therapeutic to a patient, whereby the therapeutic can impact the patient. Thus, as used herein, the term “administering,” when used in conjunction with an SAP protein can include, but is not limited to, providing an SAP protein to a subject systemically by, for example, intravenous injection (e.g., which may be intravenous infusion), subcutaneous delivery (e.g., subcutaneous injection or implantation of a subcutaneous delivery device), whereby the therapeutic reaches the target tissue.

“Administering” a composition may be accomplished by, for example, intravenous, subcutaneous, intramuscular, or intralesional injection, oral administration, topical administration, or by these methods in combination with other known techniques. Such combination techniques include heating, radiation, ultrasound and the use of delivery agents. When more than one different therapeutic agent is administered, the agents may be administered by the same or different routes of administration and/or at the same or differing times. As is understood in the art, an agent can be administered according to a dosing schedule.

As used herein, the term “dosage regimen” encompasses both the dose or dosage (i.e., the amount of the SAP protein) and the dosing schedule (i.e., the frequency of administration or intervals between successive doses of the SAP protein).

“Providing,” when used in conjunction with a therapeutic, means to administer a therapeutic directly into or onto a target tissue, or to administer a therapeutic to a patient whereby the therapeutic can impact the patient.

The term “improves” is used to convey that the present disclosure changes either the characteristics and/or the physical attributes of the tissue to which it is being provided, applied or administered. The term “improves” may also be used in conjunction with a diseased state such that when a diseased state is “improved” the symptoms, manifestations, or physical characteristics associated with the diseased state are diminished, reduced or eliminated.

As used herein, “isolated” means altered or removed from the natural state through human intervention. For example, SAP naturally present in a living animal is not “isolated,” but a synthetic SAP protein or recombinant SAP protein, or an SAP protein partially or completely separated from the coexisting materials of its natural state is “isolated.” An isolated SAP protein can exist in substantially purified form, or can exist in a non-native environment such as, for example, a cell into which the SAP protein has been delivered.

The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics, as further described below.

As used herein, the term “nucleic acid” refers to a polynucleotide such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotide.

“Optional” or “optionally” means that the subsequently described event or circumstance may or may not occur, and that the description includes instances where the event occurs and instances where it does not.

The terms “peptides”, “proteins” and “polypeptides” are used interchangeably herein. The term “purified protein” refers to a preparation of a protein or proteins that are preferably isolated from, or otherwise substantially free of, other proteins normally associated with the protein(s) in a cell or cell lysate. The term “substantially free of other cellular proteins” or “substantially free of other contaminating proteins” is defined as encompassing individual preparations of each of the proteins comprising less than 20% (by dry weight) contaminating protein, and preferably comprises less than 5% contaminating protein. Functional forms of each of the proteins can be prepared as purified preparations by using a cloned gene as is well known in the art. By “purified”, it is meant that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

By “pharmaceutically acceptable,” “physiologically tolerable,” and grammatical variations thereof, as they refer to compositions, carriers, diluents, and reagents or other ingredients of the formulation, can be used interchangeably and indicate that the materials are capable of administration without the production of undesirable physiological effects such as nausea, dizziness, rash, gastric upset or other deleterious effects to the recipient thereof.

“Pharmaceutically acceptable salts” include both acid and base addition salts. “Pharmaceutically acceptable acid addition salt” refers to those salts which retain the biological effectiveness and properties of the free bases and which are not biologically or otherwise undesirable and formed with inorganic acids, such as hydrochloric acid, hydrobromic acid, sulfuric acid, nitric acid, carbonic acid, phosphoric acid, and the like. Organic acids may be selected from aliphatic, cycloaliphatic, aromatic, araliphatic, heterocyclic, carboxylic, and sulfonic classes of organic acids, such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid, and the like.

As used herein, the term “pharmaceutically acceptable salts, esters, amides, and prodrugs” refers to those carboxylate salts, amino acid addition salts, esters, amides, and prodrugs of the compounds which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the disclosure.

As used herein, the term “therapeutic” means an agent utilized to treat, combat, ameliorate, prevent or improve an unwanted condition or disease of a patient. In part, embodiments of the present disclosure are directed to the treatment of myeloproliferative diseases, or the aberrant proliferation of cells.

An “effective amount” of a composition is a predetermined amount calculated to achieve the desired result. An effective amount is an amount that is consistent with a dosage regimen that, over a period of time, yields a desired therapeutic effect. For an effective amount to be therapeutically effective, multiple doses over time may be required. In certain embodiments herein, when an effective amount is specific a therapeutically effective amount may be referred to as well. A desired result may be the maintenance, amelioration or resolution of symptoms, manifestations, or any of the effects described herein, or any of the effects commonly recognized in the art as a useful effect. The activity contemplated by the present methods includes both medical therapeutic and/or prophylactic treatment, as appropriate. The specific dose of a compound administered according to this disclosure to obtain therapeutic and/or prophylactic effects will, of course, be determined by the particular circumstances surrounding the case, including, for example, the compound administered, the route of administration, and the condition being treated. An effective amount of compound of this disclosure is typically an amount such that when it is administered in a physiologically tolerable excipient composition, it is sufficient. “Therapeutically effective amounts” may be administered according to a dosing schedule. It is understood that when administering a drug according to a dosing schedule, it may take some period of time before improvement in symptoms or manifestations is observed. Nevertheless, administration in one or more doses that, alone or in combination, results in or is intended to result in improvement in symptoms or manifestations and/or decrease in allele burden are exemplary of administering an effective amount.

“N-linked” oligosaccharides are those oligosaccharides that are linked to a peptide backbone through asparagine, by way of an asparagine-N-acetylglucosamine linkage. N-linked oligosaccharides are also called “N-glycans.” Naturally occurring N-linked oligosaccharides have a common pentasaccharide core of Man[(α1,6-)-(Man(α1,3)]-Man(β1,4)-GlcNAc(β1,4)-GlcNAc(β1,N). They differ in the presence of, and in the number of branches (also called antennae) of peripheral sugars such as N-acetylglucosamine, galactose, N-acetylgalactosamine, fucose, and sialic acid. Optionally, this structure may also contain a core fucose molecule and/or a xylose molecule.

The term “sialic acid” refers to any member of a family of nine-carbon carboxylated sugars. The most common member of the sialic acid family is N-acetyl-neuraminic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁C₆-acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For a review of the sialic acid family, see, e.g., Varki, Glycobiology 2: 25-40 (1992); Sialic Acids: Chemistry, Metabolism and Function, R Schauer, Ed. (Springer-Verlag, New York (1992)).

A “genetically engineered” or “recombinant” cell is a cell having one or more modifications to the genetic material of the cell. Such modifications include, but are not limited to, insertions of genetic material, deletions of genetic material and insertion of genetic material that is extrachromasomal whether such material is stably maintained or not.

As used herein, the term “modified sugar.” refers to a naturally- or non-naturally-occurring carbohydrate that is enzymatically added onto an amino acid or a glycosyl residue of a peptide in a process of the disclosure. The modified sugar is selected from a number of enzyme substrates including, but not limited to, sugar nucleotides (mono-, di-, and tri-phosphates), activated sugars (e.g., glycosyl halides, glycosyl mesylates) and sugars that are neither activated nor nucleotides. A “modified sugar” maybe covalently functionalized with a “modifying group.” Useful modifying groups include, but are not limited to, water-soluble and -insoluble polymers, therapeutic moieties, diagnostic moieties, and biomolecules. The locus of functionalization with the modifying group is selected such that it does not prevent the “modified sugar” from being added enzymatically to a peptide or glycosyl residue of the peptide.

The term “zygosity status” as used herein refers to a sample, a cell population, or an organism as appearing heterozygous, homozygous, or hemizygous as determined by testing methods known in the art and described herein. The term “zygosity status of a nucleic acid” means determining whether the source of nucleic acid appears heterozygous, homozygous, or hemizygous. The “zygosity status” may refer to differences in a single nucleotide in a sequence. In some methods, the zygosity status of a sample with respect to a single mutation may be categorized as homozygous wild-type, heterozygous (i.e., one wild-type allele and one mutant allele), homozygous mutant, or hemizygous (i.e., a single copy of either the wild-type or mutant allele). Because direct sequencing of plasma or cell samples as routinely performed in clinical laboratories does not reliably distinguish between hemizygosity and homozygosity, in some embodiments, these classes are grouped. For example, samples in which no or a minimal amount of wild-type nucleic acid is detected are termed “hemizygous/homozygous mutant.” In some embodiments, a “minimal amount” may be between about 1-2%. In other embodiments, a minimal amount may be between about 1-3%. In still other embodiments, a “minimal amount” may be less than 1%.

### Treatment Methods

In part, the disclosure provides new genetics-based therapeutic regimens for treating a myeloproliferative disorder (MPD) using an SAP protein. In one aspect, the disclosure provides methods of treating an MPD in a subject carrying one or more somatic mutations or cytogenetic abnormalities associated with an MPD. In some embodiments, the method comprises administering a therapeutically effective amount of a serum amyloid P (SAP) protein to a subject carrying a mutation (e.g., the mutation may be found in some hematopoietic cells of the subject) associated with the MPD in one or more genes selected from: JAK2, MPL, CALR, ASXL1, EZH2, SRSF2, IDH1, or IDH2. The SAP proteins of the disclosure are used, alone or in combination with an additional agent, to treat an MPD. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In some embodiments, the mutation associated with the MPD is not JAK2V617F. The disclosure contemplates any of the methods described herein having any combination of features described herein.

In one aspect, the disclosure provides methods of treating an MPD in a subject carrying one or more somatic mutations and/or cytogenetic abnormalities associated with an MPD by administering an SAP protein according to a dosing schedule and/or dosage regimen effective to reduce mutant allele burden and/or cytogenetic abnormalities. In some embodiments, the method comprises administering a therapeutically effective amount of a serum amyloid P (SAP) protein to a subject carrying a mutation (e.g., the mutation may be found in some hematopoietic cells of the subject) associated with the myeloproliferative disorder in one or more genes selected from: JAK2, MPL, CALR, ASXL1, EZH2, SRSF2, IDH1, or IDH2, and wherein the SAP protein is administered according to a dosage regimen effective to reduce mutant allele burden of said gene in said subject. The SAP proteins of the disclosure are used, alone or in combination with an additional agent, to treat the MPD. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In some embodiments, the mutation associated with the MPD is not JAK2V617F.

In one aspect, the disclosure provides methods of reducing mutant allele burden and/or cytogenetic abnormalities associated with an MPD by administering an SAP protein. In some embodiments, the disclosure provides a method for reducing mutant allele burden in in one or more genes selected from: JAK2, MPL, CALR, ASXL1, EZH2, SRSF2, IDH1, or IDH2. The SAP proteins of the disclosure are used, alone or in combination with an additional agent, to treat the MPD. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In some embodiments, the mutation associated with the MPD is not JAK2V617F.

In one aspect, the disclosure provides methods of monitoring the effectiveness of an SAP protein therapy for an MPD based on quantitative and qualitative analyses of the mutational status and/or cytogenetic information of a subject receiving the SAP protein. The quantitative analysis of the mutational status may comprise measuring the mutant allele burden and may comprise a comparison of the mutational status prior to starting SAP protein therapy and at different time points during the course of therapy. Similarly, cytogenetic analyses are carried out prior to starting therapy and at different time points during the course of therapy. In some embodiments, the method comprises: (i) measuring a first mutant allele burden of a mutation in one or more genes associated with the MPD selected from: JAK2, MPL, CALR, ASXL1, EZH2, SRSF2, IDH1, or IDH2, wherein said first mutant allele burden is measured before administration of the SAP protein; (ii) measuring a second mutant allele burden of the same mutation measured in (i), wherein said second mutant allele burden is measured after administration of the SAP protein; and (iii) identifying a difference between the second mutant allele burden and the first mutant allele burden. In a further embodiment, a decrease in the second mutant allele burden relative to the first mutant allele burden indicates that the administration of the SAP protein is effective in treating the MPD and the dosage regimen may be maintained or modified to decrease the dosage and/or frequency of administration. The SAP proteins of the disclosure are used, alone or in combination with an additional agent, to treat the MPD. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In some embodiments, the mutation associated with the myeloproliferative disorder is not JAK2V617F.

In one aspect, the disclosure provides methods of determining responsiveness to SAP protein or agonist therapy based on the presence or absence of one or more somatic mutations and/or cytogenetic abnormalities associated with an MPD. In some embodiments, the method comprises (i) determining whether the cells of a subject having a myeloproliferative disorder carry a mutation associated with the myeloproliferative disorder in one or more genes selected from: JAK2, MPL, CALR, ASXL1, EZH2, SRSF2, IDH1, or IDH2; and if the subject carries said mutant allele (ii) administering a therapeutically effective amount of a serum amyloid P (SAP) protein to the subject. The SAP proteins of the disclosure are used, alone or in combination with an additional agent, to treat the MPD. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In some embodiments, the mutation associated with the MPD is not JAK2V617F. In some embodiments of any of the above aspects of the disclosure, the subject comprises a mutation in one or more genes selected from TET2, CBL, IKZF1, LNK, DNMT3A, CUX1, U2AF1, and SF3B1 and/or the methods of the disclosure further comprise carrying out mutational analyses of one or more genes selected from TET2, CBL, IKZF1, LNK, DNMT3A, CUX1, U2AF1, and SF3B1.

Any of the above aspects of the disclosure can be further combined with cytogenetic analyses to assay for one or more of MPD-associated cytogenetic abnormalities such as, but not limited to, monosomal karyotype, inv(3), i(17q), −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities. Tefferi et al. ASH 2014 Abstract 631. In some embodiments, cytogenetic analysis is carried out according to the International System for Human Cytogenetic Nomenclature (Cytogenetic and genome research. 2013. Prepublished on 2013 Jul. 3 as DOI 10.1159/000353118). In some embodiments, assignment to “normal” karyotype requires a minimum of 10 metaphases analyzed. In some embodiments, a complex karyotype is defined as the presence of 3 or more distinct structural or numeric abnormalities. In some embodiments, a monosomal karyotype is defined as 2 or more distinct autosomal monosomies or single autosomal monosomy associated with at least one structural abnormality (JCO. 2008; 26:4791; Tefferi et al. ASH 2014 Abstract 631).

Myeloproliferative disease (MPD) refers to a group of disorders characterized by clonal abnormalities of the hematopoietic cells leading to excess production of various blood cells in the bone marrow. Since the hematopoietic stem cell gives rise to myeloid, erythroid, and platelet cells, qualitative and quantitative changes can be seen in any or all of these cell lines. Myeloproliferative disorders can be challenging to diagnose and treat. The term “myeloproliferative disorder (MPD)” or “myeloproliferative disease” is meant to include non-lymphoid dysplastic or neoplastic conditions arising from a hematopoietic stem cell or its progeny. “MPD patient” includes a patient who has been diagnosed with an MPD. “Myeloproliferative disease” is meant to encompass the specific, classified types of myeloproliferative diseases including polycythemia vera (PV), essential thrombocythemia (ET) and idiopathic myelofibrosis (IMF) or primary myelofibrosis (PMF). Also included in the definition are hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, and systemic mastocytosis (SM). “Myeloproliferative disorder” is also meant to encompass any unclassified myeloproliferative diseases (UMPD or MPD-NC).

In certain aspects, the disclosure encompasses the use of an SAP protein, as a single agent or in combination with another agent, for a genetics-based treatment of myelofibrosis. Myelofibrosis (“MF”) is a BCR-ABL1-negative mycloproliferative neoplasm (“MPN”) that presents de novo (primary) or may be preceded by polycythemia vera (“PV”) or essential thrombocythemia (“ET”). Primary myelofibrosis (PMF) (also referred to in the literature as idiopathic myeloid metaplasia, and Agnogenic myeloid metaplasia) is a clonal disorder of multipotent hematopoietic progenitor cells of monocytic lineage (reviewed in Abdel-Wahab, O. et al. (2009) Annu. Rev. Med. 60:233-45; Varicchio, L. et al. (2009) Expert Rev. Hematol. 2(3):315-334; Agrawal, M. et al. (2011) Cancer 117(4):662-76). Myelofibrosis originates from acquired mutations that target the hematopoietic stem cell and induce dysregulation of kinase signaling, clonal myeloproliferation, and abnormal cytokine expression (Tefferi. Blood 2011, 117(13): 3494-3504). The disease is characterized by anemia, splenomegaly and extramedullary hematopoiesis, and is marked by progressive marrow fibrosis and atypical megakaryocytic hyperplasia. CD34+ stem/progenitor cells abnormally traffic in the peripheral blood and multi organ extramedullary erythropoiesis is a hallmark of the disease, especially in the spleen and liver. The bone marrow structure is altered due to progressive fibrosis, neoangiogenesis, and increased bone deposits. Median survival ranges from less than 2 years to over 15 years based on currently identified prognostic factors (Cervantes F et al., Blood 113:2895-2901, 2009; Hussein K et al. Blood 115:496-499, 2010: Patnaik M M et al., Eur J Haematol 84:105-108, 2010).

It is known in the literature that inhibitors of JAK2 are useful in the treatment and/or prevention of MPDs. See, e.g., Tefferi, A, and Gilliland, D. G. Mayo Clin. Proc. 80(7): 947-958 (2005); Fernandez-Luna, J. L. et al. Haematologica 83(2): 97-98 (1998); Harrison, C. N. Br. J. Haematol. 130(2): 153-165 (2005); Leukemia (2005) 19, 1843-1844; and Tefferi, A. and Barbui, T. Mayo Clin. Proc. 80(9): 1220-1232 (2005). However, the management options of MF are currently inadequate to meet the needs of all patients. Therefore, there is a need to provide additional therapy options for MF patients, such as those provided herein.

In some embodiments of the methods provided herein, the subject has primary myelofibrosis. In some embodiments of the compositions and methods provided herein, the subject has post polycythemia vera myelofibrosis (post-PV MF). In some embodiments, the subject has post essential thrombocythemia myelofibrosis (post-ET MF). In some embodiments, the subject has high risk myelofibrosis. In some embodiments, the subject has intermediate risk myelofibrosis (such as intermediate risk level 1 or intermediate risk level 2). In some embodiments, the subject has low risk myelofibrosis. In some embodiments, the subject has PV or ET without fibrosis. In some embodiments, the subject is positive (e.g., the mutation is present) for the valine 617 to phenylalanine mutation of human Janus Kinase 2 (JAK2) or positive for the mutation corresponding to the valine 617 to phenylalanine mutation of human JAK2. In some embodiments, the subject is negative (e.g., the mutation is absent) for the valine 617 to phenylalanine mutation of human Janus Kinase 2 (JAK2) or negative for the mutation corresponding to the valine 617 to phenylalanine mutation of human JAK2. In some embodiments, the subject has a mutation in exon 12 or exon 14 of JAK2. In some embodiments, the subject has a mutation at codon 515 of MPL. In some embodiments, the subject has a W515L, W515K, W515A, or W515R amino acid substitution in MPL. In some embodiments, the subject has a mutation in exon 10 of MPL. In some embodiments, the subject has a mutation in exon 9 of CALR. In some embodiments, the subject has a mutation in exon 12 of ASXL1. In some embodiments, the subject has a mutation in exon 4 of IDH1. In some embodiments, the subject has a mutation at codon 132 of IDH1. In some embodiments, the subject has a mutation in exon 4 of IDH2. In some embodiments, the subject has a mutation at codon 140 of IDH2. In some embodiments, the subject has a mutation at codon 172 of IDH2. In some embodiments, prior to initiation of treatment with an SAP protein of the disclosure, the subject has bone marrow fibrosis and the fibrosis is measurable according to the grading system of the European Consensus on Grading of Bone Marrow Fibrosis. In some embodiments, prior to initiation of treatment with an SAP protein of the disclosure, the subject has bone marrow fibrosis of greater than or equal to Grade 2. In other embodiments, prior to initiation of treatment with an SAP protein of the disclosure, the subject has bone marrow fibrosis of Grade 3. In other embodiments, prior to initiation of treatment with an SAP protein of the disclosure, the subject has bone marrow fibrosis of Grade 1.

In certain embodiments, the fibrotic condition of the bone marrow is an intrinsic feature of a chronic myeloproliferative neoplasm of the bone marrow, such as primary myelofibrosis. In other embodiments, the bone marrow fibrosis is associated with a malignant+−condition or a condition caused by a clonal proliferative disease or a hematologic disorder such as but not limited to hairy cell leukemia, lymphoma (e.g., Hodgkin or non-Hodgkin lymphoma), multiple myeloma or chronic myelogenous leukemia (CML). In yet other embodiments, the bone marrow fibrosis is associated with a solid tumor metastasis to the bone marrow.

In certain embodiments, the SAP proteins of the disclosure (e.g., an SAP protein comprising a glycosylated SAP protein; recombinant human SAP protein; etc.) are used to treat myelofibrosis by decreasing fibrosis to restore organ function. Administering an SAP protein of the disclosure as a single agent or as part of a combination therapy, resulted in a decrease in organ fibrosis (e.g. bone marrow fibrosis), leading to improvements and/or restoration of organ function, improvement in hemoglobin, improvement in blood counts such as platelets or white blood cells, or improvement in symptoms. Improvement in organ function can be evaluated for example, by assessing improvement in platelet levels and/or hemoglobin in the subject over the course of treatment, such as over 12, 20, 24, or greater than 24 weeks of treatment (e.g., greater than 30 weeks, greater than 36 weeks, greater than 42 weeks, greater than 48 weeks).

As described herein, in certain embodiments, an SAP protein is used as a monotherapy in a subject who has a mutation in one or more MPD-associated genes (e.g., JAK2, MPL, CALR, ASXL1, EZH2, SRSF2, IDH1, or IDH2) or one or more MPD-associated cytogenetic abnormalities. Optionally, the subject is characterized based on other features (e.g., level of manifestations, such as initial level of bone marrow fibrosis or other symptoms). In some embodiments, the subject is positive (e.g., the mutation is present) for the valine 617 to phenylalanine mutation of human Janus Kinase 2 (JAK2) or positive for the mutation corresponding to the valine 617 to phenylalanine mutation of human JAK2. In some embodiments, the subject is negative (e.g., the mutation is absent) for the valine 617 to phenylalanine mutation of human Janus Kinase 2 (JAK2) or negative for the mutation corresponding to the valine 617 to phenylalanine mutation of human JAK2. In some embodiments, the subject has a mutation in exon 12 or exon 14 of JAK2. In some embodiments, the subject has a mutation at codon 515 of MPL. In some embodiments, the subject has a W515L, W515K, W515A, or W515R amino acid substitution in MPL. In some embodiments, the subject has a mutation in exon 10 of MPL. In some embodiments, the subject has a mutation in exon 9 of CALR. In some embodiments, the subject has a mutation in exon 12 of ASXL1. In some embodiments, the subject has a mutation in exon 4 of IDH1. In some embodiments, the subject has a mutation at codon 132 of IDH1. In some embodiments, the subject has a mutation in exon 4 of IDH2. In some embodiments, the subject has a mutation at codon 140 of IDH2. In some embodiments, the subject has a mutation at codon 172 of IDH2. In some embodiments, the mutation associated with the MPD is not JAK2V617F. In some embodiments, the subject has one or more mutations in one or more genes selected from TET2, CBL, IKZF1, LNK, DNMT3A, CUX1, U2AF1, and SF3B1. In some embodiments, the subject has one or more cytogenetic abnormalities selected from monosomal karyotype, inv(3), i(17q), −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities.

As described herein, in certain embodiments, addition of SAP to a therapeutic regimen or replacement of a therapeutic regimen with SAP therapy is used in a subject who is unresponsive, resistant or otherwise refractory to a treatment (in the absence of the SAP) or for whom efficacy of the treatment is or has waned. In certain embodiments, the addition of or substitution with SAP is used to expand the patient population for which treatment with another therapeutic agent is suitable (e.g., SAP expands the therapeutic window or patient population for another drug). In some embodiments, the patients are intolerant of a treatment or ineligible for it (e.g. ruxolitinib therapy in the absence of the SAP). By way of example, certain cancers are known to be unresponsive to chemotherapy. Without being bound by theory, fibrosis may hinder effective access of the drugs to the tumor. In some embodiments, the subject is positive (e.g., the mutation is present) for the valine 617 to phenylalanine mutation of human Janus Kinase 2 (JAK2) or positive for the mutation corresponding to the valine 617 to phenylalanine mutation of human JAK2. In some embodiments, the subject is negative (e.g., the mutation is absent) for the valine 617 to phenylalanine mutation of human Janus Kinase 2 (JAK2) or negative for the mutation corresponding to the valine 617 to phenylalanine mutation of human JAK2. In some embodiments, the subject has a mutation in exon 12 or exon 14 of JAK2. In some embodiments, the subject has a mutation at codon 515 of MPL. In some embodiments, the subject has a W515L, W515K, W515A, or W515R amino acid substitution in MPL. In some embodiments, the subject has a mutation in exon 10 of MPL. In some embodiments, the subject has a mutation in exon 9 of CALR. In some embodiments, the subject has a mutation in exon 12 of ASXL1. In some embodiments, the subject has a mutation in exon 4 of IDH1. In some embodiments, the subject has a mutation at codon 132 of IDH1. In some embodiments, the subject has a mutation in exon 4 of IDH2. In some embodiments, the subject has a mutation at codon 140 of IDH2. In some embodiments, the subject has a mutation at codon 172 of IDH2. In some embodiments, the mutation associated with the MPD is not JAK2V617F. In some embodiments, the subject has one or more mutations in one or more genes selected from TET2, CBL, IKZF1, LNK, DNMT3A, CUX1, U2AF1, and SF3B1. In some embodiments, the subject has one or more cytogenetic abnormalities selected from monosomal karyotype, inv(3), i(17q), −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities.

In certain embodiments, an SAP protein is used as a monotherapy and/or is used to treat naïve patients. In certain embodiments, an SAP protein is used in patients whose disease has a certain fibrotic score, such as bone marrow fibrosis of Grade 2 or Grade 3, as assessed by the European Consensus on Grading of Bone Marrow Fibrosis. In some embodiments, the subject is positive (e.g., the mutation is present) for the valine 617 to phenylalanine mutation of human Janus Kinase 2 (JAK2) or positive for the mutation corresponding to the valine 617 to phenylalanine mutation of human JAK2. In some embodiments, the subject is negative (e.g., the mutation is absent) for the valine 617 to phenylalanine mutation of human Janus Kinase 2 (JAK2) or negative for the mutation corresponding to the valine 617 to phenylalanine mutation of human JAK2. In some embodiments, the subject has a mutation in exon 12 or exon 14 of JAK2. In some embodiments, the subject has a mutation at codon 515 of MPL. In some embodiments, the subject has a W515L, W515K, W515A, or W515R amino acid substitution in MPL. In some embodiments, the subject has a mutation in exon 10 of MPL. In some embodiments, the subject has a mutation in exon 9 of CALR. In some embodiments, the subject has a mutation in exon 12 of ASXL1. In some embodiments, the subject has a mutation in exon 4 of IDH1. In some embodiments, the subject has a mutation at codon 132 of IDH1. In some embodiments, the subject has a mutation in exon 4 of IDH2. In some embodiments, the subject has a mutation at codon 140 of IDH2. In some embodiments, the subject has a mutation at codon 172 of IDH2. In some embodiments, the mutation associated with the MPD is not JAK2V617F. In some embodiments, the subject has one or more mutations in one or more genes selected from TET2, CBL, IKZF1, LNK, DNMT3A, CUX1, U2AF1, and SF3B1. In some embodiments, the subject has one or more cytogenetic abnormalities selected from monosomal karyotype, inv(3), i(17q), −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities.

In certain embodiments, an SAP protein is used as a monotherapy and/or is used to treat patients who are anemic or thrombocytopenic. In some embodiments, the subject is positive (e.g., the mutation is present) for the valine 617 to phenylalanine mutation of human Janus Kinase 2 (JAK2) or positive for the mutation corresponding to the valine 617 to phenylalanine mutation of human JAK2. In some embodiments, the subject is negative (e.g., the mutation is absent) for the valine 617 to phenylalanine mutation of human Janus Kinase 2 (JAK2) or negative for the mutation corresponding to the valine 617 to phenylalanine mutation of human JAK2. In some embodiments, the subject has a mutation in exon 12 or exon 14 of JAK2. In some embodiments, the subject has a mutation at codon 515 of MPL. In some embodiments, the subject has a W515L, W515K, W515A, or W515R amino acid substitution in MPL. In some embodiments, the subject has a mutation in exon 10 of MPL. In some embodiments, the subject has a mutation in exon 9 of CALR. In some embodiments, the subject has a mutation in exon 12 of ASXL1. In some embodiments, the subject has a mutation in exon 4 of IDH1. In some embodiments, the subject has a mutation at codon 132 of IDH1. In some embodiments, the subject has a mutation in exon 4 of IDH2. In some embodiments, the subject has a mutation at codon 140 of IDH2. In some embodiments, the subject has a mutation at codon 172 of IDH2. In some embodiments, the mutation associated with the MPD is not JAK2V617F. In some embodiments, the subject has one or more mutations in one or more genes selected from TET2, CBL, IKZF1, LNK, DNMT3A, CUX1, U2AF1, and SF3B1. In some embodiments, the subject has one or more cytogenetic abnormalities selected from monosomal karyotype, inv(3), i(17q). −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities.

In certain aspects, the myeloproliferative disease is polycythemia vera, essential thrombocythemia, myelofibrosis, or an unclassified myeloproliferative disease. In some embodiments, the myelofibrosis is primary myleofibrosis, post-PV myelofibrosis, or post-ET myelofibrosis.

In any of the above aspects, an SAP protein of the disclosure (such as a recombinant human SAP protein, such as a glycosylated SAP protein) may be used in combination with any of the additional therapeutic agents described herein. In some embodiments, the additional therapeutic agent is an anti-cancer agent.

Exemplary Mutations Associated with Myeloproliferative Disorders JAK2

Janus kinase 2 (JAK2) is a non-receptor tyrosine kinase and acts as an intermediary between membrane-bound cytokine receptors, and down-stream members of the signal transduction pathway such as STAT (Signal Transducers and Activators of Transcription protein) molecules which then act as transcription factors in the nucleus. It has long been hypothesized that perturbation of protein tyrosine kinase (PTK) signaling by mutations and other genetic alterations is associated with MPDs. Mutant PTKs such as, for example. Janus kinase 2 (JAK2) gene mutations, can lead to constitutive activity in patients with MPDs or to other defects. As such, in certain embodiments, the subject being treated has a mutation in JAK2, such as a mutation described herein and/or the subject is evaluated for allele burden prior to and/or during treatment.

The JAK2 V617F substitution relieves the auto-inhibition of its kinase activity, leading to a constitutively active kinase and augments downstream JAK2-STAT signaling pathways (see e.g., Saharinen et al. Mol Cell Biol. 2000, 20:3387-3395; Saharinen et al., Mol Biol Cell 2003, 14(4): 1448-1459). The mutation has been detected from blood samples, bone marrow and buccal samples and contributes to the pathogenesis of MPD (see. e.g., Baxter et al. Lancet 2005, 365:1054-1060; James et al. Nature 2005, 438: 1144-1148; Zhao et al. J. Biol. Chem. 2005, 280(24):22788-22792; Levine et al. Cancer Cell 2005, 7:387-397; Kralovics et al. New Eng. J. Med. 2005, 352(17): 1779-1790), and homozygous and heterozygous cell populations have been reported in MPD patients (Baxter et al., Lancet 2005, 365:1054-1060). Other JAK2 mutations in humans including translocations, point mutations, deletions, and insertions have been reported. See e.g., Scott et al., N Engl J Med. 2007, 356:459-468; Li et al. Blood 2008, 111:3863-3866.

Over 10 different sequence variations, mostly occurring between codons 536 and 544, and involving a deletion of three to six nucleotides have been found in exon 12 of JAK2. Some duplications and some 2-bp replacements have also been found (Laughlin et al. J Mol Diagn. 2010, 12(3): 278-282).

Other exemplary mutations in JAK2 include: exon 12 missense mutations such as T514M, N533Y, L545V, F547L; exon 13 missense mutations such as F556V. R564L, R564Q, V567L, V567A, G571S, G571R, L579F, H587N, S591L, exon 14 missense mutation H606Q, exon 14 deletion S593-N622; exon 15 missense mutations L624P, I645V (see, e.g., U.S. Patent Application Publication No. 2010/0112571); K539L, V617I, C618R, L624P, whole exon 14-deletion (Lee et al. BMC Struct Biol. 2009, 9:58).

JAK2 genomic nucleic acid is located in human chromosome 9. An exemplary sequence of all or portions of human JAK2 mRNA includes but is not limited to GenBank Accession number NM_004972 (SEQ ID NO: 5). These sequences are incorporated herein by reference.

For the JAK2 nucleic acid sequence, a “mutation” means a JAK2 nucleic acid sequence that includes at least one nucleic acid variation as compared to reference sequence GenBank accession number NM_004972 (SEQ ID NO: 5). A mutation in JAK2 nucleic acid may result in a change in the encoded polypeptide sequence or the mutation may be silent with respect to the encoded polypeptide sequence. A change in an amino acid sequence may be determined as compared to NP_004963 (SEQ ID NO: 6) as a reference amino acid sequence.

In some embodiments, the JAK2 mutation is a missense mutation, a deletion mutation, an insertion mutation or a translocation. In some embodiments, mutations in JAK2 include exon 12 mutations or exon 14 mutations. In some embodiments the JAK2 mutation is JAK2V617F. In some embodiments, the JAK2 mutation is T514M, N533Y, L545V, F547L, F556V, R564L, R564Q, V567L. V567A, G571S, G571R, L579F, H587N, S591L, H606Q, L624P, 1645V, K539L, V617I, C618R, L624P, exon 14 deletion S593-N622, or whole exon 14-deletion.

In some embodiments, the disclosure provides a method of treating an MPD comprising administering a therapeutically effective amount of an SAP protein to a subject carrying a mutation in JAK2 (e.g., some of the hematopoietic cells of the subject carry a JAK2 mutation). In some embodiments, the disclosure provides a method of treating an MPD comprising administering a therapeutically effective amount of an SAP protein to a subject carrying a mutation (e.g., the mutation may be found in some hematopoietic cells of the subject) in JAK2, according to a dosage regimen effective to reduce mutant JAK2 allele burden in said subject. In some embodiments, the disclosure provides a method for reducing mutant allele burden in JAK2 in a subject suffering from a myeloproliferative disorder. In some embodiments, the disclosure provides methods of monitoring the effectiveness of an SAP protein therapy for an MPD based on JAK2 mutational status. In one embodiment, the method comprises: (i) measuring a first mutant allele burden of a mutation in JAK2, wherein said first mutant allele burden is measured before administration of the SAP protein; (ii) measuring a second mutant allele burden of the same mutation measured in (i), wherein said second mutant allele burden is measured after administration of the SAP protein; and (iii) identifying a difference between the second mutant allele burden and the first mutant allele burden. In a further embodiment, a decrease in the second mutant allele burden relative to the first mutant allele burden indicates that the administration of the SAP protein is effective in treating the myeloproliferative disorder and the dosage regimen may be maintained or modified to decrease the dosage and/or frequency of administration. In an alternative embodiment, if there is no change in the second mutant allele burden relative to the first mutant allele burden, the dosage regimen may be modified to increase the dosage and/or frequency of administration. In one aspect, the disclosure provides methods of determining responsiveness to SAP protein or agonist therapy based on the presence or absence of one or more somatic mutations in JAK2. In some embodiments, the method comprises (i) determining whether the cells of a subject having a mycloproliferative disorder carry a mutation associated with the myeloproliferative disorder in JAK2; and if the subject carries said mutant allele (ii) administering a therapeutically effective amount of an SAP protein to the subject. In one aspect, the disclosure provides a method of using a JAK2 mutation as a prognostic marker for measuring response to treatment with an SAP protein of the disclosure. In one embodiment, the method comprises: (i) measuring a first mutant allele burden of a mutation in JAK2, wherein said first mutant allele burden is measured before administration of the SAP protein; (ii) measuring a second mutant allele burden of the same mutation measured in (i), wherein said second mutant allele burden is measured after administration of the SAP protein; and (iii) measuring the difference between the second mutant allele burden and the first mutant allele burden. In a further embodiment, a decrease in the second mutant allele burden relative to the first mutant allele burden indicates a positive prognosis. In an alternative embodiment, if there is no change in the second mutant allele burden relative to the first mutant allele burden indicates a neutral or negative prognosis. In some embodiments, the JAK2 mutation is a missense mutation, a deletion mutation, an insertion mutation or a translocation. In some embodiments, mutations in JAK2 include exon 12 mutations or exon 14 mutations. In some embodiments the JAK2 mutation is JAK2V617F. In some embodiments, the JAK2 mutation is T514M. N533Y, L545V, F547L, F556V, R564L, R564Q, V567L, V567A, G571S, G571R, L579F, H587N. S591L, H606Q, L624P, I645V, K539L, V617I, C618R, L624P, exon 14 deletion S593-N622, or whole exon 14-deletion. The SAP proteins of the disclosure (such as a recombinant human SAP protein, such as a glycosylated SAP protein) are used, alone or in combination with an additional agent, to treat a mycloproliferative disorder. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In some embodiments, the mutation associated with the myeloproliferative disorder is not JAK2V617F. In certain embodiments, any of the foregoing methods further comprise assaying for one or more mutations in one or more MPD-associated genes such as but not limited to, MPL, CALR, ASXL1, EZH2, SRSF2, IDH1, or IDH2. In some embodiments, any of the foregoing methods further comprise assaying for one or more mutations in TET2, CBL, IKZF1, LNK, DNMT3A, CUX1, U2AF1, or SF3B1. In some embodiments, any of the foregoing methods further comprise assaying for one or more cytogenetic abnormalities such as monosomal karyotype, inv(3), i(17q), −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities. In some embodiments of any of the above aspects of the disclosure, the MPD is polycythemia vera, essential thrombocythemia myelofibrosis, or an unclassified myeloproliferative disease. In some embodiments, the myelofibrosis is primary myleofibrosis, post-PV myelofibrosis, or post-ET myelofibrosis.

Methods of the disclosure involve evaluating a sample containing nucleic acids form an individual having or suspected of having an MPD for the presence or absence of JAK2 mutations. The sample may be any suitable biological sample including, for example, whole blood (e.g., JAK2 nucleic acid being extracted from the cellular fraction), plasma, serum, bone marrow, and tissue samples (e.g., biopsy and paraffin-embedded tissue). The JAK2 nucleic acid may be any convenient nucleic acid type including, for example, genomic DNA, RNA (e.g., mRNA), or cDNA prepared from subject RNA. Alternatively, the JAK2 nucleic acid mutation may be inferred by assessing the JAK2 protein from the individual. For example, identification of a mutant JAK2 protein is indicative of a mutation in the JAK2 gene. Suitable detection methodologies include oligonucleotide probe hybridization, primer extension reaction, nucleic acid sequencing, and protein sequencing. In some embodiments, the individual is screened for the presence of other pathological mutations in one or more additional MPD-associated genes (e.g., MPL, CALR, ASXL1, EZH2, SRSF2, IDH1, or IDH2) either simultaneously or prior to screening for the JAK2 nucleic acid mutation. In some embodiments, one or more (e.g., 2, 3, 4, 5, 6, 7, or 8) mutations in addition to a JAK2 mutation are used as a prognostic marker for measuring response to treatment with an SAP protein of the disclosure.

### MPL

Myeloproliferative leukemia protein (MPL) is the receptor for thrombopoietin that regulates the production of platelets by bone marrow. Recently, acquired mutations in the transmembrane-juxtamembrane region of MPL (MPLW515 mutations) have been reported in approximately 5% of JAK2V617F-negative PMF and about 1% of all cases of ET (Pardanani et al. Blood. 2006, 108(10): 3472-3476 and Pikman et al. PLoS Med. 2006, 3:e270). Like JAK2V617F, the MPL mutations confer constitutive activation of the JAK-STAT pathway.

Other exemplary MPL mutations include mutations in the MPL nucleic acid such as deletion/insertion mutations in exons 10 and 11 described in U.S. Patent Application Publication No. 2013/0053262.

MPL genomic nucleic acid is located in human chromosome 1. An exemplary sequence of all or portions of human MPL mRNA includes but is not limited to GenBank Accession number NM_005373 (SEQ ID NO: 7). These sequences are incorporated herein by reference.

For the MPL nucleic acid sequence, a “mutation” means a MPL nucleic acid sequence that includes at least one nucleic acid variation as compared to reference sequence GenBank accession number NM 005373 (SEQ ID NO: 7). A mutation in MPL nucleic acid may result in a change in the encoded polypeptide sequence or the mutation may be silent with respect to the encoded polypeptide sequence. A change in an amino acid sequence may be determined as compared to NP_005364 (SEQ ID NO: 8) as a reference amino acid sequence.

In some embodiments, the MPL mutation is a missense mutation, a deletion mutation, an insertion mutation or a translocation. In some embodiments, mutations in MPL include insertion/deletion mutations in exon 10 of MPL. In some embodiments, mutations in MPL include insertion/deletion mutations in exon 11 of MPL. In some embodiments, the mutation in MPL includes a mutation in codon 515. In some embodiments, mutations in MPL include MPLW515L, MPLW515K, MPLW515A or MPLW515R.

In some embodiments, the disclosure provides a method of treating an MPD comprising administering a therapeutically effective amount of an SAP protein to a subject carrying a mutation in MPL (e.g., some of the hematopoietic cells of the subject carry a MPL mutation). In some embodiments, the disclosure provides a method of treating an MPD comprising administering a therapeutically effective amount of an SAP protein to a subject carrying a mutation (e.g., the mutation may be found in some hematopoietic cells of the subject) in MPL, according to a dosage regimen effective to reduce mutant MPL allele burden in said subject. In some embodiments, the disclosure provides a method for reducing mutant allele burden in MPL in a subject suffering from a myeloproliferative disorder. In some embodiments, the disclosure provides methods of monitoring the effectiveness of an SAP protein therapy for an MPD based on MPL mutational status. In one embodiment, the method comprises: (i) measuring a first mutant allele burden of a mutation in MPL, wherein said first mutant allele burden is measured before administration of the SAP protein; (ii) measuring a second mutant allele burden of the same mutation measured in (i), wherein said second mutant allele burden is measured after administration of the SAP protein; and (iii) identifying a difference between the second mutant allele burden and the first mutant allele burden. In a further embodiment, a decrease in the second mutant allele burden relative to the first mutant allele burden indicates that the administration of the SAP protein is effective in treating the myeloproliferative disorder and the dosage regimen may be maintained or modified to decrease the dosage and/or frequency of administration. In an alternative embodiment, if there is no change in the second mutant allele burden relative to the first mutant allele burden, the dosage regimen may be modified to increase the dosage and/or frequency of administration. In one aspect, the disclosure provides methods of determining responsiveness to SAP protein therapy based on the presence or absence of one or more somatic mutations in MPL. In some embodiments, the method comprises (i) determining whether the cells of a subject having a myeloproliferative disorder carry a mutation associated with the mycloproliferative disorder in MPL; and if the subject carries said mutant allele (ii) administering a therapeutically effective amount of an SAP protein to the subject. In one aspect, the disclosure provides a method of using an MPL mutation as a prognostic marker for measuring response to treatment with an SAP protein of the disclosure. In one embodiment, the method comprises: (i) measuring a first mutant allele burden of a mutation in MPL, wherein said first mutant allele burden is measured before administration of the SAP protein; (ii) measuring a second mutant allele burden of the same mutation measured in (i), wherein said second mutant allele burden is measured after administration of the SAP protein; and (iii) measuring the difference between the second mutant allele burden and the first mutant allele burden. In a further embodiment, a decrease in the second mutant allele burden relative to the first mutant allele burden indicates a positive prognosis. In an alternative embodiment, if there is no change in the second mutant allele burden relative to the first mutant allele burden indicates a neutral or negative prognosis. In some embodiments, the MPL mutation is a missense mutation, a deletion mutation, an insertion mutation or a translocation. In some embodiments, mutations in MPL include insertion/deletion mutations in exon 10 of MPL. In some embodiments, mutations in MPL include insertion/deletion mutations in exon 11 of MPL. In some embodiments, the mutation in MPL includes a mutation in codon 515. In some embodiments, mutations in MPL include MPLW515L, MPLW515K, MPLW515A or MPLW515R. The SAP proteins of the disclosure (such as a recombinant human SAP protein, such as a glycosylated SAP protein) are used, alone or in combination with an additional agent, to treat a myeloproliferative disorder. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In certain embodiments, any of the foregoing methods further comprise assaying for one or more mutations in one or more MPD-associated genes such as but not limited to, JAK2, CALR, ASXL1, EZH2, SRSF2, IDH1, or IDH2. In some embodiments, any of the foregoing methods further comprise assaying for one or more mutations in TET2, CBL, IKZF1, LNK, DNMT3A, CUX1, U2AF1, or SF3B1. In some embodiments, any of the foregoing methods further comprise assaying for one or more cytogenetic abnormalities such as monosomal karyotype, inv(3), i(17q), −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities. In some embodiments of any of the above aspects of the disclosure, the MPD is polycythemia vera, essential thrombocythemia, myelofibrosis, or an unclassified myeloproliferative disease. In some embodiments, the myelofibrosis is primary myleofibrosis, post-PV myelofibrosis, or post-ET myelofibrosis.

Methods of the disclosure involve evaluating a sample containing nucleic acids from an individual having or suspected of having an MPD for the presence or absence of MPL mutations. The sample may be any suitable biological sample including, for example, whole blood (i.e., MPL nucleic acid being extracted from the cellular fraction), plasma, serum, bone marrow, and tissue samples (e.g., biopsy and paraffin-embedded tissue). The MPL nucleic acid may be any convenient nucleic acid type including, for example, genomic DNA, RNA (e.g., mRNA), or cDNA prepared from subject RNA. Alternatively, the MPL nucleic acid mutation may be inferred by assessing the MPL protein from the individual. For example, identification of a mutant MPL protein is indicative of a mutation in the MPL gene. Suitable detection methodologies include oligonucleotide probe hybridization, primer extension reaction, nucleic acid sequencing, and protein sequencing. In some embodiments, the individual is screened for the presence of other pathological mutations in one or more additional myeloproliferative disorder-associated genes (e.g., JAK2, CALR, ASXL1, EZH2, SRSF2, IDH1, or IDH2) either simultaneously or prior to screening for the MPL nucleic acid mutation. In some embodiments, one or more (e.g., 2, 3, 4, 5, 6, 7, or 8) mutations in addition to an MPL mutation are used as a prognostic marker for measuring response to treatment with an SAP protein of the disclosure.

### CALR

Calreticulin (CALR) is a highly conserved, multifunctional endoplasmic reticulum (ER) protein and plays an integral role in calcium homeostasis and protein folding inside the ER. Outside the ER, CALR regulates various integrin-mediated cell adhesion, gene nuclear transport, programmed cell removal, and immunogenic cell death. CALR encoding CALR protein is located on chromosome 19p 13.2, contains 9 exons, and spans a 4.2-kb region. Somatic insertions or deletions in exon 9 of CALR were found in as high as 70% to 84% of samples of myeloproliferative neoplasms with nonmutated JAK2 (Klampfl et al. N Engl J Med. 2013, 369(25):2379-90 and Nangalia et al. N Engl J Med. 2013, 369(25):2391-2405). The CALR mutations cause a frameshift resulting in a novel C-terminus containing a number of positively charged amino acids whereas the wildtype C-terminus is mostly negatively charged.

In essential thrombocythemia and primary myelofibrosis, CALR mutations and JAK2 and MPL mutations were mutually exclusive (Klampfl t al. N Engl J Med. 2013, 369(25):2379-90). CALR mutations are a useful diagnostic marker for JAK2/MPL-negative ET or PMF patients due to their relative high frequency. Moreover, the phenotypic manifestations are different from those of JAK2 mutations.

CALR genomic nucleic acid is located in human chromosome 19. An exemplary sequence of all or portions of human CALR mRNA includes but is not limited to GenBank Accession number NM_004343 (SEQ ID NO: 9). These sequences are incorporated herein by reference.

For the CALR nucleic acid sequence, a “mutation” means a CALR nucleic acid sequence that includes at least one nucleic acid variation as compared to reference sequence GenBank accession number NM_004343 (SEQ ID NO: 9). A mutation in CALR nucleic acid may result in a change in the encoded polypeptide sequence or the mutation may be silent with respect to the encoded polypeptide sequence. A change in an amino acid sequence may be determined as compared to NP_004334 (SEQ ID NO: 10) as a reference amino acid sequence.

In some embodiments, the CALR mutation is a missense mutation, a deletion mutation, an insertion mutation or a translocation. In some embodiments, mutations in CALR include insertion/deletion mutations in exon 9 of CALR.

In some embodiments, the disclosure provides a method of treating an MPD comprising administering a therapeutically effective amount of an SAP protein to a subject carrying a mutation in CALR (e.g., some of the hematopoietic cells of the subject carry a CALR mutation). In some embodiments, the disclosure provides a method of treating an MPD comprising administering a therapeutically effective amount of an SAP protein to a subject carrying a mutation (e.g., the mutation may be found in some hematopoietic cells of the subject) in CALR, according to a dosage regimen effective to reduce mutant CALR allele burden in said subject. In some embodiments, the disclosure provides a method for reducing mutant allele burden in CALR in a subject suffering from a myeloproliferative disorder. In some embodiments, the disclosure provides methods of monitoring the effectiveness of an SAP protein therapy for an MPD based on CALR mutational status. In one embodiment, the method comprises: (i) measuring a first mutant allele burden of a mutation in CALR, wherein said first mutant allele burden is measured before administration of the SAP protein; (ii) measuring a second mutant allele burden of the same mutation measured in (i), wherein said second mutant allele burden is measured after administration of the SAP protein; and (iii) identifying a difference between the second mutant allele burden and the first mutant allele burden. In a further embodiment, a decrease in the second mutant allele burden relative to the first mutant allele burden indicates that the administration of the SAP protein is effective in treating the myeloproliferative disorder and the dosage regimen may be maintained or modified to decrease the dosage and/or frequency of administration. In an alternative embodiment, if there is no change in the second mutant allele burden relative to the first mutant allele burden, the dosage regimen may be modified to increase the dosage and/or frequency of administration. In one aspect, the disclosure provides methods of determining responsiveness to SAP protein therapy based on the presence or absence of one or more somatic mutations in CALR. In some embodiments, the method comprises (i) determining whether the cells of a subject having a myeloproliferative disorder carry a mutation associated with the myeloproliferative disorder in CALR; and if the subject carries said mutant allele (ii) administering a therapeutically effective amount of an SAP protein to the subject. In one aspect, the disclosure provides a method of using a CALR mutation as a prognostic marker for measuring response to treatment with an SAP protein of the disclosure. In one embodiment, the method comprises: (i) measuring a first mutant allele burden of a mutation in CALR, wherein said first mutant allele burden is measured before administration of the SAP protein; (ii) measuring a second mutant allele burden of the same mutation measured in (i), wherein said second mutant allele burden is measured after administration of the SAP protein; and (iii) measuring the difference between the second mutant allele burden and the first mutant allele burden. In a further embodiment, a decrease in the second mutant allele burden relative to the first mutant allele burden indicates a positive prognosis. In an alternative embodiment, if there is no change in the second mutant allele burden relative to the first mutant allele burden indicates a neutral or negative prognosis. In some embodiments, the CALR mutation is a missense mutation, a deletion mutation, an insertion mutation or a translocation. In some embodiments, mutations in CALR include insertion/deletion mutations in exon 9 of CALR. The SAP proteins of the disclosure (such as a recombinant human SAP protein, such as a glycosylated SAP protein) are used, alone or in combination with an additional agent, to treat a myeloproliferative disorder. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In certain embodiments, any of the foregoing methods further comprise assaying for one or more mutations in one or more MPD-associated genes such as but not limited to, JAK2, MPL, ASXL1, EZH2, SRSF2, IDH1, or IDH2. In some embodiments, any of the foregoing methods further comprise assaying for one or more mutations in TET2, CBL, IKZF1, LNK, DNMT3A, CUX1, U2AF1, or SF3B1. In some embodiments, any of the foregoing methods further comprise assaying for one or more cytogenetic abnormalities such as monosomal karyotype, inv(3), i(17q), −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities. In some embodiments of any of the above aspects of the disclosure, the MPD is polycythemia vera, essential thrombocythemia, myelofibrosis, or an unclassified myeloproliferative disease. In some embodiments, the myelofibrosis is primary myelofibrosis, post-PV myelofibrosis, or post-ET myelofibrosis.

Methods of the disclosure involve evaluating a sample containing nucleic acids from an individual having or suspected of having an MPD for the presence or absence of CALR mutations. The sample may be any suitable biological sample including, for example, whole blood (i.e., CALR nucleic acid being extracted from the cellular fraction), plasma, serum, bone marrow, and tissue samples (e.g., biopsy and paraffin-embedded tissue). The CALR nucleic acid may be any convenient nucleic acid type including, for example, genomic DNA, RNA (e.g., mRNA), or cDNA prepared from subject RNA. Alternatively, the CALR nucleic acid mutation may be inferred by assessing the CALR protein from the individual. For example, identification of a mutant CALR protein is indicative of a mutation in the CALR gene. Suitable detection methodologies include oligonucleotide probe hybridization, primer extension reaction, nucleic acid sequencing, and protein sequencing. In some embodiments, the individual is screened for the presence of other pathological mutations in one or more additional MPD-associated genes (e.g., JAK2, MPL, ASXL1, EZH2, SRSF2, IDH1, or IDH2) either simultaneously or prior to screening for the CALR nucleic acid mutation. In some embodiments, one or more (e.g., 2, 3, 4, 5, 6, 7, or 8) mutations in addition to a CALR mutation are used as a prognostic marker for measuring response to treatment with an SAP protein of the disclosure.

### ASXL1

Additional sex combs like transcriptional regulator 1 (ASXL1) is needed for normal hematopoiesis and is thought to be involved in activation of transcription factors and transcriptional repression. ASXL1 mutations are thought to contribute to epigenetic dysregulation of effects in myeloproliferative neoplasms. ASXL1 mutations involve exon 12 and truncate the pleckstrin homology domain of ASXL1. In a recent study, ASXL1 mutational frequencies were 13% in PMF, 23% in post-PV/ET MF, and 18% in blast-phase MPN (The same study demonstrated co-occurrence of mutant ASXL1 with TET2. JAK2, EZH2, IDH and MPL mutations (Abdel-Wahab et al. ASH Annu Meet Abstr. 2010, 116:3070).

ASXL1 genomic nucleic acid is located in human chromosome 20. An exemplary sequence of all or portions of human ASXL1 mRNA includes but is not limited to GenBank Accession number NM_001164603 (SEQ ID NO: 11). These sequences are incorporated herein by reference.

For the ASXL1 nucleic acid sequence, a “mutation” means a ASXL1 nucleic acid sequence that includes at least one nucleic acid variation as compared to reference sequence GenBank accession number NM_001164603 (SEQ ID NO: 11). A mutation in ASXL1 nucleic acid may result in a change in the encoded polypeptide sequence or the mutation may be silent with respect to the encoded polypeptide sequence. A change in an amino acid sequence may be determined as compared to NP_001158075 (SEQ ID NO: 12) as a reference amino acid sequence.

In some embodiments, the ASXL1 mutation is a missense mutation, a deletion mutation, an insertion mutation or a translocation. In some embodiments, mutations in ASXL1 include insertion/deletion mutations in exon 12 of ASXL1.

In some embodiments, the disclosure provides a method of treating an MPD comprising administering a therapeutically effective amount of an SAP protein to a subject carrying a mutation in ASXL1 (e.g., some of the hematopoietic cells of the subject carry an ASXL1 mutation). In some embodiments, the disclosure provides a method of treating an MPD comprising administering a therapeutically effective amount of an SAP protein to a subject carrying a mutation (e.g., the mutation may be found in some hematopoietic cells of the subject) in ASXL1, according to a dosage regimen effective to reduce mutant ASXL1 allele burden in said subject. In some embodiments, the disclosure provides a method for reducing mutant allele burden in ASXL1 in a subject suffering from a myeloproliferative disorder. In some embodiments, the disclosure provides methods of monitoring the effectiveness of an SAP protein therapy for an MPD based on ASXL1 mutational status. In one embodiment, the method comprises: (i) measuring a first mutant allele burden of a mutation in ASXL1, wherein said first mutant allele burden is measured before administration of the SAP protein; (ii) measuring a second mutant allele burden of the same mutation measured in (i), wherein said second mutant allele burden is measured after administration of the SAP protein; and (iii) identifying a difference between the second mutant allele burden and the first mutant allele burden. In a further embodiment, a decrease in the second mutant allele burden relative to the first mutant allele burden indicates that the administration of the SAP protein is effective in treating the myeloproliferative disorder and the dosage regimen may be maintained or modified to decrease the dosage and/or frequency of administration. In an alternative embodiment, if there is no change in the second mutant allele burden relative to the first mutant allele burden, the dosage regimen may be modified to increase the dosage and/or frequency of administration. In one aspect, the disclosure provides methods of determining responsiveness to SAP protein therapy based on the presence or absence of one or more somatic mutations in ASXL1. In some embodiments, the method comprises (i) determining whether the cells of a subject having a myeloproliferative disorder carry a mutation associated with the myeloproliferative disorder in ASXL1; and if the subject carries said mutant allele (ii) administering a therapeutically effective amount of an SAP protein to the subject. In one aspect, the disclosure provides a method of using an ASXL1 mutation as a prognostic marker for measuring response to treatment with an SAP protein of the disclosure. In one embodiment, the method comprises: (i) measuring a first mutant allele burden of a mutation in ASXL1, wherein said first mutant allele burden is measured before administration of the SAP protein; (ii) measuring a second mutant allele burden of the same mutation measured in (i), wherein said second mutant allele burden is measured after administration of the SAP protein; and (iii) measuring the difference between the second mutant allele burden and the first mutant allele burden. In a further embodiment, a decrease in the second mutant allele burden relative to the first mutant allele burden indicates a positive prognosis. In an alternative embodiment, if there is no change in the second mutant allele burden relative to the first mutant allele burden indicates a neutral or negative prognosis. In some embodiments, the ASXL1 mutation is a missense mutation, a deletion mutation, an insertion mutation or a translocation. In some embodiments, mutations in ASXL1 include insertion/deletion mutations in exon 12 of ASXL1. The SAP proteins of the disclosure (such as a recombinant human SAP protein, such as a glycosylated SAP protein) are used, alone or in combination with an additional agent, to treat a myeloproliferative disorder. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In certain embodiments, any of the foregoing methods further comprise assaying for one or more mutations in one or more MPD-associated genes such as but not limited to, JAK2, MPL, CALR, EZH2, SRSF2, IDH1, or IDH2. In some embodiments, any of the foregoing methods further comprise assaying for one or more mutations in TET2, CBL, IKZF1, LNK, DNMT3A, CUX1, U2AF1, or SF3B1. In some embodiments, any of the foregoing methods further comprise assaying for one or more cytogenetic abnormalities such as monosomal karyotype, inv(3), i(17q), −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities. In some embodiments of any of the above aspects of the disclosure, the MPD is polycythemia vera, essential thrombocythemia, myelofibrosis, or an unclassified myeloproliferative disease. In some embodiments, the myelofibrosis is primary myleofibrosis, post-PV myelofibrosis, or post-ET myelofibrosis.

Methods of the disclosure involve evaluating a sample containing nucleic acids form an individual having or suspected of having an MPD for the presence or absence of ASXL1 mutations. The sample may be any suitable biological sample including, for example, whole blood (i.e., ASXL1 nucleic acid being extracted from the cellular fraction), plasma, serum, bone marrow, and tissue samples (e.g., biopsy and paraffin-embedded tissue). The ASXL1 nucleic acid may be any convenient nucleic acid type including, for example, genomic DNA, RNA (e.g., mRNA), or cDNA prepared from subject RNA. Alternatively, the ASXL1 nucleic acid mutation may be inferred by assessing the ASXL1 protein from the individual. For example, identification of a mutant ASXL1 protein is indicative of a mutation in the ASXL1 gene. Suitable detection methodologies include oligonucleotide probe hybridization, primer extension reaction, nucleic acid sequencing, and protein sequencing. In some embodiments, the individual is screened for the presence of other pathological mutations in one or more additional MPD-associated genes (e.g., JAK2, MPL, CALR, EZH2, SRSF2, IDH1, or IDH2) either simultaneously or prior to screening for the ASXL1 nucleic acid mutation. In some embodiments, one or more (e.g., 2, 3, 4, 5, 6, 7, or 8) mutations in addition to an ASXL11 mutation are used as a prognostic marker for measuring response to treatment with an SAP protein of the disclosure.

### EZH2

Enhancer of zeste 2 polycomb repressive complex 2 subunit (EZH2) is part of a methyltransferase (polycomb-repressive complex 2 associated with H3 Lys-27 trimethylation). In one study, mutational frequencies of EZH2 were 13% in CMML, 13% in atypical CML, 13% in MF (PMF or post-PV/ET MF), 10% in MDS/MPN-U, 6% in MDS, 3% in PV and 3% in hypereosinophilic syndrome/chronic eosinophilic leukemia (Ernst et al. Nat Genet. 2010, 42:722-726). EZH2 variants in this study included missense, frameshift or stop mutations expected to result in premature chain termination or truncation of critical domains. It is thought that the MPN-associated EZH2 mutations have a tumor suppressor activity.

EZH2 genomic nucleic acid is located in human chromosome 7. An exemplary sequence of all or portions of human EZH2 mRNA includes but is not limited to GenBank Accession number NM_001203247 (SEQ ID NO: 13). These sequences are incorporated herein by reference.

For the EZH2 nucleic acid sequence, a “mutation” means a EZH2 nucleic acid sequence that includes at least one nucleic acid variation as compared to reference sequence GenBank accession number NM_001203247 (SEQ ID NO: 13). A mutation in EZH2 nucleic acid may result in a change in the encoded polypeptide sequence or the mutation may be silent with respect to the encoded polypeptide sequence. A change in an amino acid sequence may be determined as compared to NP_001190176 (SEQ ID NO: 14) as a reference amino acid sequence.

In some embodiments, the EZH2 mutation is a missense mutation, a deletion mutation, an insertion mutation or a translocation.

In some embodiments, the disclosure provides a method of treating an MPD comprising administering a therapeutically effective amount of an SAP protein to a subject carrying a mutation in EZH2 (e.g., some of the hematopoietic cells of the subject carry an EZH2 mutation). In some embodiments, the disclosure provides a method of treating an MPD comprising administering a therapeutically effective amount of an SAP protein to a subject carrying a mutation (e.g., the mutation may be found in some hematopoietic cells of the subject) in EZH2, according to a dosage regimen effective to reduce mutant EZH2 allele burden in said subject. In some embodiments, the disclosure provides a method for reducing mutant allele burden in EZH2 in a subject suffering from a myeloproliferative disorder. In some embodiments, the disclosure provides methods of monitoring the effectiveness of an SAP protein therapy for an MPD based on EZH2 mutational status. In one embodiment, the method comprises: (i) measuring a first mutant allele burden of a mutation in EZH2, wherein said first mutant allele burden is measured before administration of the SAP protein; (ii) measuring a second mutant allele burden of the same mutation measured in (i), wherein said second mutant allele burden is measured after administration of the SAP protein; and (iii) identifying a difference between the second mutant allele burden and the first mutant allele burden. In a further embodiment, a decrease in the second mutant allele burden relative to the first mutant allele burden indicates that the administration of the SAP protein (e.g., an SAP protein) is effective in treating the myeloproliferative disorder and the dosage regimen may be maintained or modified to decrease the dosage and/or frequency of administration. In an alternative embodiment, if there is no change in the second mutant allele burden relative to the first mutant allele burden, the dosage regimen may be modified to increase the dosage and/or frequency of administration. In one aspect, the disclosure provides methods of determining responsiveness to SAP protein therapy based on the presence or absence of one or more somatic mutations in EZH2. In some embodiments, the method comprises (i) determining whether the cells of a subject having a myeloproliferative disorder carry a mutation associated with the myeloproliferative disorder in EZH2; and if the subject carries said mutant allele (ii) administering a therapeutically effective amount of an SAP protein to the subject. In one aspect, the disclosure provides a method of using an EZH2 mutation as a prognostic marker for measuring response to treatment with an SAP protein of the disclosure. In one embodiment, the method comprises: (i) measuring a first mutant allele burden of a mutation in EZH2, wherein said first mutant allele burden is measured before administration of the SAP protein; (ii) measuring a second mutant allele burden of the same mutation measured in (i), wherein said second mutant allele burden is measured after administration of the SAP protein; and (iii) measuring the difference between the second mutant allele burden and the first mutant allele burden. In a further embodiment, a decrease in the second mutant allele burden relative to the first mutant allele burden indicates a positive prognosis. In an alternative embodiment, if there is no change in the second mutant allele burden relative to the first mutant allele burden indicates a neutral or negative prognosis. In some embodiments, the EZH2 mutation is a missense mutation, a deletion mutation, an insertion mutation or a translocation. The SAP proteins of the disclosure (such as a recombinant human SAP protein, such as a glycosylated SAP protein) are used, alone or in combination with an additional agent, to treat a myeloproliferative disorder. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In certain embodiments, any of the foregoing methods further comprise assaying for one or more mutations in one or more MPD-associated genes such as but not limited to, JAK2, MPL, CALR, ASXL1, SRSF2, IDH, or IDH2. In some embodiments, any of the foregoing methods further comprise assaying for one or more mutations in TET2, CBL, IKZF1, LNK, DNMT3A, CUX1, U2AF1, or SF3B1. In some embodiments, any of the foregoing methods further comprise assaying for one or more cytogenetic abnormalities such as monosomal karyotype, inv(3), i(17q), −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities. In some embodiments of any of the above aspects of the disclosure, the MPD is polycythemia vera, essential thrombocythemia, myelofibrosis, or an unclassified myeloproliferative disease. In some embodiments, the myelofibrosis is primary myleofibrosis, post-PV myelofibrosis, or post-ET myelofibrosis.

Methods of the disclosure involve evaluating a sample containing nucleic acids from an individual having or suspected of having an MPD for the presence or absence of EZH2 mutations. The sample may be any suitable biological sample including, for example, whole blood (i.e., EZH2 nucleic acid being extracted from the cellular fraction), plasma, serum, bone marrow, and tissue samples (e.g., biopsy and paraffin-embedded tissue). The EZH2 nucleic acid may be any convenient nucleic acid type including, for example, genomic DNA, RNA (e.g., mRNA), or cDNA prepared from subject RNA. Alternatively, the EZH2 nucleic acid mutation may be inferred by assessing the EZH2 protein from the individual. For example, identification of a mutant EZH2 protein is indicative of a mutation in the EZH2 gene. Suitable detection methodologies include oligonucleotide probe hybridization, primer extension reaction, nucleic acid sequencing, and protein sequencing. In some embodiments, the individual is screened for the presence of other pathological mutations in one or more additional MPD-associated genes (e.g., JAK2, MPL, CALR, ASXL1, SRSF2, IDH1, or IDH2) either simultaneously or prior to screening for the EZH2 nucleic acid mutation. In some embodiments, one or more (e.g., 2, 3, 4, 5, 6, 7, or 8) mutations in addition to an EZH2 mutation are used as a prognostic marker for measuring response to treatment with an SAP protein of the disclosure.

Serine/arginine-rich splicing factor 2 (SRSF2) is a component of the RNA splicing machinery. SRSF2 mutations alter pre-mRNA splicing, are relatively common in primary myelofibrosis, and appear to be predictive of poor outcome. In one study, 187 PMF patients were studied and it was found that 17% harbored SRSF2 mutations, including missense mutations such as P95H, P95L, P95R, and P95S, a 24-bp deletion (delP95-R102) and an insertion mutation 274-275insACC (G93D; P95R). SRSF2 mutations clustered with IDH mutations (Lasho et al. Blood 2012, 120(20):4168-71).

SRSF2 genomic nucleic acid is located in human chromosome 17. An exemplary sequence of all or portions of human SRSF2 mRNA includes but is not limited to GenBank Accession number NM_001195427 (SEQ ID NO: 15). These sequences are incorporated herein by reference.

For the SRSF2 nucleic acid sequence, a “mutation” means a SRSF2 nucleic acid sequence that includes at least one nucleic acid variation as compared to reference sequence GenBank accession number NM_001195427 (SEQ ID NO: 15). A mutation in SRSF2 nucleic acid may result in a change in the encoded polypeptide sequence or the mutation may be silent with respect to the encoded polypeptide sequence. A change in an amino acid sequence may be determined as compared to NP_001182356 (SEQ ID NO: 16) as a reference amino acid sequence.

In some embodiments, the SRSF2 mutation is a missense mutation, a deletion mutation, an insertion mutation or a translocation. In some embodiments, mutations in SRSF2 include P95H. P95L. P95R, P95S, delP95-R102 or G93D; P95R insertion.

In some embodiments, the disclosure provides a method of treating an MPD comprising administering a therapeutically effective amount of an SAP protein to a subject carrying a mutation in SRSF2 (e.g., some of the hematopoietic cells of the subject carry an SRSF2 mutation). In some embodiments, the disclosure provides a method of treating an MPD comprising administering a therapeutically effective amount of an SAP protein to a subject carrying a mutation (e.g., the mutation may be found in some hematopoietic cells of the subject) in SRSF2, according to a dosage regimen effective to reduce mutant SRSF2 allele burden in said subject. In some embodiments, the disclosure provides a method for reducing mutant allele burden in SRSF2 in a subject suffering from a myeloproliferative disorder. In some embodiments, the disclosure provides methods of monitoring the effectiveness of an SAP protein therapy for an MPD based on SRSF2 mutational status. In one embodiment, the method comprises: (i) measuring a first mutant allele burden of a mutation in SRSF2, wherein said first mutant allele burden is measured before administration of the SAP protein; (ii) measuring a second mutant allele burden of the same mutation measured in (i), wherein said second mutant allele burden is measured after administration of the SAP protein; and (iii) identifying a difference between the second mutant allele burden and the first mutant allele burden. In a further embodiment, a decrease in the second mutant allele burden relative to the first mutant allele burden indicates that the administration of the SAP protein is effective in treating the myeloproliferative disorder and the dosage regimen may be maintained or modified to decrease the dosage and/or frequency of administration. In an alternative embodiment, if there is no change in the second mutant allele burden relative to the first mutant allele burden, the dosage regimen may be modified to increase the dosage and/or frequency of administration. In one aspect, the disclosure provides methods of determining responsiveness to SAP protein therapy based on the presence or absence of one or more somatic mutations in SRSF2. In some embodiments, the method comprises (i) determining whether the cells of a subject having a myeloproliferative disorder carry a mutation associated with the myeloproliferative disorder in SRSF2; and if the subject carries said mutant allele (ii) administering a therapeutically effective amount of an SAP protein to the subject. In one aspect, the disclosure provides a method of using an SRSF2 mutation as a prognostic marker for measuring response to treatment with an SAP protein of the disclosure. In one embodiment, the method comprises: (i) measuring a first mutant allele burden of a mutation in SRSF2, wherein said first mutant allele burden is measured before administration of the SAP protein; (ii) measuring a second mutant allele burden of the same mutation measured in (i), wherein said second mutant allele burden is measured after administration of the SAP protein; and (iii) measuring the difference between the second mutant allele burden and the first mutant allele burden. In a further embodiment, a decrease in the second mutant allele burden relative to the first mutant allele burden indicates a positive prognosis. In an alternative embodiment, if there is no change in the second mutant allele burden relative to the first mutant allele burden indicates a neutral or negative prognosis. In some embodiments, the SRSF2 mutation is a missense mutation, a deletion mutation, an insertion mutation or a translocation. In some embodiments, mutations in SRSF2 include P95H, P95L. P95R, P95S, delP95-R102 or G93D; P95R insertion. The SAP proteins of the disclosure (such as a recombinant human SAP protein, such as a glycosylated SAP protein) are used, alone or in combination with an additional agent, to treat a mycloproliferative disorder. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In certain embodiments, any of the foregoing methods further comprise assaying for one or more mutations in one or more MPD-associated genes such as but not limited to, JAK2, MPL, CALR, ASXL1, EZH2, IDH1, or IDH2. In some embodiments, any of the foregoing methods further comprise assaying for one or more mutations in TET2, CBL, IKZF1, LNK, DNMT3A, CUX1, U2AF1, or SF3B1. In some embodiments, any of the foregoing methods further comprise assaying for one or more cytogenetic abnormalities such as monosomal karyotype, inv(3), i(17q), −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities. In some embodiments of any of the above aspects of the disclosure, the MPD is polycythemia vera, essential thrombocythemia, myelofibrosis, or an unclassified myeloproliferative disease. In some embodiments, the myelofibrosis is primary myleofibrosis, post-PV myelofibrosis, or post-ET myelofibrosis.

Methods of the disclosure involve evaluating a sample containing nucleic acids from an individual having or suspected of having an MPD for the presence or absence of SRSF2 mutations. The sample may be any suitable biological sample including, for example, whole blood (i.e., SRSF2 nucleic acid being extracted from the cellular fraction), plasma, serum, bone marrow, and tissue samples (e.g., biopsy and paraffin-embedded tissue). The SRSF2 nucleic acid may be any convenient nucleic acid type including, for example, genomic DNA, RNA (e.g., mRNA), or cDNA prepared from subject RNA. Alternatively, the SRSF2 nucleic acid mutation may be inferred by assessing the SRSF2 protein from the individual. For example, identification of a mutant SRSF2 protein is indicative of a mutation in the SRSF2 gene. Suitable detection methodologies include oligonucleotide probe hybridization, primer extension reaction, nucleic acid sequencing, and protein sequencing. In some embodiments, the individual is screened for the presence of other pathological mutations in one or more additional MPD-associated genes (e.g., JAK2, MPL, CALR, ASXL1, EZH2, IDH1, or IDH2) either simultaneously or prior to screening for the SRSF2 nucleic acid mutation. In some embodiments, one or more (e.g., 2, 3, 4, 5, 6, 7, or 8) mutations in addition to an SRSF2 mutation are used as a prognostic marker for measuring response to treatment with an SAP protein of the disclosure.

### IDH1 and IDH2

Isocitrate dehydrogenase (IDH) mutations involve exon 4 and affect three arginine residues, R132 and R172 in IDH1 and R140 in IDH2, IDH1 mutations result in loss of isocitrate to 2-ketoglutarate conversion activity and a gain of 2-ketoglutarate to 2-hydroxyglutarate conversion activity. One study identified IDH mutational frequencies of ˜2% in PV, 1% in ET, 4% in PMF and 22% in blast-phase MPN (Tefferi et al. Leukemia. 2010, 24:1302-1309). IDH-mutated patients were more likely to be nullizygous for JAK2 46/1 haplotype and less likely to display complex karyotype.

IDH1 genomic nucleic acid is located in human chromosome 2. An exemplary sequence of all or portions of human IDH1 mRNA includes but is not limited to GenBank Accession number NM_005896 (SEQ ID NO: 17). These sequences are incorporated herein by reference.

For the IDH1 nucleic acid sequence, a “mutation” means a IDH1 nucleic acid sequence that includes at least one nucleic acid variation as compared to reference sequence GenBank accession number NM_005896 (SEQ ID NO: 17). A mutation in IDH1 nucleic acid may result in a change in the encoded polypeptide sequence or the mutation may be silent with respect to the encoded polypeptide sequence. A change in an amino acid sequence may be determined as compared to NP_005887 (SEQ ID NO: 18) as a reference amino acid sequence.

In some embodiments, the IDH1 mutation is a missense mutation, a deletion mutation, an insertion mutation or a translocation. In some embodiments, mutations in IDH1 include insertion/deletion mutations in exon 4 of IDH1. In some embodiments, mutations in IDH1 include missense mutations at R132.

In some embodiments, the disclosure provides a method of treating an MPD comprising administering a therapeutically effective amount of an SAP protein to a subject carrying a mutation in IDH1 (e.g., some of the hematopoietic cells of the subject carry an IDH1 mutation). In some embodiments, the disclosure provides a method of treating an MPD comprising administering a therapeutically effective amount of an SAP protein to a subject carrying a mutation (e.g., the mutation may be found in some hematopoietic cells of the subject) in IDH1, according to a dosage regimen effective to reduce mutant IDH1 allele burden in said subject. In some embodiments, the disclosure provides a method for reducing mutant allele burden in IDH1 in a subject suffering from a myeloproliferative disorder. In some embodiments, the disclosure provides methods of monitoring the effectiveness of an SAP protein therapy for an MPD based on IDH1 mutational status. In one embodiment, the method comprises: (i) measuring a first mutant allele burden of a mutation in IDH1, wherein said first mutant allele burden is measured before administration of the SAP protein; (ii) measuring a second mutant allele burden of the same mutation measured in (i), wherein said second mutant allele burden is measured after administration of the SAP protein; and (iii) identifying a difference between the second mutant allele burden and the first mutant allele burden. In a further embodiment, a decrease in the second mutant allele burden relative to the first mutant allele burden indicates that the administration of the SAP protein is effective in treating the myeloproliferative disorder and the dosage regimen may be maintained or modified to decrease the dosage and/or frequency of administration. In an alternative embodiment, if there is no change in the second mutant allele burden relative to the first mutant allele burden, the dosage regimen may be modified to increase the dosage and/or frequency of administration. In one aspect, the disclosure provides methods of determining responsiveness to SAP protein or agonist therapy based on the presence or absence of one or more somatic mutations in IDH1. In some embodiments, the method comprises (i) determining whether the cells of a subject having a myeloproliferative disorder carry a mutation associated with the myeloproliferative disorder in IDH1; and if the subject carries said mutant allele (ii) administering a therapeutically effective amount of an SAP protein to the subject. In one aspect, the disclosure provides a method of using an IDH1 mutation as a prognostic marker for measuring response to treatment with an SAP protein of the disclosure. In one embodiment, the method comprises: (i) measuring a first mutant allele burden of a mutation in IDH1, wherein said first mutant allele burden is measured before administration of the SAP protein; (ii) measuring a second mutant allele burden of the same mutation measured in (i), wherein said second mutant allele burden is measured after administration of the SAP protein; and (iii) measuring the difference between the second mutant allele burden and the first mutant allele burden. In a further embodiment, a decrease in the second mutant allele burden relative to the first mutant allele burden indicates a positive prognosis. In an alternative embodiment, if there is no change in the second mutant allele burden relative to the first mutant allele burden indicates a neutral or negative prognosis. In some embodiments, the IDH1 mutation is a missense mutation, a deletion mutation, an insertion mutation or a translocation. In some embodiments, mutations in IDH1 include insertion/deletion mutations in exon 4 of IDH1. In some embodiments, mutations in IDH1 include missense mutations at R132. The SAP proteins of the disclosure (such as a recombinant human SAP protein, such as a glycosylated SAP protein) are used, alone or in combination with an additional agent, to treat a myeloproliferative disorder. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In certain embodiments, any of the foregoing methods further comprise assaying for one or more mutations in one or more MPD-associated genes such as but not limited to, JAK2, MPL, CALR, ASXL1, EZH2, SRSF2, or IDH2. In some embodiments, any of the foregoing methods further comprise assaying for one or more mutations in TET2, CBL, IKZF1, LNK, DNMT3A, CUX1, U2AF1, or SF3B1. In some embodiments, any of the foregoing methods further comprise assaying for one or more cytogenetic abnormalities such as monosomal karyotype, inv(3), i(17q), −7/7q-, 11q or 12p abnormalities, complex non-monosomal, 5q-, +8, other autosomal trisomies except +9, sole abnormalities of 20q-, 1 q duplication or any other translocation, and −Y or other sex chromosome abnormality, normal or sole abnormalities of 13q- or +9, or other sole abnormalities. In some embodiments of any of the above aspects of the disclosure, the MPD is polycythemia vera, essential thrombocythemia, myelofibrosis, or an unclassified myeloproliferative disease. In some embodiments, the myelofibrosis is primary myleofibrosis, post-PV myelofibrosis, or post-ET myelofibrosis.

Methods of the disclosure involve evaluating a sample containing nucleic acids from an individual having or suspected of having an MPD for the presence or absence of IDH1 mutations. The sample may be any suitable biological sample including, for example, whole blood (i.e., IDH1 nucleic acid being extracted from the cellular fraction), plasma, serum, bone marrow, and tissue samples (e.g., biopsy and paraffin-embedded tissue). The IDH1 nucleic acid may be any convenient nucleic acid type including, for example, genomic DNA, RNA (e.g., mRNA), or cDNA prepared from subject RNA. Alternatively, the IDH1 nucleic acid mutation may be inferred by assessing the IDH1 protein from the individual. For example, identification of a mutant IDH1 protein is indicative of a mutation in the IDH1 gene. Suitable detection methodologies include oligonucleotide probe hybridization, primer extension reaction, nucleic acid sequencing, and protein sequencing. In some embodiments, the individual is screened for the presence of other pathological mutations in one or more additional MPD-associated genes (e.g., JAK2, MPL, CALR, ASXL1, EZH2, SRSF2, or IDH2) either simultaneously or prior to screening for the IDH1 nucleic acid mutation. In some embodiments, one or more (e.g., 2, 3, 4, 5, 6, 7, or 8) mutations in addition to an IDH1 mutation are used as a prognostic marker for measuring response to treatment with an SAP protein of the disclosure.

IDH2 genomic nucleic acid is located in human chromosome 15. An exemplary sequence of all or portions of human IDH1 mRNA includes but is not limited to GenBank Accession number NM_001289910 (SEQ ID NO: 19). These sequences are incorporated herein by reference.

For the IDH2 nucleic acid sequence, a “mutation” means a IDH2 nucleic acid sequence that includes at least one nucleic acid variation as compared to reference sequence GenBank accession number NM_001289910 (SEQ ID NO: 19). A mutation in IDH2 nucleic acid may result in a change in the encoded polypeptide sequence or the mutation may be silent with respect to the encoded polypeptide sequence. A change in an amino acid sequence may be determined as compared to NP_001276839 (SEQ ID NO: 20) as a reference amino acid sequence.

In some embodiments, the IDH2 mutation is a missense mutation, a deletion mutation, an insertion mutation or a translocation. In some embodiments, mutations in IDH2 include insertion/deletion mutations in exon 4 of IDH2. In some embodiments, mutations in IDH2 include missense mutations at R172 or R140.

In some embodiments, the disclosure provides a method of treating an MPD comprising administering a therapeutically effective amount of an SAP protein to a subject carrying a mutation in IDH2 (e.g., some of the hematopoietic cells of the subject carry an IDH2 mutation). In some embodiments, the disclosure provides a method of treating an MPD comprising administering a therapeutically effective amount of an SAP protein to a subject carrying a mutation (e.g., the mutation may be found in some hematopoietic cells of the subject) in IDH2, according to a dosage regimen effective to reduce mutant IDH2 allele burden in said subject. In some embodiments, the disclosure provides a method for reducing mutant allele burden in IDH2 in a subject suffering from a myeloproliferative disorder. In some embodiments, the disclosure provides methods of monitoring the effectiveness of an SAP protein therapy for an MPD based on IDH2 mutational status. In one embodiment, the method comprises: (i) measuring a first mutant allele burden of a mutation in IDH2, wherein said first mutant allele burden is measured before administration of the SAP protein; (ii) measuring a second mutant allele burden of the same mutation measured in (i), wherein said second mutant allele burden is measured after administration of the SAP protein; and (iii) identifying a difference between the second mutant allele burden and the first mutant allele burden. In a further embodiment, a decrease in the second mutant allele burden relative to the first mutant allele burden indicates that the administration of the SAP protein is effective in treating the myeloproliferative disorder and the dosage regimen may be maintained or modified to decrease the dosage and/or frequency of administration. In an alternative embodiment, if there is no change in the second mutant allele burden relative to the first mutant allele burden, the dosage regimen may be modified to increase the dosage and/or frequency of administration. In one aspect, the disclosure provides methods of determining responsiveness to SAP protein therapy based on the presence or absence of one or more somatic mutations in IDH2. In some embodiments, the method comprises (i) determining whether the cells of a subject having a myeloproliferative disorder carry a mutation associated with the myeloproliferative disorder in IDH2; and if the subject carries said mutant allele (ii) administering a therapeutically effective amount of an SAP protein to the subject. In one aspect, the disclosure provides a method of using an IDH2 mutation as a prognostic marker for measuring response to treatment with an SAP protein of the disclosure. In one embodiment, the method comprises: (i) measuring a first mutant allele burden of a mutation in IDH2, wherein said first mutant allele burden is measured before administration of the SAP protein; (ii) measuring a second mutant allele burden of the same mutation measured in (i), wherein said second mutant allele burden is measured after administration of the SAP protein; and (iii) measuring the difference between the second mutant allele burden and the first mutant allele burden. In a further embodiment, a decrease in the second mutant allele burden relative to the first mutant allele burden indicates a positive prognosis. In an alternative embodiment, if there is no change in the second mutant allele burden relative to the first mutant allele burden indicates a neutral or negative prognosis. In some embodiments, the IDH2 mutation is a missense mutation, a deletion mutation, an insertion mutation or a translocation. In some embodiments, mutations in IDH2 include insertion/deletion mutations in exon 4 of IDH2. In some embodiments, mutations in IDH2 include missense mutations at R172 or R140. The SAP proteins of the disclosure (such as a recombinant human SAP protein, such as a glycosylated SAP protein) are used, alone or in combination with an additional agent, to treat a myeloproliferative disorder. In some embodiments, an SAP protein of the disclosure (such as a recombinant human SAP protein, such as a glycosylated SAP protein) is used as a monotherapy. In some embodiments, an SAP protein of the disclosure (such as a recombinant human SAP protein, such as a glycosylated SAP protein) is used in combination with an anti-cancer agent. In certain embodiments, any of the foregoing methods further comprise assaying for one or more mutations in one or more MPD-associated genes such as but not limited to, JAK2, MPL, CALR, ASXL1, EZH2, SRSF2, or IDH1. In some embodiments, any of the foregoing methods further comprise assaying for one or more mutations in TET2, CBL, IKZF1, LNK DNMT3A, CUX1, U2AF1, or SF3B1. In some embodiments, any of the foregoing methods further comprise assaying for one or more cytogenetic abnormalities such as monosomal karyotype, inv(3), i(17q), −7/7q-, 11q or 12p abnormalities, complex non-monosomal, 5q-, +8, other autosomal trisomies except +9, sole abnormalities of 20q-, 1q duplication or any other translocation, and −Y or other sex chromosome abnormality, normal or sole abnormalities of 13q- or +9, or other sole abnormalities. In some embodiments of any of the above aspects of the disclosure, the MPD is polycythemia vera, essential thrombocythemia, myelofibrosis, or an unclassified myeloproliferative disease. In some embodiments, the myelofibrosis is primary myleofibrosis, post-PV myelofibrosis, or post-ET myelofibrosis.

Methods of the disclosure involve evaluating a sample containing nucleic acids from an individual having or suspected of having an MPD for the presence or absence of IDH2 mutations. The sample may be any suitable biological sample including, for example, whole blood (i.e., IDH2 nucleic acid being extracted from the cellular fraction), plasma, serum, bone marrow, and tissue samples (e.g., biopsy and paraffin-embedded tissue). The IDH2 nucleic acid may be any convenient nucleic acid type including, for example, genomic DNA, RNA (e.g., mRNA), or cDNA prepared from subject RNA. Alternatively, the IDH2 nucleic acid mutation may be inferred by assessing the IDH2 protein from the individual. For example, identification of a mutant IDH2 protein is indicative of a mutation in the IDH2 gene. Suitable detection methodologies include oligonucleotide probe hybridization, primer extension reaction, nucleic acid sequencing, and protein sequencing. In some embodiments, the individual is screened for the presence of other pathological mutations in one or more additional MPD-associated genes (e.g., JAK2, MPL, CALR, ASXL1, EZH2, SRSF2, or IDH1) either simultaneously or prior to screening for the IDH2 nucleic acid mutation. In some embodiments, one or more (e.g., 2, 3, 4, 5, 6, 7, or 8) mutations in addition to an IDH2 mutation are used as a prognostic marker for measuring response to treatment with an SAP protein of the disclosure.

In certain methods of any of the foregoing, the disclosure provides methods of treating a subject determined to comprise a mutation associated with an MPD, such as a mutation in one or more of the foregoing genes (e.g., some of the subject's cells carry the mutation). In certain embodiments of any of the foregoing, the subject is heterozygous or homozygous. In certain embodiments, the subject carries more than one mutation associated with an MPD (e.g., 2, 3 or more than 3).

### Detection Methods

The methods of the disclosure can also be used to detect mutations in a myeloproliferative disorder-associated gene (e.g. JAK2, MPL, CALR, ASXL1, EZH2, SRSF2, IDH1, or IDH2) or to detect other genetic alterations such as cytogenetic abnormalities. In certain embodiments, the methods include detecting, in a sample of cells (e.g., bodily fluid cells such as blood cells, bone marrow cells, etc.) from the subject, the presence or absence of a genetic mutation in a myeloproliferative disorder-associated gene. For example, such genetic mutations can be detected by ascertaining the existence of at least one of: 1) a deletion of one or more nucleotides from one or more genes; 2) an addition of one or more nucleotides to one or more genes; 3) a substitution of one or more nucleotides of one or more genes, 4) a chromosomal rearrangement (e.g., translocation) of one or more genes; 5) an alteration in the level of a messenger RNA transcript of one or more genes; 6) aberrant modification of one or more genes, such as of the methylation pattern of the genomic DNA; 7) the presence of a non-wild type splicing pattern of a messenger RNA transcript of one or more genes; 8) a non-wild type level of a one or more proteins; 9) allelic loss of one or more genes; and 10) inappropriate post-translational modification of one or more proteins. As described herein, there are a large number of assays known in the art which can be used for detecting mutations in one or more genes. In some embodiments, the mutational status of gene is measured by collecting peripheral blood samples, extracting DNA from the samples, and analyzing by PCR. In some embodiments, genomic DNA is extracted from peripheral blood leukocytes. In some embodiments, genomic DNA is extracted from peripheral blood granulocytes. In some embodiments, the mutations in one or more genes are detected by PCR. In some embodiments, the mutations in one or more genes are detected by whole exome sequencing. In some embodiments, the mutations in one or more genes are detected by Sanger sequencing. In some embodiments, the mutations in one or more genes are detected by whole genome sequencing.

In certain embodiments, detection of the genetic mutation involves the use of a probe/primer in a polymerase chain reaction (PCR) (see, e.g., U.S. Pat. Nos. 4,683,195, 4,683,202 and 5,854,033), such as real-time PCR, COLD-PCR, anchor PCR, recursive PCR or RACE PCR, or, alternatively, in a ligation chain reaction (LCR) (see, e.g., Landegran et al. (1988) Science 241:1077; Prodromou and Pearl (1992) Protein Eng. 5:827; and Nakazawa et al. (1994) Proc. Natl. Acad. Sci. USA 91:360), the latter of which can be particularly useful for detecting point mutations in a myeloproliferative disorder-associated gene (e.g. JAK2. MPL, CALR, ASXL1, EZH2, SRSF2, IDH1, or IDH2) (see Abravaya et al. (1995) Nucleic Acids Res. 23:675). This method can include the steps of collecting a sample of cells from a subject, isolating nucleic acid (e.g., genomic, mRNA or both) from the cells of the sample, contacting the nucleic acid sample with one or more primers which specifically hybridize to myeloproliferative disorder-associated gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., (1990) Proc. Natl. Acad. Sci. USA 87:1874), transcriptional amplification system (Kwoh et al., (1989) Proc. Natl. Acad. Sci. USA 86:1173), Q-Beta Replicase (Lizardi et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in one or more myeloproliferative disorder-associated genes (e.g. JAK2, MPL, CALR, ASXL1, EZH2, SRSF2, IDH1, or IDH2) from a sample cell can be identified by alterations in restriction enzyme cleavage patterns. For example, sample and control DNA is isolated, optionally amplified, digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in one or more of the myeloproliferative disorder-associated genes (e.g. JAK2, MPL, CALR, ASXL1, EZH2, SRSF2, IDH1, or IDH2) described herein can be identified by hybridizing a sample and control nucleic acids, e.g., DNA or RNA, to high density arrays containing hundreds or thousands of oligonucleotides probes (Cronin et al. (1996) Human Mutation 7: 244; Kozal et al. (1996) Nature Medicine 2:753). For example, genetic mutations in a nucleic acid can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence a mycloproliferative disorder-associated gene and detect mutations by comparing the sequence of the sample gene with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147).

Other methods for detecting mutations in a myeloproliferative disorder-associated gene (e.g. JAK2, MPL, CALR, ASXL1, EZH2, SRSF2, IDH1, or IDH2) include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type sequence with potentially mutant RNA or DNA obtained from a tissue sample. The double-stranded duplexes are treated with an agent that cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286. In one embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657). According to an exemplary embodiment, a probe based on a myeloproliferative disorder-associated gene sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in myeloproliferative disorder-associated genes. For example, single strand conformation polymorphism (SSCP) may be used to detect differences in electrophoretic mobility between mutant and wild type nucleic acids (Orita et al. (1989) Proc. Natl. Acad. Sci. USA 86:2766, see also Cotton (1993) Mutat. Res. 285:125; and Hayashi (1992) Genet. Anal. Tech. Appl. 9:73). Single-stranded DNA fragments of sample and control nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis. DNA will be modified to insure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant disclosure. Oligonucleotides used as primers for specific amplification may carry the mutation of interest in the center of the molecule (so that amplification depends on differential hybridization) (Gibbs et al. (1989) Nucl. Acids Res. 17:2437) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification. In some embodiments, quantitative real-time allele-specific PCR, for example, in which allelic discrimination is enhanced by the synergistic effect of a mismatch in the −1 position, and a locked nucleic acid (LNA) at the −2 position, is used to detect and/or quantify the presence or absence of a mutation (Nussenzveig et al. (2007) Exp Hematol. 35(1):32-8).

In certain exemplary embodiments, the level of mRNA corresponding to the myeloproliferative disorder-associated gene (e.g. JAK2, MPL, CALR, ASXL1, EZH2, SRSF2, IDH1, or IDH2) can be determined either by in situ and/or by m vitro formats in a biological sample using methods known in the art. In some embodiments, the level of an MPD-associated miRNA can be determined either by in situ and/or by in vitro formats in a biological sample using methods known in the art. In some embodiments, an MPD-associated mRNA or miRNA is present in an exosome. Many expression detection methods use isolated RNA. For in vitro methods, any RNA isolation technique that does not select against the isolation of mRNA can be utilized for the purification of RNA from blood cells (see, e.g., Ausubel et al, ed., Current Protocols in Molecular Biology, John Wiley & Sons, New York 1987 1999). Additionally, large numbers of cells and/or samples can readily be processed using techniques well known to those of skill in the art, such as, for example, the single-step RNA isolation process of Chomezynski (1989, U.S. Pat. No. 4,843,155).

Isolated mRNA can be used in hybridization or amplification assays that include, but are not limited to, Southern or Northern analyses, polymerase chain reaction analyses and probe arrays. In certain exemplary embodiments, a diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a myeloproliferative disorder-associated gene. Other suitable probes for use in the diagnostic assays of the disclosure are described herein. Hybridization of an mRNA with the probe indicates that the mutation in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in a gene chip array. A skilled artisan can readily adapt known mRNA detection methods for use in detecting the level of mRNA encoded by a myeloproliferative disorder-associated gene.

An alternative method for determining the level of mRNA corresponding to a myeloproliferative disorder-associated gene (e.g. JAK2, MPL, CALR, ASXL1, EZH2, SRSF2, IDH1, or IDH2) in a sample involves the process of nucleic acid amplification, e.g., by rtPCR (the experimental embodiment set forth in U.S. Pat. Nos. 4,683,195 and 4,683,202), quantitative real-time allele-specific PCR (Borowczyk et al. (2015) Thromb Res. 135(2):272-80). COLD-PCR (Li et al. (2008) Nat. Med. 14:579), ligase chain reaction (Barany. 1991, Proc. Natl. Acad. Sci. USA, 88:189), self sustained sequence replication (Guatelli et al., 1990, Proc. Natl. Acad. Sci. USA 87:1874), transcriptional amplification system (Kwoh et al. (1989) Proc. Natl. Acad. Sci. USA 86:1173), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the sample (e.g., a bodily fluid (e.g., blood cells)) prior to detection. In such methods, a cell or tissue sample is prepared/processed using known histological methods. The sample is then immobilized on a support, typically a glass slide, and then contacted with a probe that can hybridize to an mRNA of the disclosure.

Determinations may be based on the normalized expression level of a myeloproliferative disorder-associated gene (e.g. JAK2, MPL, CALR, ASXL1, EZH2, SRSF2, IDH1, or IDH2). Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in a patient sample from one source to a patient sample from another source.

According to the disclosure, the presence or absence of myeloproliferative disorder-associated mutations can also be determined by analyzing the proteins (e.g. JAK2, MPL, CALR, ASXL1, EZH2, SRSF2, IDH1, or IDH2) encoded by the mutated genes. Detection of mutations at the protein level can be detected by any method well known in the field. In one embodiment, detection of mutation in the myeloproliferative disorder-associated protein is carried out by isolating the protein and subjecting it to amino acid sequence determination. This may require fragmenting the protein by proteolytic or chemical means prior to sequencing. Methods of determining an amino acid sequence are well known in the art.

Detection of mutated proteins can be accomplished using, for example, antibodies, aptamers, ligands; substrates, other proteins or protein fragments, other protein-binding agents, or mass spectrometry analysis of fragments. Preferably, protein detection agents are specific for the mutated proteins of the present disclosure and can therefore discriminate between a mutated protein and the wild-type protein or another variant form. This can generally be accomplished by, for example, selecting or designing detection agents that bind to the region of a protein that differs between the variant and wild-type protein.

One preferred agent for detecting a mutated protein is an antibody capable of selectively binding to a variant form of the protein. Antibodies capable of distinguishing between wild-type and mutated proteins may be created by any suitable method known in the art. The antibodies may be monoclonal or polyclonal antibodies, single chain or double chain, chimeric or humanized antibodies or portions of immunoglobulin molecules containing the portions known in the state of the art to correspond to the antigen binding fragments.

Methods for manufacturing polyclonal antibodies are well known in the art. Typically, antibodies are created by administering (e.g., via subcutaneous injection) the mutated protein immunogenic fragment containing the mutation to white New Zealand rabbits. The antigen (e.g. mutant JAK2, MPL, CLR, ASXL1, EZH2, SRSF2, IDH1 or IDH2) is typically injected at multiple sites and the injections are repeated multiple times (e.g., approximately bi-weekly) to induce an immune response. Desirably, the rabbits are simultaneously administered an adjuvant to enhance immunity. The polyclonal antibodies are then purified from a serum sample, for example, by affinity chromatography using the same antigen to capture the antibodies. The antibodies can be made specific to the mutation by removing antibodies cross-reacting with native protein.

In vitro methods for detection of the mutated proteins (e.g. mutant JAK2, MPL, CLR, ASXL1, EZH2, SRSF2, IDH1 or IDH2) also include, for example, enzyme linked immunosorbent assays (ELISAs), radioimmunoassavs (RIA), Western blots, immunoprecipitations, immunofluorescence, and protein arrays chips (e.g., arrays of antibodies or aptamers). For further information regarding immunoassays and related protein detection methods, see Current Protocols in Immunology, John Wiley & Sons, N.Y., and Hage, “Immunoassays”, Anal Chem: 1999 Jun. 15; 71(12):294R-304R. Additional analytic methods of detecting amino acid variants include, but are not limited to, altered electrophoretic mobility (e.g., 2-dimensional electrophoresis), altered tryptic peptide digest, altered JAK2 kinase activity in cell-based or cell-free assay, alteration in ligand or antibody-binding pattern, altered isoelectric point, and direct amino acid sequencing.

The disclosure also encompasses kits for detecting the presence of one or more mutations in one or more myeloproliferative disorder-associated genes (e.g. JAK2, MPL, CALR, ASXL1, EZH2, SRSF2, IDH1, or IDH2) in a biological sample. For example, the kit can comprise a labeled compound or agent capable of detecting marker genomic DNA, polypeptide, protein mRNA, and the like in a biological sample; and means for determining the presence or absence of the mutation in the sample. The compound or agent can be packaged in a suitable container. The kit can further comprise instructions for using the kit to detect the mutations in the marker peptide or nucleic acid.

### Biological Sample Collection and Preparation

The methods and compositions of this disclosure may be used to detect mutations in a myeloproliferative disorder-associated gene (e.g. JAK2, MPL, CALR, ASXL1, E7H2, SRSF2, IDH1, or IDH2) and/or myeloproliferative disorder-associated protein (e.g. JAK2, MPL, CALR, ASXL1, EZH2, SRSF2, IDH1, or IDH2) using a biological sample obtained from an individual. Methods of obtaining test samples are well known to those of skill in the art and include, but are not limited to, aspirations, tissue sections, drawing of blood or other fluids, surgical or needle biopsies, and the like. The test sample may be obtained from an individual or patient diagnosed as having a myeloproliferative disorder or suspected being afflicted with a myeloproliferative disorder or undergoing therapy for a myeloproliferative disorder. The test sample may be a cell-containing liquid or a tissue. Samples may include, but are not limited to, amniotic fluid, biopsies, blood, blood cells, bone marrow, fine needle biopsy samples, peritoneal fluid, amniotic fluid, plasma, pleural fluid, saliva, semen, serum, tissue or tissue homogenates, frozen or paraffin sections of tissue. Samples may also be processed, such as sectioning of tissues, fractionation, purification, or cellular organelle separation. If necessary, the sample may be collected or concentrated by centrifugation and the like. The cells of the sample may be subjected to lysis, such as by treatments with enzymes, heat, surfactants, ultrasonication, or a combination thereof. The lysis treatment is performed in order to obtain a sufficient amount of nucleic acid derived from the individual's cells to detect using for example, polymerase chain reaction. Alternatively, mutations in the myeloproliferative disorder-associated gene may be detected using an acellular bodily fluid according to the methods described in U.S. patent application Ser. No. 11/408,241 (Publication No. US 2007-0248961), hereby incorporated by reference.

Methods of plasma and serum preparation are well known in the art. Either “fresh” blood plasma or serum, or frozen (stored) and subsequently thawed plasma or serum may be used. Frozen (stored) plasma or serum should optimally be maintained at storage conditions of −20 to −70 degrees centigrade until thawed and used. “Fresh” plasma or serum should be refrigerated or maintained on ice until used, with nucleic acid (e.g., RNA, DNA or total nucleic acid) extraction being performed as soon as possible. Exemplary methods are described below. Blood can be drawn by standard methods into a collection tube, typically siliconized glass, either without anticoagulant for preparation of serum, or with EDTA, sodium citrate, heparin, or similar anticoagulants for preparation of plasma. If preparing plasma or serum for storage, although not an absolute requirement, it is preferable that plasma or serum is first fractionated from whole blood prior to being frozen. This reduces the burden of extraneous intracellular RNA released from lysis of frozen and thawed cells which might reduce the sensitivity of the amplification assay or interfere with the amplification assay through release of inhibitors to PCR such as porphyrins and hematin. “Fresh” plasma or serum may be fractionated from whole blood by centrifugation, using gentle centrifugation at 300-800 times gravity for five to ten minutes, or fractionated by other standard methods. High centrifugation rates capable of fractionating out apoptotic bodies should be avoided. Since heparin may interfere with RT-PCR, use of heparinized blood may require pretreatment with heparanase, followed by removal of calcium prior to reverse transcription. Imai, H., et al., J. Virol. Methods 36:181-184, (1992). Thus, EDTA is a suitable anticoagulant for blood specimens in which PCR amplification is planned.

### Nucleic Acid Extraction and Amplification

The nucleic acid to be amplified may be from a biological sample such as an organism, cell culture, tissue sample, and the like. The biological sample can be from a subject which includes any animal, preferably a mammal. A preferred subject is a human, which may be a patient presenting to a medical provider for diagnosis or treatment of a disease. The biological sample may be obtained from a stage of life such as a fetus, young adult, adult, and the like. Particularly preferred subjects are humans being tested for a mutation in a myeloproliferative disorder-associated gene (e.g. JAK2, MPL, CALR, ASXL1, EZH2, SRSF2, IDH1, or IDH2).

Various methods of extraction are suitable for isolating the DNA or RNA. Suitable methods include phenol and chloroform extraction. See Maniatis et al., Molecular Cloning, A Laboratory Manual, 2d. Cold Spring Harbor Laboratory Press, page 16.54 (1989). Numerous commercial kits also yield suitable DNA and RNA including, but not limited to, QIAamp™ mini blood kit, Agencourt Genfind™, Roche Cobast® Roche MagNA Pure® or phenol:chloroform extraction using Eppendorf Phase Lock Gels®, and the NucliSens extraction kit (Biomerieux, Marcy l'Etoile, France). In other methods, mRNA may be extracted from patient blood/bone marrow samples using MagNA Pure LC mRNA HS kit and Mag NA Pure LC Instrument (Roche Diagnostics Corporation, Roche Applied Science. Indianapolis, Ind.).

Numerous methods are known in the art for isolating total nucleic acid, DNA and RNA from blood, serum, plasma and bone marrow or other hematopoietic tissues. In fact, numerous published protocols, as well as commercial kits and systems are available. By way of example but not by way of limitation, examples of such kits, systems and published protocols are described below. Commercially available kits include Qiagen products such as the QiaAmp DNA Blood MiniKit (Cat.#51104, Qiagen, Valencia, Calif.), the QiaAmp RNA Blood MiniKit (Cat.#52304, Qiagen, Valencia, Calif.); Promega products such as the Wizard Genomic DNA Kit (Cat.# A1620, Promega Corp. Madison, Wis.), Wizard SV Genomic DNA Kit (Cat.# A2360, Promega Corp. Madison, Wis.), the SV Total RNA Kit (Cat.# X3100, Promega Corp. Madison, Wis.), PolyATract System (Cat.# Z5420. Promega Corp. Madison, Wis.), or the PurYield RNA System (Cat.#23740, Promega Corp. Madison, Wis.).

Extraction of RNA from Plasma or Serum

Plasma RNA is highly sensitive and may replace DNA-based testing because of the relative abundance of the RNA and the ease in detecting deletions such as, for example, deletion of JAK2 exon 14. Circulating extracellular deoxyribonucleic acid (DNA), including tumor-derived or associated extracellular DNA, is also present in plasma and serum. See Stroun. M., et al., Oncology 46:318-322, (1989). Since this DNA will additionally be extracted to varying degrees during the RNA extraction methods described above, it may be desirable or necessary (depending upon clinical objectives) to further purify the RNA extract and remove trace DNA prior to proceeding to further RNA analysis. This may be accomplished using DNase, for example by the method as described by Rashtchian, A., PCR Methods Applic. 4:S83-S91, (1994), as follows.

Glass beads, Silica particles or Diatom Extraction: RNA may be extracted from plasma or serum using silica particles, glass beads, or diatoms, as in the method or adaptations of Boom, R., et al., J. Clin. Micro. 28:495-503, (1990); Cheung, R. C., et al., J. Clin Micro. 32:2593-2597, (1994).

Acid Guanidinium Thiocyanate-Phenol-Chloroform Extraction: As an alternative method, RNA may be extracted from plasma or serum using the Acid Guanidinium Thiocyanate-Phenol-chloroform extraction method described by Chomezynski, P, and Sacchi, N., Analytical Biochemistry 162:156-159, (1987), as follows.

Alternative Nucleic Acid Extraction Methods: Alternative methods may be used to extract RNA from body fluids including but not limited to centrifugation through a cesium chloride gradient, including the method as described by Chirgwin, J. M., et al., Biochemistry 18:5294-5299, (1979), and co-precipitation of extracellular RNA from plasma or serum with gelatin, such as by adaptations of the method of Fournie, G. J., et al., Analytical Biochemistry 158:250-256, (1986), to RNA extraction.

### Cytogenetic Analyses

Metaphase cytogenetic analysis may be carried out by standard karyotype methods, fluorescence in situ hybridization (FISH), spectral karyotyping or multiplex-FISH (M-FISH), multicolor FISH (mFISH), and comparative genomic hybridization), and/or in situ hybridization. It will be understood that any of the commercially available probes for conducting FISH analyses (e.g., Abbott Molecular VYSIS FISH Technology) may be employed in the methods of the disclosure. In one embodiment, the method includes: contacting a sample, e.g., a chromosomal sample or a fractionated, enriched or otherwise pre-treated sample) obtained from the subject with a probe (e.g., a probes specific for the desired sequence) under conditions suitable for hybridization, and determining the presence or absence of one or more of the abnormalities in the gene (e.g., genomic DNA in chromosomal regions associated with cytogenetic abnormalities). In some embodiments, cytogenetic analysis is performed on metaphases obtained from unstimulated bone marrow aspirate cultures using standard techniques known in the art (Tam et al. Blood 113(18): 4171-4178). The method can, optionally, include enriching a sample for the gene or gene product. In some embodiments, cytogenetic analysis is carried out according to the International System for Human Cytogenetic Nomenclature. (Cytogenetic and genome research. 2013. Prepublished on 2013 Jul. 3 as DOI 10.1159/000353118).

In certain embodiments of any of the foregoing or following, the method of treatment may be effective to improve one or more manifestations of the MPD, such as bone marrow fibrosis or other manifestations described herein. In certain embodiments, the method of treatment is effective to both decrease allele burden and to improve one or more additional manifestations. In certain embodiments, improvement over time is evaluated by determining change in one or more manifestations, such as bone marrow fibrosis.

### Methods of Administration

In one aspect, the disclosure provides methods for treating a myeloproliferative disorder in a patient by administering a therapeutically effective amount of an SAP protein of the disclosure to a patient in need thereof. In one aspect, the disclosure provides methods for treating a myeloproliferative disorder in a patient carrying a mutation associated with the myeloproliferative disorder (e.g., a mutation in JAK2, MPL, CALR, ASXL1, EZH2, SRSF2, IDH1, or IDH2) by administering a therapeutically effective amount of an SAP protein of the disclosure. The dosage and frequency of treatment can be determined by one skilled in the art and will vary depending on the symptoms, age and body weight of the patient, and the nature and severity of the disorder to be treated or prevented. The present disclosure has identified dosing regimens that are effective in treating myelofibrosis.

In one aspect, the disclosure provides methods for treating a myeloproliferative disorder in a patient carrying a mutation associated with the myeloproliferative disorder (e.g. a mutation in JAK2, MPL, CALR, ASXL1, EZH2, SRSF2, IDH1, or IDH2) by administering a therapeutically effective amount of an SAP protein of the disclosure to a patient in need thereof according to a dosage regimen effective to reduce mutant allele burden. In some embodiments, the patient, prior to administration of the SAP protein, was not receiving any therapy other than transfusions. In some embodiments, the patient is anemic or thrombocytopenic.

As used herein, the term “dosage regimen” encompasses both the dose or dosage (i.e., the amount of the SAP protein) and the dosing schedule (i.e., the frequency of administration or intervals between successive doses of the SAP protein).

Administration of an SAP protein of the disclosure, singly or in combination with another agent such as an additional anti-cancer agent, according to either a weekly dosing schedule or a less frequent dosing schedule (e.g., less than weekly, such as every 4 weeks), resulted in significant improvements in myeloproliferative disorder symptoms. Moreover, the methods of the disclosure are also based on the finding that an SAP protein of the disclosure was well tolerated both alone and in combination with another anti-cancer therapeutic, with no evidence of clinically significant myelosuppression induced by the SAP treatment, e.g., treatment-related myelosuppression.

In certain aspects, the disclosure provides methods for treating a myeloproliferative disorder in a patient by administering to a patient in need thereof an SAP protein of the disclosure in an amount effective to reduce mutant allele burden. In certain aspects, the disclosure provides methods for treating a myeloproliferative disorder in a patient by administering to a patient in need thereof an SAP protein of the disclosure in an amount effective to improve the functioning of an affected organ. Improvement in function may be evaluated by, for example, evaluating a decrease in organ fibrosis, an improvement in platelet levels, and/or an increase in hemoglobin. In some embodiments, the fibrotic organ is the bone marrow. In some embodiments, the myeloproliferative disorder is myelofibrosis.

In some embodiments, an SAP protein is administered to a patient once or twice per day, once or twice per week, once or twice per month, or once per month, or just prior to or at the onset of symptoms. In some embodiments, the SAP protein is administered to a patient more frequently at the onset of the treatment regimen (e.g., every other day for the first week and once every four weeks thereafter). In some embodiments, an SAP protein is administered to a patient with PV or ET who has not yet developed fibrosis, to prevent development of fibrosis. In some embodiments, an SAP protein is administered to a patient who has been determined to carry a mutation or cytogenetic abnormality associated with the myeloproliferative disorder (e.g., a mutation in one or more of JAK2, MPL, CALR, ASXL1, EZH2, SRSF2, IDH1, or IDH2) prior to the onset of symptoms.

Dosages may be readily determined by techniques known to those of skill in the art or as taught herein. Toxicity and therapeutic efficacy of an SAP protein may be determined by standard pharmaceutical procedures in experimental animals, for example, determining the LD₅₀ and the ED₅₀. The ED₅₀ (Effective Dose 50) is the amount of drug required to produce a specified effect in 50% of an animal population. The LD₅₀ (Lethal Dose 50) is the dose of drug which kills 50% of a sample population.

In certain aspects, an SAP protein is administered as a single agent for treating a myeloproliferative disorder in a subject. In certain aspects, administering a combination of an SAP protein (e.g., a variant SAP protein of the disclosure) and an additional anti-cancer therapeutic (e.g., a chemotherapeutic agent or a kinase inhibitor) optionally provides synergistic effects for treating a myeloproliferative disorder, e.g., myelofibrosis in a subject. In some embodiments, the SAP protein and the additional anti-cancer therapeutic (e.g., a chemotherapeutic agent or a kinase inhibitor) act on different aspects of the disease. Such an approach, combination or co-administration of the two types of agents, can be useful for treating individuals suffering from myeloproliferative disorders who do not respond to or are resistant to currently-available therapies. The combination therapy provided herein is also useful for improving the efficacy and/or reducing the side effects of currently-available therapies for individuals who do respond to such therapies.

In certain embodiments, the disclosure provides methods of treating myelofibrosis in a patient carrying a mutation in one or more myelofibrosis-associated genes (e.g. JAK2, MPL, CALR, ASXL1, EZH2, SRSF2, IDH1, or IDH2) and/or one or more MPD-associated cytogenetic abnormalities, comprising administering an amount of an SAP protein, to a subject in need thereof according to a dosing regimen (e.g., a dose and dosing schedule) and/or dosing schedule effective to ameliorate one or more symptoms of myelofibrosis and reduce mutant allele burden, wherein the subject in need thereof is not receiving therapy for myelofibrosis other than transfusions. In some embodiments, the subject is anemic or thrombocytopenic. In some embodiments, the methods of the disclosure do not induce treatment-related myelosuppression [e.g., the SAP protein does not induce clinically significant myelosuppression and/or does not increase (and may even decrease) myelosuppression present at baseline]. In other words, in certain embodiments, methods of the present disclosure do not induce or result in worsening of myelosuppression in comparison to, for example, that observed prior to initiation of treatment. Myelosuppression may be assessed according to the Common Terminology for Coding of Adverse Events (CTCAE) on a scale of Grade 0-Grade 5 (See National Cancer Institute Common Terminology Criteria for Adverse Events v4.0, NCI, NIH, DHHS. May 29, 2009 NIH publication #09-7473). In some embodiments, one or more measures of myelosuppression, such as anemia, do not deteriorate (e.g., from a Grade 3 to Grade 4 adverse event; from a Grade 2 to Grade 3 or 4 adverse event; from a Grade 1 to a Grade 2, 3, or 4 adverse event; from a Grade 0 to a Grade 1, 2, 3, or 4 adverse event) as a result of treatment.

In one aspect, the disclosure provides a method for treating a myeloproliferative disorder in an individual carrying a myeloproliferative disorder-associated mutation (e.g., a mutation in JAK2, MPL, CALR, ASXL1, EZH2, SRSF2, IDH1, or IDH2 in some of his cells and/or one or more myeloproliferative disorder-associated cytogenetic abnormalities, comprising: a) obtaining a sample from said individual, wherein said sample comprises the nucleic acid of interest, b) evaluating a sample from the individual for the presence or absence of one or more mutations and/or cytogenetic abnormalities in the nucleic acid of interest, and c) administering an SAP protein of the disclosure to the individual carrying a myeloproliferative disorder-associated mutation and/or cytogenetic abnormality (e.g., a mutation in JAK2, MPL, CALR, ASXL1, EZH2, SRSF2, IDH1, or IDH2).

In one aspect, the disclosure provides a method for treating a myeloproliferative disorder in an individual carrying a myeloproliferative disorder-associated mutation (e.g., a mutation in JAK2, MPL, CALR, ASXL1, EZH2, SRSF2, IDH1, or IDH2) in some his cells and/or one or more myeloproliferative disorder-associated cytogenetic abnormalities, comprising: a) obtaining a sample from said individual, wherein said sample comprises JAK2 nucleic acid, b) evaluating a sample from the individual for the presence or absence of one or more mutations in JAK2 nucleic acid, and c) administering an SAP protein of the disclosure according to a dosage regimen effective to reduce JAK2 mutant allele burden.

In one aspect, the disclosure provides a method for reducing mutant allele burden in a patient having a myeloproliferative disorder, comprising administering an SAP protein of the disclosure.

In one aspect, the disclosure provides a method of determining the efficacy of treatment in an individual diagnosed with a myeloproliferative disease, the method comprising: (a) determining the presence of one or more mutations in JAK2, MPL, CALR, ASXL1, EZH2, SRSF2, IDH1, or IDH2 nucleic acid sample; b) administering an SAP protein of the disclosure and (c) identifying the treatment as having been effective when the mutant allele burden of one or more mutations present in the nucleic acid sample is decreased. In some embodiments, eradication of a pre-existing abnormality (e.g., the allele burden of a mutation in JAK2, MPL, CALR, ASXL1, EZH2, SRSF2, IDH1, or IDH2) is considered to be a complete response while a >50% reduction in allele burden is considered to be a partial response. In some embodiments, partial response only applies to patients with at least 20% mutant allele burden at baseline (Tefferi et al. Blood 2013, 122:1395-1398).

In one aspect, the disclosure provides a method of determining the efficacy of treatment in an individual diagnosed with a myeloproliferative disease, the method comprising: (a) determining the presence of one or more cytogenetic abnormalities, b) administering an SAP protein of the disclosure and (c) identifying the treatment as having been effective when the one or more cytogenetic abnormalities are decreased. In some embodiments, eradication of a pre-existing abnormality (e.g., a cytogenetic abnormality) is considered to be a complete response while a >50% reduction in abnormal metaphases is considered to be a partial response. In some embodiments, partial response only applies to patients with at least 10 abnormal metaphases at baseline (Tefferi et al. Blood 2013, 122:1395-1398).

In another aspect, the disclosure provides a method for selecting therapy for an individual with a hematopoietic disorder comprising evaluating a sample containing nucleic acids from the individual for the presence or absence of one or more mutations in JAK2, MPL, CALR, ASXL1, EZH2, SRSF2, IDH1, or IDH2 nucleic acid and selecting the therapy based on the presence of the one or more mutations.

One or more of the following determinations may be used to select a treatment plan: determining the presence or absence of a specific myeloproliferative disorder-associated mutation (e.g. mutations in JAK2, MPL, CALR, ASXL1, EZH2, SRSF2, IDH1, or IDH2), determining the zygosity status of the sample, and determining the ratio of mutant to wild-type nucleic acid or mRNA in the sample (e.g. mutant to wild-type ratios for JAK2, MPL, CALR, ASXL1, EZH2, SRSF2, IDH1, or IDH2). For example, patients found to carry a specific JAK2, MPL, CALR, ASXL1, EZH2, SRSF2, IDH1, or IDH2 mutation by the methods of the disclosure may be recommended for treatment with an SAP protein of the disclosure, or detection of the mutation may be used to measure the effectiveness of treatment with an SAP protein of the disclosure. Similarly, methods of the disclosure may be used to treat patients who are asymptomatic for MPD, for example patients who are in the very early stages of an MPD. Mutations may also be detected in MPD patients who are undergoing treatment, if the ratio of mutant to wild-type nucleic acid or the zygosity status of the sample changes during treatment, a different diagnosis may be made.

One or more of the following determinations may be used to treat a patient with an SAP protein of the disclosure: determining the presence or absence of a specific myeloproliferative disorder-associated mutation (e.g. mutations in JAK2, MPL, CALR, ASXL1, EZH2, SRSF2, IDH1, or IDH2), determining the zygosity status of the sample, and determining the ratio of mutant to wild-type nucleic acid in a sample (e.g. mutant to wild-type ratios for JAK2, MPL, CALR, ASXL1, EZH2, SRSF2, IDH1, or IDH2). A physician or treatment specialist may administer, forego or alter a treatment or treatment regime based on one or more of the determinations. Alternatively, a physician or treatment specialist may decide to maintain the treatment region as is based on one or more of the determinations. Further, the number of cells carrying the mutation may change during the course of an MPD and monitoring the ratio, the zygosity status, and/or the presence or absence of a mutation may be an indication of disease status or treatment efficacy. For example, treatment may reduce the number of mutant cells, or the disease may become worse with time, and the number of diseased cells may increase. Additionally, one or more of the determinations may aid in patient prognosis and quality of life decisions. For example, decisions about whether to continue or for how long to continue a painful, debilitating treatment such as chemotherapy may be made.

The zygosity status and the ratio of wild-type to mutant nucleic acid in a sample may be determined by methods known in the art including sequence-specific, quantitative detection methods. Other methods may involve determining the area under the curves of the sequencing peaks from standard sequencing electropherograms, such as those created using ABI Sequencing Systems, (Applied Biosystems, Foster City Calif.). For example, the presence of only a single peak such as a “G” on an electropherogram in a position representative of a particular nucleotide is an indication that the nucleic acids in the sample contain only one nucleotide at that position, the “G.” The sample may then be categorized as homozygous because only one allele is detected. The presence of two peaks, for example, a “G” peak and a “T” peak in the same position on the electropherogram indicates that the sample contains two species of nucleic acids; one species carries the “G” at the nucleotide position in question, the other carries the “T” at the nucleotide position in question. The sample may then be categorized as heterozygous because more than one allele is detected.

The sizes of the two peaks may be determined (e.g., by determining the area under each curve), and a ratio of the two different nucleic acid species may be calculated. A ratio of wild-type to mutant nucleic acid (e.g. mutant to wild-type ratios for JAK2, MPL, CALR, ASXL1, EZH2, SRSF2, IDH1, or IDH2) may be used to monitor disease progression, determine treatment, or to make a diagnosis. For example, the number of cancerous cells carrying a specific JAK2, MPL, CALR, ASXL1, EZH2, SRSF2, IDH1, or IDH2 mutation may change during the course of an MPD. If a base line ratio is established early in the disease, a later determined higher ratio of mutant nucleic acid relative to wild-type nucleic acid may be an indication that the disease is becoming worse or a treatment is ineffective; the number of cells carrying the mutation may be increasing in the patient. A lower ratio of mutant relative to wild-type nucleic acid may be an indication that a treatment is working or that the disease is not progressing, the number of cells carrying the mutation may be decreasing in the patient.

In some embodiments, the subject carries mutations in one or more myeloproliferative disorder-associated genes such as but not limited to JAK2, MPL, CALR, ASXL1, EZH2, SRSF2, IDH1, or IDH2. In some embodiments, the subject has a mutation at codon 617 of JAK2. In some embodiments, the subject has a mutation in exon 12 or exon 14 of JAK2. In some embodiments, the subject has a mutation at codon 515 of MPL. In some embodiments, the subject has a W515L, W515K, W515A, or W515R amino acid substitution in MPL. In some embodiments, the subject has a mutation in exon 10 of MPL. In some embodiments, the subject has a mutation in exon 9 of CALR. In some embodiments, the subject has a mutation in exon 12 of ASXL1. In some embodiments, the subject has a mutation in exon 4 of IDH1. In some embodiments, the subject has a mutation at codon 132 of IDH1. In some embodiments, the subject has a mutation in exon 4 of IDH2. In some embodiments, the subject has a mutation at codon 140 of IDH2. In some embodiments, the subject has a mutation at codon 172 of IDH2.

In certain embodiments of any of the above aspects of the disclosure, the myeloproliferative disease is polycythemia vera, essential thrombocythemia, myelofibrosis, or an unclassified myeloproliferative disease. In some embodiments, the myelofibrosis is primary myleofibrosis, post-PV myelofibrosis, or post-ET myelofibrosis.

In some embodiments, evaluating or determining the presence or absence of one or more mutations in a nucleic acid sample of interest includes performing allele-specific PCR. In other embodiments of any of the above aspects of the disclosure, evaluating or determining the presence or absence of one or more mutations in a nucleic acid sample of interest includes amplifying the nucleic acid of interest and performing direct sequencing analysis of the amplified nucleic acid. Other suitable detection methodologies include primer extension reaction, and protein sequencing. In certain embodiments of the above aspects, evaluating a sample or determining the presence or absence of one or more mutations in a myeloproliferative disorder-associated polypeptide includes using an antibody that specifically binds to the mutated JAK2 polypeptide. In some embodiments of any of the above aspects of the disclosure, the nucleic acid and/or polypeptide sample is from a suitable biological sample including, for example, whole blood (i.e., JAK2 nucleic acid being extracted from the cellular fraction), plasma, serum, and tissue samples (e.g., biopsy and paraffin-embedded tissue).

In one aspect, the disclosure provides methods for treating myeloproliferative disorders (e.g., myelofibrosis) by administering an SAP protein in combination with one or more additional agents such as another anti-cancer therapeutic. As used herein, “in combination with” or “conjoint administration” refers to any form of administration such that the one or more additional agents is still effective in the body (e.g., the two agents, the three agents, the four agents, etc. are simultaneously effective in the patient, which may include synergistic effects of the two compounds). Effectiveness may not correlate to measurable concentration of the agent in blood, serum, or plasma. For example, the different therapeutic agents can be administered either in the same formulation or in separate formulations, either concomitantly or sequentially, and on different schedules. Thus, an individual who receives such treatment can benefit from a combined effect of different therapeutic agents. The SAP protein can be administered concurrently with, prior to, or subsequent to, one or more other additional agents.

In general, each therapeutic agent will be administered at a dose and/or on a time schedule determined for that particular agent. The particular combination to employ in a regimen will take into account compatibility of the SAP protein with the agent and/or the desired therapeutic effect to be achieved.

Anti-cancer therapeutics of the disclosure may include, but are not limited to chemotherapy agents, antibody-based agents, kinase inhibitors (e.g., tyrosine kinase inhibitors, serine/threonine kinase inhibitors, etc.), immunomodulatory agents and biologic agents or combinations thereof. Chemotherapy agents include, but are not limited to actinomycin D, aldesleukin, alitretinoin, all-trans retinoic acid/ATRA, altretamine, amascrine, asparaginase, azacitidine, azathioprine, bacillus calmette-guerin/BCG, bendamustine hydrochloride, bexarotene, bicalutamide, bleomycin, bortezomib, busulfan, capecitabine, carboplatin, carfilzomib, carmustine, chlorambucil, cisplatin/cisplatinum, cladribine, cyclophosphamide/cytophosphane, cytabarine, dacarbazine, daunorubicin/daunomycin, denileukin diftitox, dexrazoxane, docetaxel, doxorubicin, epirubicin, etoposide, fludarabine, fluorouracil (5-FU), gemcitabine, goserelin, hydrocortisone, hydroxyurea, idarubicin, ifosfamide, interferon alfa, irinotecan CPT-11, lapatinib, lenalidomide, leuprolide, mechlorethamine/chlormethine/mustine/HN2, mercaptopurine, methotrexate, methylprednisolone, mitomycin, mitotane, mitoxantrone, octreotide, oprelvekin, oxaliplatin, paclitaxel, pamidronate, pazopanib, pegaspargase, pegfilgrastim, PEG interferon, pemetrexed, pentostatin, phenylalanine mustard, plicamycin/mithramycin, prednisone, prednisolone, procarbazine, raloxifene, romiplostim, sargramostim, streptozocin, tamoxifen, temozolomide, temsirolimus, teniposide, thalidomide, thioguanine, thiophosphoamide/thiotepa, thiotepa, topotecan hydrochloride, toremifene, tretinoin, valrubicin, vinblastine, vincristine, vindesine, vinorelbine, vorinostat, zoledronic acid, or combinations thereof. Antibody-based agents include, but are not limited to alemtuzumab, bevacizumab, cetuximab, fresolimumab, gemtuzumab ozogamicin, ibritumomab tiuxetan, ofatumumab, panitumumab, rituximab, tositumomab, trastuzumab, trastuzumab DM1, and combinations thereof. Immunomodulatory compounds include, but are not limited to small organic molecules that inhibit TNFα, LPS induced monocyte IL1β, IL12, and IL6 production. In some embodiments, immunomodulatory compounds include but are not limited to methotrexate, leflunomide, cyclophosphamide, cyclosporine A, minocycline, azathioprine, an antibiotic (e.g., tacrolimus), methylprednisolone, a corticosteroid, a steroid, mycophenolate mofetil, rapamycin, mizoribine, deoxyspergualin, brequinar, a T cell receptor modulator, or a cytokine receptor modulator, and a Toll-like receptor (TLR) agonist. In some embodiments, immunomodulatory compounds include 5,6-dimethylxanthenone-4-acetic acid (DMXAA), thalidomide, lenalidomide, pomalidomide, lactoferrin, polyadenosine-polyuridylic acid (poly AU), rintatolimod (polyI:polyC12U; Hemispherx Biopharma), polyinosinic-polycytidylic acid stabilized with poly-L-lysine and carboxymethylcellulose (Poly-ICLC. Hiltonol®), imiquimod (3M) and resiquimod (R848; 3M), unmethylated CpG dinucleotide (CpG-ODN), and ipilumumab. Biologic agents include monoclonal antibodies (MABs), CSFs, interferons and interleukins. In some embodiments, the biologic agent is IL-2, IL-3, erythropoietin, G-CSF, filgrastim, interferon alfa, alemtuzumab, bevacizumab, cetuximab, gemtuzumab ozogamicin, ibritumomab tiuxetan, ofatumumab, panitumumab, rituximab, tositumomab or trastuzumab.

Kinase inhibitors (e.g., tyrosine kinase inhibitors, serine/threonine kinase inhibitors, etc.) include, but are not limited to axitinib, bafetinib, bosutinib, cediranib, crizotinib, dasatinib, erlotinib, gefitinib, imatinib, lapatinib, neratinib, nilotinib, ponatinib, quizartinib, regorafenib, sorafenib, sunitinib, vandetanib, vatalanib, vemurafinib, and combinations thereof.

In some embodiments, the anti-cancer therapeutic is a JAK kinase inhibitor such as, but not limited to AC-430, AZD1480, baricitinib, BMS-911453, CEP-33779, CYT387, GLPG-0634, lestaurtinib, LY2784544, NS-018, pacritinib, R-348, R723, ruxolitinib, TG101348 (SAR302503), tofacitinib, and VX-509.

In certain embodiments, the anti-cancer therapeutic includes but is not limited to anti-metabolites (e.g., 5-fluoro-uracil, cytarabine, methotrexate, fludarabine and others), antimicrotubule agents (e.g., vinca alkaloids such as vincristine, vinblastine; taxanes such as paclitaxel and docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, satraplatin and CI-973), anthracyclines (e.g., doxrubicin and daunorubicin), antitumor antibiotics (e.g., mitomycin, idarubicin, adriamycin and daunomycin), topoisomerase inhibitors (e.g., etoposide and camptothecins), anti-angiogenesis agents (e.g., sunitinib, sorafenib and bevacizumab) or any other cytotoxic agents, (e.g. estramustine phosphate, prednimustine), hormones or hormone agonists, antagonists, partial agonists or partial antagonists, kinase inhibitors (such as imatinib), and radiation treatment.

Any treatment method of the disclosure may be repeated as needed or required. For example, the treatment may be done on a periodic basis. The frequency of administering treatment may be determined by one of skill in the art. For example, treatment may be administered once a week for a period of weeks, once every four weeks, or multiple times a week for a period of time (e.g., 3 times over the first week of treatment). In some embodiments, an initial loading dose period is followed by a maintenance dose. In some embodiments, the loading dose is periodically repeated. In some embodiments, the initial loading dose period includes administering the treatment multiple times a week (e.g., 3 times over the first week of treatment). In some embodiments, the loading dose may be repeated every other week, every month, every two months, every 3 months, or every 6 months, or as needed, with or without continued periodic dosing between loading doses. Generally, the amelioration of the cancer-associated fibrosis persists for some period of time, preferably at least months, but maintenance of the anti-fibrotic effect and/or prevention of recurrence of fibrosis may require continued periodic dosing of an SAP protein over an unlimited period of time. Over time, the patient may experience a relapse of symptoms, at which point the treatments may be repeated.

In certain aspects, methods are provided herein for treating, delaying development, and/or preventing myelofibrosis in a subject comprising administering to the subject an effective amount of an SAP protein, or a pharmaceutically acceptable salt thereof, alone or in combination with an anti-cancer therapeutic. In some embodiments, the subject has myelofibrosis. In some embodiments, the subject is at risk of developing myelofibrosis. In some embodiments, the subject is a human subject. In some embodiments, the subject has been determined carry a mutation associated with the myeloproliferative disorder (e.g., a mutation in JAK2, MPL, CALR, ASXL1, EZH2, SRSF2, IDH1, or IDH2). Any one of the formulations described herein such as capsule or unit dosage forms described herein may be used to treat a subject with myelofibrosis.

Myelofibrosis that may be treated by the methods described herein includes primary myelofibrosis (PMF) and secondary myelofibrosis (e.g., myelofibrosis arising from antecedent polycythemia vera (post-PV MF) or essential thrombocythemia (post-ET MF)). Myelofibrosis that may be treated by the methods described herein also includes myelofibrosis of high risk, intermediate risk such as intermediate risk level 1 or intermediate risk level 2, and low risk. Methods for diagnosing various types of myelofibrosis are known in the art. See, e.g., Cervantes et al., Blood 2009, 113(13):2895-901. In some embodiments, a dynamic prognostic model that accounts for modifications to the risk profile after diagnosis may prove useful. See, e.g., Passamonti et al., Blood 2010, 115:1703-1708. In some embodiments, the subject has palpable splenomegaly. In some embodiments, the subject with myelofibrosis has spleen of at least 5 cm below costal margin as measured by palpation. In some embodiments, the subject has anemia and/or thrombocytopenia and/or leukopenia. In some embodiments, the subject does not have anemia or thrombocytopenia or leukopenia. In some embodiments, the subject is transfusion dependent. In some embodiments, the subject is not transfusion dependent. In some embodiments, the subject has a pathologically confirmed diagnosis of PMF as per the WHO diagnostic criteria or post ET/PV MF, including the presence of at least Grade 2 marrow fibrosis with intermediate −1, intermediate −2, or high risk disease according to the IWG-MRT Dynamic International Prognostic Scoring System. In some embodiments, the subject has a pathologically confirmed diagnosis of PMF as per the WHO diagnostic criteria or post ET/PV MF, with Grade 0 or 1 bone marrow fibrosis and low risk, intermediate −1, intermediate −2, high risk, or low risk disease according to the IWG-MRT Dynamic International Prognostic Scoring System. In some embodiments, the subject has “prefibrotic” myelofibrosis. In some embodiments, the subject has PV or ET and receives an SAP protein to prevent development of myelofibrosis. In some embodiments, the subject has a mutation in JAK2, MPL, CALR, ASXL1, EZH2, SRSF2, IDH1, or IDH2.

In some embodiments, the subject has a point mutation from valine 617 to phenylalanine in the Janus kinase 2 (JAK2 kinase) (JAK2V617F) if the subject is a human, or a point mutation corresponding to the valine 617 to phenylalanine in the Janus kinase 2 (JAK2 kinase) if the subject is not a human. In some embodiments, the subject is negative for the valine 617 to phenylalanine mutation of JAK2 if the subject is a human, or negative for a mutation corresponding to the valine 617 to phenylalanine in the Janus kinase 2 (JAK2 kinase) if the subject is not a human. Whether a subject is positive or negative for JAK2V617F can be determined by a polymerase chain reaction (“PCR”) analysis using genomic DNA from bone marrow cells or blood cells (e.g., whole blood leukocytes). The PCR analysis can be an allele-specific PCR (e.g., allele-specific quantitative PCR) or PCR sequencing. See Kittur J et al., Cancer 2007, 109(11):2279-84 and McLornan D et al., Ulster Med J. 2006, 75(2): 112-9, each of which is expressly incorporated herein by reference. In some embodiments, the subject has a mutation in exon 12 or exon 14 of JAK2. In some embodiments, the subject has a mutation at codon 515 of MPL. In some embodiments, the subject has a W515L, W515K, W515A, or W515R amino acid substitution in MPL. In some embodiments, the subject has a mutation in exon 10 of MPL. In some embodiments, the subject has a mutation in exon 9 of CALR. In some embodiments, the subject has a mutation in exon 12 of ASXL1. In some embodiments, the subject has a mutation in exon 4 of IDH1. In some embodiments, the subject has a mutation at codon 132 of IDH1. In some embodiments, the subject has a mutation in exon 4 of IDH2. In some embodiments, the subject has a mutation at codon 140 of IDH2. In some embodiments, the subject has a mutation at codon 172 of IDH2.

In some embodiments, the subject treated with the methods described herein has previously received or is currently receiving another myelofibrosis therapy or treatment. In some embodiments, the subject is a non-responder to the other myelofibrosis therapy or has a relapse after receiving the other myelofibrosis therapy. The previous therapy may be a JAK2 inhibitor (e.g. INCB018424 (also known as ruxolitinib, available from Incyte), CEP-701 (lestaurtinib, available from Cephalon), or XL019 (available from Exelixis)) (See Verstovsek S., Hematology Am Soc Hematol Educ Program. 2009:636-42) or a non-JAK2 inhibitor (such as hydroxyurea). In some embodiments, the previous therapy may be JAK kinase inhibitor such as, but not limited to AC-430, AZD1480, baricitinib, BMS-911453, CEP-33779, CYT387, GLPG-0634, INCB18424, lestaurtinib, LY2784544, NS-018, pacritinib, ruxolitinib, TG101348 (SAR302503), tofacitinib. VX-509, R-348, or R723. In some embodiments, the subject has received ruxolitinib treatment for primary myelofibrosis, post-polycythemia vera myelofibrosis (Post-PV MF), post-essential thrombocythemia myelofibrosis (Post-ET MF), polycythemia vera, or essential thrombocythemia for at least three months. In some embodiments, the subject has received ruxolitinib treatment for primary myelofibrosis, post-polycythemia vera myelofibrosis (Post-PV MF), post-essential thrombocythemia myelofibrosis (Post-ET MF), polycythemia vera, or essential thrombocythemia for less than three months. In some embodiments, the subject has received ruxolitinib treatment for primary myelofibrosis, post-polycythemia vera myelofibrosis (Post-PV MF), post-essential thrombocythemia myelofibrosis (Post-ET MF), polycythemia vera, or essential thrombocythemia for at least three months. In some embodiments, at least one or more symptoms have ceased to improve on continued ruxolitinib therapy. In some embodiments, the subject is no longer responsive to ruxolitinib. In some embodiments, the subject has previously received another myelofibrosis therapy for at least 6 months, at least 5 months, at least 4 months, at least 3 months, at least 2 months, at least 1 month, at least 3 weeks, or at least 2 weeks. In some embodiments, the subject is no longer responsive to the other myelofibrosis therapy. In some embodiments, the previous therapy is an anti-cancer therapeutic described herein and the previous therapy has been discontinued upon indication of one or more elevated levels of amylase, lipase, aspartate aminotransferase (AST), alanine aminotransferase (ALT), and/or creatinine in the serum from the subject, and/or upon indication of a hematologic condition selected from the group consisting of anemia, thrombocytopenia, and neutropenia, or for any other reason based on a decision by the treating physician or the patient's request. In some embodiments, the dose of the compound in the second treatment is the same or lower than the dose in the previous therapy. In some embodiments, the subject has not received any therapy other than transfusions. In some embodiments, the subject has not received any prior therapy.

In some embodiments, the SAP protein is administered in combination with a JAK kinase inhibitor such as, but not limited to AC-430, AZD1480, baricitinib, BMS-911453, CEP-33779, CYT387, GLPG-0634, INCB18424, lestaurtinib, LY2784544, NS-018, pacritinib, ruxolitinib, TG101348 (SAR302503), tofacitinib, VX-509, R-348, or R723 (See Kontzias et al. Curr Opin Pharmacol. 2012, 12(4):464-470). In some embodiments, the SAP protein is administered in combination with an agent known to reduce the symptoms of myelofibrosis, such as, but not limited to AB0024, AZD1480, AT-9283, BMS-911543, CYT387, everolimus, givinostat, imetelstat, lestaurtinib, LY2784544, oral arsenic, NS-018, pacritinib, panobinostat, peginterferon alfa-2a, pomalidomide, pracinostat, ruxolitinib, TAK-901, and TG101438 (SAR302503) (Mesa, Leuk Lymphoma 2013, 54(2):242-251; Gupta et al. 2012, 2(3):170-186; Kucine and Levine 2011, 2(4):203-211).

The subject (such as a human) may be treated by administering the SAP protein at a dose of about 0.1 mg/kg to about 40 mg/kg. In some embodiments, the SAP protein is administered at a dose of 0.3 mg/kg. In some embodiments, the SAP protein is administered at a dose of 3 mg/kg. In some embodiments, the SAP protein is administered at a dose of 10 mg/kg. In some embodiments, the compound is administered at a dose of about any of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 8 mg/kg, 10 mg/kg, 12 mg/kg, 15 mg/kg, 18 mg/kg, 20 mg/kg, 25 mg/kg, 30 mg/kg, 35 mg/kg, or 40 mg/kg. In some embodiments, the SAP protein is administered at a dose of about 0.1-0.3, 0.3-0.5, 0.5-0.8, 0.8-1, 1-5, 5-10, 10-15, 15-20, 20-25, 25-30, 30-35, or 35-40 mg/kg. The compound may be in a capsule and/or a unit dosage form described herein. In some embodiments, the compound is administered intravenously (IV). In some embodiments, the compound is administered by injection (e.g. subcutaneous (SubQ), intramuscular (IM), intraperitoneal (IP)), by inhalation or insufflation (either through the mouth or the nose) or the administration is oral, buccal, sublingual, transdermal, nasal, parenteral or rectal. In some embodiments, the SAP protein is administered by intravenous infusion. In certain embodiments, for each dose, infusion is over a period of approximately one hour. However, longer or shorter infusion periods may be used (e.g., 30 minutes, 40 minutes, 45 minutes, 50 minutes, 55 minutes, 60 minutes, 1 hour ten minutes, 1 hour fifteen minutes, 90 minutes, and the like). When the method comprises administering an additional anti-cancer therapeutic, that therapeutic may be administered by the same route of administration or by a different route of administration. In certain embodiments, an additional anti-cancer therapeutic is administered orally.

In some embodiments, the SAP protein is administered at a dose of 0.3 mg/kg by IV infusion. In some embodiments, the SAP protein is administered at a dose of 0.3 mg/kg subcutaneously. In some embodiments, the SAP protein is administered at a dose of less than 0.3 mg/kg subcutaneously. In some embodiments, the SAP protein is administered at a dose of 3 mg/kg by IV infusion. In some embodiments, the SAP protein is administered at a dose of 10 mg/kg by IV infusion. In some embodiments, the SAP protein is administered once every four weeks. In some embodiments, an initial loading dose period is followed by a maintenance dose (e.g., three times a week for the first week and once every four weeks thereafter).

Also provided herein are methods for ameliorating one or more manifestations or symptoms associated with myelofibrosis. For example, the treatment using the methods described herein is effective in reducing mutant allele burden in one or more myelofibrosis-associated genes (e.g., JAK2, MPL, CALR, ASXL1, EZH2, SRSF2, IDH1, or IDH2). In some embodiments, the treatment using the methods described herein is effective in reducing spleen size, ameliorating constitutional symptoms (such as early satiety, fatigue, night sweats, cough, and pruritus), reducing the MPN-SAF Total Symptom Score, improving quality of life as measured by the EORTC QLQ-C30, reducing leukocytosis, reducing thrombocytosis, improving anemia, improving thrombocytopenia, improving leukopenia, reducing transfusion dependence, decreasing JAK2V617F allele burden, decreasing MPL515W allele burden, decreasing CALR mutant allele burden, decreasing ASXL1 mutant allele burden, decreasing EZH2 mutant allele burden, decreasing SRSF2 mutant allele burden, decreasing IDH1 mutant allele burden, decreasing IDH2 mutant allele burden, decrease in peripheral blood blasts, decrease in bone marrow blasts, reducing bone marrow fibrosis, inducing a change in metabolic activity as measured by FDG or FLT PET-CT scan indicative of reduction in fibrosis in the bone marrow, spleen, and/or liver, improving pruritus, improving cachexia, and/or reducing or increasing bone marrow cellularity. The reduction, decrease, amelioration, or improvement can be at least by 5, 10, 20, 30, 40, 50, 60, 70, 80, or 90% compared to the level prior to commencing treatment with the methods provided herein. In some embodiments, bone marrow fibrosis is reduced in the subject after treatment. In some embodiments, bone marrow fibrosis becomes Grade 0 after treatment. In some embodiments, bone marrow fibrosis becomes Grade 1 after treatment. In some embodiments, the bone marrow fibrosis is reduced by at least one Grade, e.g. from Grade 3 to Grade 2 or Grade 1, or from Grade 2 to Grade 1 or Grade 0. In some embodiments, the bone marrow fibrosis is reduced by a measurable percent from baseline as measured by quantitative image analysis or other quantitative means. In some embodiment, the spleen becomes non-palpable in the subject after treatment. In some embodiments, the subject has complete resolution of leukocytosis and/or thrombocytosis after treatment. In some embodiments, the subject has complete resolution of anemia, thrombocytopenia and/or leukopenia after treatment. In some embodiments, the subject becomes transfusion independent (e.g., red blood cell transfusions or platelet transfusions) after treatment. In some embodiments, the subject has a 50% reduction in transfusions. In some embodiments, the subject has a 40%-60%, 30%-70%, 40%-50%, 50%, 60% reduction in transfusions. In some embodiments, the subject has complete resolution of pruritus after treatment. In some embodiments, efficacy of the treatment will be assessed by evaluation of the overall response rate (ORR) categorized according to the International Working Group (IWG) criteria modified to include stable disease with improvement in bone marrow fibrosis by at least one grade as a response. In some embodiments, efficacy of the treatment will be assessed by evaluation of improvement in bone marrow fibrosis score by at least one grade according to the European Consensus on Grading of Bone Marrow Fibrosis. In some embodiments, efficacy of treatment will be assessed by evaluating the molecular effect on a pre-existing abnormality such as a genetic mutation. Eradication of a pre-existing abnormality (e.g., the allele burden of a mutation in JAK2, MPL, CALR, ASXL1, EZH2, SRSF2, IDH1, or IDH2) is considered to be a complete response while a >50% reduction in allele burden is considered to be a partial response. Partial response only applies to patients with at least 20% mutant allele burden at baseline (Tefferi et al. Blood 2013, 122:1395-1398). In some embodiments, efficacy of the treatment will be assessed by evaluating changes in levels of circulating plasma cytokine levels including but not limited to CRP, IL-1Ra, MIP-1β, TNFα, IL-6 and VEGF. In some embodiments, efficacy of the treatment will be assessed by evaluating changes in levels of PBMC mRNA and miRNA expression levels. In some embodiments, efficacy of the treatment will be assessed by lack of progression of PV or ET to myelofibrosis. In some embodiments, efficacy of the treatment will be assessed by lack of increase in bone marrow fibrosis by at least one grade.

In some embodiments, the treatment using the methods described herein (e.g. single agent or combination therapy using an SAP protein) is effective in reducing mutant allele burden in one or more myeloproliferative disorder-associated genes (e.g. JAK2, MPL, CALR, ASXL1, EZH2, SRSF2, IDH1, or IDH2) by at least 20%, at least 25%, at least 30%, at least 35%, at least 40%, at least 45%, at least 50%, at least 55%, at least 60%, at least 65%, or at least 70% compared to the allele burden prior to commencing treatment with the methods provided herein (e.g., compared to baseline). In some embodiments, the treatment is effective in reducing mutant allele burden by about 20-70%, about 20-60%, about 25-60%, about 25-55%, or about 25%-50%. In some embodiments, the mutant allele burden is decreased by 25% to 50%. In some embodiments, the mutant allele burden is decreased by at least 50%. In some embodiments, the treatment is effective in achieving a complete molecular response. In some embodiments, allele burden may be measured by PCR performed on nucleic acid samples extracted from blood. It would be understood by one of skill in the art that other known methods to measure allele burden may also be employed. In certain embodiments, the disclosure provides methods for decreasing allele burden in a patient in need thereof, wherein the patient in need thereof has myelofibrosis, comprising administering an amount of an SAP protein according to a dosing schedule effective to decrease allele burden by at least 25%, at least 30%, at least 35%, at least 40%, or at least 50%. In certain embodiments, the SAP protein comprises an SAP protein with glycosylation that differs from that of human SAP purified from serum, and the additional anticancer therapeutic is a JAK kinase inhibitor. In certain embodiments, allele burden is decreased by about 25-55%, by about 25-50%, or by about 25-40%. In some embodiments, the subject has a JAK2V617F mutation. In some embodiments, the subject has a mutation in exon 12 or exon 14 of JAK2. In some embodiments, the subject has a mutation at codon 515 of MPL. In some embodiments, the subject has a W515L, W515K, W515A, or W515R amino acid substitution in MPL. In some embodiments, the subject has a mutation in exon 10 of MPL. In some embodiments, the subject has a mutation in exon 9 of CALR. In some embodiments, the subject has a mutation in exon 12 of ASXL1. In some embodiments, the subject has a mutation in exon 4 of IDH1. In some embodiments, the subject has a mutation at codon 132 of IDH1. In some embodiments, the subject has a mutation in exon 4 of IDH2. In some embodiments, the subject has a mutation at codon 140 of IDH2. In some embodiments, the subject has a mutation at codon 172 of IDH2. In certain embodiments, the reduction in allele burden is seen for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in reducing spleen volume by at least 10%, at least 20%, at least 25%, at least 30%, at least 35%, at least 40%, at least 45%, at least 50%, at least 55%, at least 60%, at least 65%, or at least 70% compared to the level prior to commencing treatment with the methods provided herein (e.g., compared to baseline). In some embodiments, the treatment is effective in reducing spleen volume by at least 10%. In some embodiments, the treatment is effective in reducing spleen volume by at least 25%. In some embodiments, the treatment is effective in reducing spleen volume by at least 35%. In some embodiments, the treatment is effective in reducing spleen volume by at least 50%. In some embodiments, the treatment is effective in reducing spleen volume by about 20-70%, about 20-60%, about 25-60%, about 25-55%, or about 25%-50%. In some embodiments, spleen volume may be measured by manual palpation. It would be understood by one of skill in the art that other known methods to measure spleen volume may also be employed, such as measurement by magnetic resonance imaging. In certain embodiments, the disclosure provides methods for decreasing spleen volume in a patient in need thereof, wherein the patient in need thereof has myelofibrosis, comprising administering an amount of an SAP protein according to a dosing schedule effective to decrease spleen volume by at least 10%, at least 25%, at least 30%, at least 35%, at least 40%, at least 50%, or at least 55%. In certain embodiments, the SAP protein, comprises an SAP protein with glycosylation that differs from that of human SAP purified from serum, and the additional anticancer therapeutic is a JAK kinase inhibitor. In certain embodiments, spleen volume is decreased by about 10-25%, by about 25-55%, by about 25-50%, or by about 25-40%. In certain embodiments, the reduction in spleen volume by at least 10%, at least 25%, or at least 50% is seen for ≥12 consecutive weeks following treatment (e.g., greater than 24 weeks, greater than weeks, greater than 36 weeks, greater than 42 weeks, greater than 48 weeks).

In some embodiments, the treatment using the methods described herein (e.g. single agent or combination therapy using an SAP protein) is effective in reducing the Myeloproliferative Neoplasms Symptom Assessment Form (MPN-SAF) Total Symptom Score by at least 20%, at least 25%, at least 30%, at least 35%, at least 40%, at least 45%, at least 50%, at least 55%, at least 60%, at least 65%, or at least 70% compared to the score prior to commencing treatment with the methods provided herein. See Emanuel et al., 2012, Journal of Clinical Oncology, volume 30, number 33, pages 4098-4013, for a description and discussion of the myeloproliferative neoplasm symptom assessment form total symptom score. In some embodiments, the treatment is effective in reducing the MPN-SAF Total Symptom Score by at least 25%. In some embodiments, the treatment is effective in reducing the MPN-SAF Total Symptom Score by at least 50%. In some embodiments, the symptoms were assessed using the MPN-SAF patient reported outcome tool (Emanuel et al. 2012, Journal of Clinical Oncology 30(33): 4098-4103). In certain embodiments, the disclosure provides methods for reducing the MPN-SAF Total Symptom Score in a patient in need thereof, wherein the patient in need thereof has myelofibrosis, comprising administering an amount of an SAP protein, according to a dosing schedule effective to reduce the MPN-SAF Total Symptom Score by at least about 25%, at least 30%, at least 35%, at least 40%, at least 50%, at least 55%, or at least 60%. In certain embodiments, the SAP protein comprises an SAP protein with glycosylation that differs from that of human SAP purified from serum, and the additional anticancer therapeutic is a JAK kinase inhibitor. In certain embodiments, the MPN-SAF Total Symptom Score is reduced by about 25-60%, by about 25-55%, or by about 25-50%. In certain embodiments, the reduction in the MPN-SAF Total Symptom Score is reduced by at least 25% or at least 50% for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in improving quality of life based on the EORTC QLQ-C30 score, by at least 20%, at least 25%, at least 30%, at least 35%, at least 40%, at least 45%, at least 50%, at least 55%, at least 60%, at least 65%, or at least 70% compared to the score prior to commencing treatment with the methods provided herein. See EORTC QLQ-C30 (version 3) 1995, EORTC Quality of Life Group, for a description and discussion of the EORTC QLQ-C30 questionnaire and scoring system. In some embodiments, the treatment is effective in improving the EORTC QLQ-C30 score by at least 25%. In some embodiments, the treatment is effective in improving the EORTC QLQ-C30 score by at least 50%. In some embodiments, the score was assessed using the questions and scoring system outlined in EORTC QLQ-C30 (version 3) 1995, EORTC Quality of Life Group. In certain embodiments, the disclosure provides methods for improving the EORTC QLQ-C30 score in a patient in need thereof, wherein the patient in need thereof has myelofibrosis, comprising administering an amount of an SAP protein, according to a dosing schedule effective to improve the EORTC QLQ-C30 score by at least about 25%, at least 30%, at least 35%, at least 40%, at least 50%, at least 55%, or at least 60%. In certain embodiments, the SAP protein comprises an SAP protein with glycosylation that differs from that of human SAP purified from serum, and the additional anticancer therapeutic is a JAK kinase inhibitor. In certain embodiments, the EORTC QLQ-C30 score is improved by about 25-60%, by about 25-55%, or by about 25-50%. In certain embodiments, the improvement in the EORTC QLQ-C30 score is at least 25% or at least 50% for ≥12 consecutive weeks following treatment.

In some embodiments, the treatment using the methods described herein (e.g. single agent or combination therapy using an SAP protein) is effective in increasing hemoglobin levels by at least about 500 mg/L, 1 g/L, 2 g/L, 3 g/L, 5 g/L, 10 g/L, or 20 g/L compared to the level prior to commencing treatment with the methods provided herein (e.g., compared to baseline). In some embodiments, the treatment is effective in increasing hemoglobin levels by 500-1000 mg/L, 1-2 g/L, 2-3 g/L, or 3-5 g/L compared to the level prior to commencing treatment with the methods provided herein (e.g., compared to baseline). In some embodiments, the treatment is effective in increasing hemoglobin levels by 1 g/L. In some embodiments, the treatment is effective in increasing the hemoglobin levels to at least 80 g/L, at least 90 g/L, at least 100 g/L, at least 110 g/L, at least 120 g/L, at least 130 g/L, or at least 140 g/L. In some embodiments, the treatment is effective in increasing hemoglobin levels to at least 100 g/L. In some embodiments, the hemoglobin levels are measured as part of a routine Complete Blood Count (CBC). It would be understood by one of skill in the art that other known methods to measure hemoglobin levels may also be employed. In certain embodiments, the disclosure provides methods for increasing the hemoglobin levels in a patient in need thereof, wherein the patient in need thereof has myelofibrosis, comprising administering an amount of an SAP protein, according to a dosing schedule effective to increase the hemoglobin levels by at least about 500 mg/L, 1 g/L, 2 g/L, 3 g/L, or 5 g/L. In certain embodiments, the SAP protein comprises an SAP protein with glycosylation that differs from that of human SAP purified from serum, and the additional anticancer therapeutic is a JAK kinase inhibitor. In certain embodiments, the hemoglobin levels are increased by about 500-1000 mg/L, 1-2 g/L, 2-3 g/L, or 3-5 g/L. In certain embodiments, the hemoglobin levels are increased to at least about 80 g/L, 90 g/L, 100 g/L, 110 g/L, 120 g/L, 130 g/L, or 140 g/L. In some embodiments, the increase in hemoglobin levels is seen for ≥12 consecutive weeks following treatment. In some embodiments, the hemoglobin levels are increased by about ≥10 g/L for ≥12 consecutive weeks following treatment (e.g., greater than 24 weeks, greater than 30 weeks, greater than 36 weeks, greater than 42 weeks, greater than 48 weeks) without transfusions. In some embodiments, the hemoglobin levels are increased by about ≥20 g/L is seen for ≥12 consecutive weeks following treatment (e.g., greater than 24 weeks, greater than 30 weeks, greater than 36 weeks, greater than 42 weeks, greater than 48 weeks) without transfusions. In some embodiments, the increase in hemoglobin levels to at least about 100 g/L is seen for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in reducing red blood cell (RBC) transfusions by at least 20%, at least 25%, at least 30%, at least 35%, at least 40%, at least 45%, at least 50%, at least 55%, or at least 60% compared to the level prior to commencing treatment with the methods provided herein. In some embodiments, the treatment is effective in reducing RBC transfusions by at least 25%. In some embodiments, the treatment is effective in reducing RBC transfusions by at least 50%. In some embodiments, the treatment is effective in achieving RBC transfusion independence. In certain embodiments, the disclosure provides methods for reducing RBC transfusions in a patient in need thereof, wherein the patient in need thereof has myelofibrosis, comprising administering an amount of an SAP protein, according to a dosing schedule effective to reduce RBC transfusions by at least about 25%, at least 30%, at least 35%, at least 40%, at least 50%, at least 55%, or at least 60%. In certain embodiments, the SAP protein comprises an SAP protein with glycosylation that differs from that of human SAP purified from serum, and the additional anticancer therapeutic is a JAK kinase inhibitor. In certain embodiments, RBC transfusions are reduced by about 25-60%, by about 25-55%, or by about 25-50%. In certain embodiments, the patient becomes transfusion independent following treatment. In certain embodiments, the patient becomes transfusion independent for ≥12 consecutive weeks following treatment. In certain embodiments, the patient has a ≥50% reduction in transfusions for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in ameliorating thrombocytopenia when present. In some embodiments, the treatment increases platelets by at least 50%, at least 60%, at least 70%, at least 80%, at least 90%, or at least 100% compared to the level prior to commencing treatment with the methods provided herein. In some embodiments, the treatment increases platelets by at least 20%-30%, at least 30%-40%, at least 40%-50%, at least 50%-60%, at least 60%-70%, at least 70%-80%, at least 80%-90%, or at least 90%-100% compared to the level prior to commencing treatment with the methods provided herein. In some embodiments, the treatment is effective in increasing platelets by at least 100%. In some embodiments, the treatment increases platelets to at least 25×10⁹/L, 30×10⁹/L, 40×10⁹/L, 50×10⁹/L, 60×10⁹/L, 70×10⁹/L, 80×10⁹/L, 90×10⁹/L, or 100×10⁹/L. In some embodiments, the treatment increases platelets to at least 25-50×10⁹/L, 50-75×10⁹/L, 75-100×10⁹/L, or 100-150×10⁹/L. In some embodiments, the treatment increases platelets to 50×10⁹/L. In some embodiments, the treatment increases platelets to 100×10⁹/L. In some embodiments, platelets are measured as part of a routine Complete Blood Count (CBC). It would be understood by one of skill in the art that other known methods to measure platelets may also be employed. In certain embodiments, the disclosure provides methods for increasing platelets in a patient in need thereof, wherein the patient in need thereof has myelofibrosis, comprising administering an amount of an SAP protein, according to a dosing schedule effective to increase platelets by at least 25%, at least 30%, at least 40%, at least 50%, at least 60%, at least 70%, at least 80%, at least 90%, or at least 100%. In certain embodiments, the SAP protein comprises an SAP protein with glycosylation that differs from that of human SAP purified from serum, and the additional anticancer therapeutic is a JAK kinase inhibitor. In certain embodiments, platelets are increased by about 50%-60%, 60%-70%, 70%-80%, 80%-90%, or 90%-100%. In certain embodiments, the patient has a platelet count >25×10⁹/L for ≥12 consecutive weeks following treatment. In certain embodiments, the patient has a platelet count >50×10⁹/L for ≥12 consecutive weeks following treatment. In certain embodiments, the patient has a platelet count ≥100×10⁹/L for ≥12 consecutive weeks following treatment. In certain embodiments, the patient has a doubling of baseline platelet count for ≥12 consecutive weeks without transfusions. In certain embodiments, the patient has a ≥50% reduction in transfusions for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in improving either or both of hemoglobin levels or platelet levels in subjects with both hemoglobin <100 g/L and platelets <50×10⁹/L, with no worsening of hemoglobin or platelets from baseline. In some embodiments, the treatment using the methods described herein (e.g. single agent or combination therapy using an SAP protein) is effective in improving hemoglobin levels in subjects with hemoglobin <100 g/L, with no worsening of platelets to <50×10⁹/L. In some embodiments, the treatment using the methods described herein (e.g. single agent or combination therapy using an SAP protein) is effective in improving platelet levels in subjects with platelets to <50×10⁹/L, with no worsening of hemoglobin to <100 g/L or new transfusion dependence.

In certain embodiments, the treatment using the methods described herein (e.g., single agent or combination therapy using SAP) increases progression-free survival and/or overall survival, such as versus the standard of care. In certain embodiments, progression-free survival and/or overall survival is measured versus no treatment, or versus a standard of care therapy.

In some embodiments, the treatment using the methods described herein (e.g. single agent or combination therapy using an SAP protein) is effective at decreasing platelet transfusions by at least 25%, 30%, 40%, 50%, 60%, 75%, or 100% compared to the level prior to commencing treatment with the methods provided herein. In some embodiments, the treatment decreases platelet transfusions by at least 50%. In certain embodiments, the disclosure provides methods for decreasing platelet transfusions in a patient in need thereof, wherein the patient in need thereof has myelofibrosis, comprising administering an amount of an SAP protein, according to a dosing schedule effective to decrease platelet transfusions by at least 30%, at least 40%, at least 50%, at least 60%, or at least 70%. In certain embodiments, the SAP protein comprises an SAP protein with glycosylation that differs from that of human SAP purified from serum. In certain embodiments, platelet transfusions are decreased by about 25%-40%, 25%-50%, 50%-70%, or 700%-100%. In certain embodiments, the patient becomes transfusion independent for ≥12 consecutive weeks following treatment. In certain embodiments, the patient has a ≥50% reduction in transfusions for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in ameliorating thrombocytosis when present. In some embodiments, the treatment decreases platelets by at least 10%, at least 15%, at least 20%, at least 25%, at least 30%, at least 35%, at least 40%, or at least 50% compared to the level prior to commencing treatment with the methods provided herein. In some embodiments, the treatment decreases platelets by 25%. In some embodiment, the treatment decreases platelets to the normal levels. In some embodiments, platelets are measured as part of a routine Complete Blood Count (CBC). It would be understood by one of skill in the art that other known methods to measure platelets may also be employed. In certain embodiments, the disclosure provides methods for decreasing platelets in a patient in need thereof, wherein the patient in need thereof has myelofibrosis, comprising administering an amount of an SAP protein, according to a dosing schedule effective to decrease platelets by at least 10%, at least 15%, at least 20%, at least 25%, at least 30%, at least 35%, at least 40%, or at least 50%. In certain embodiments, the SAP protein comprises an SAP protein with glycosylation that differs from that of human SAP purified from serum. In certain embodiments, platelets are decreased by about 10%-15%, at least 15%-25%, or at least 25%-35%. In certain embodiments, the reduction in platelet count is seen for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in ameliorating neutropenia when present. In some embodiments, the treatment increases the absolute neutrophil count (ANC) by at least 25%, at least 30%, at least 40%, at least 50%, at least 60%, at least 70%, at least 80%, at least 90%, or at least 100% compared to the level prior to commencing treatment with the methods provided herein. In some embodiments, the treatment increases ANC by at least 20%-30%, at least 30%-40%, at least 40%-50%, at least 50%-60%, at least 60%-70%, at least 70%-80%, at least 80%-90%, or at least 90%-100% compared to the level prior to commencing treatment with the methods provided herein. In some embodiments, the treatment is effective in increasing ANC by at least 50%. In some embodiments, the treatment increases ANC to at least 1000 cells/μL, at least 1250 cells/μL, at least 1500 cells/μL, at least 1750 cells/μL, or at least 2000 cells/μL. In some embodiments, the treatment increases ANC to at least 1250-1500 cells/μL, at least 1500-1750 cells/μL, or at least 1750-2000 cells/μL. In some embodiments, the treatment increases ANC to at least 1500 cells/μL. In some embodiments, ANC is measured as part of a routine Complete Blood Count (CBC). It would be understood by one of skill in the art that other known methods to measure ANC may also be employed. In certain embodiments, the disclosure provides methods for increasing ANC in a patient in need thereof, wherein the patient in need thereof has myelofibrosis, comprising administering an amount of an SAP protein, according to a dosing schedule effective to increase ANC by at least 25%6, at least 30%, at least 40%, at least 50%, at least 60%, at least 70%, at least 80%, at least 90%, or at least 100%. In certain embodiments, SAP protein comprises an SAP protein with glycosylation that differs from that of human SAP purified from serum. In certain embodiments, ANC is increased by about 20%-30%, at least 30%-40%, at least 40%-50%, at least 50%0-60%0, at least 60%-706, at least 70%-80%, at least 80%-90% or at least 90%-100%. In some embodiments, the treatment increases ANC to at least 1500 cells/μL for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in ameliorating leukopenia when present. In some embodiments, the treatment increases the white blood cells (WBC) by at least 25%, at least 30%, at least 40%, at least 50%, at least 60%, at least 70%, at least 80%, at least 90%, or at least 100% compared to the level prior to commencing treatment with the methods provided herein. In some embodiments, the treatment increases WBC by at least 20%-30%, at least 30%-40%, at least 40%-50%, at least 50%-60%, at least 60%-70%, at least 70%-80%, at least 80%-90%, or at least 90%-100% compared to the level prior to commencing treatment with the methods provided herein. In some embodiments, the treatment is effective in increasing WBC by at least 50%. In some embodiments, the treatment increases WBC to at least 4×10⁹/L, 5×10⁹/L, 7.5×10⁹/L, or 10×10⁹/L. In some embodiments, the treatment increases WBC to 10×10⁹/L. In some embodiments, the treatment increases WBC to the normal range. In some embodiments, WBC is measured as part of a routine Complete Blood Count (CBC). It would be understood by one of skill in the art that other known methods to measure WBC may also be employed. In certain embodiments, the disclosure provides methods for increasing WBC in a patient in need thereof, wherein the patient in need thereof has myelofibrosis, comprising administering an amount of an SAP protein, according to a dosing schedule effective to increase WBC by at least 25%, at least 30%, at least 40%, at least 50%, at least 60%, at least 70%, at least 80%, at least 90%, or at least 100%. In certain embodiments, the SAP protein comprises an SAP protein with glycosylation that differs from that of human SAP purified from serum. In certain embodiments, WBC is increased by about 20%-30%, at least 30%-40%, at least 40%-50%, at least 50%-60%, at least 60%-70%, at least 70%-80%, at least 80%-90%, or at least 90%-100%. In some embodiments, the increase in WBC is seen for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in ameliorating leukocytosis when present. In some embodiments, the treatment decreases ANC by at least 10%, at least 15%, at least 20%, at least 25%, at least 30%, at least 35%, at least 40%, at least 50%, at least 60%, or at least 70% compared to the level prior to commencing treatment with the methods provided herein, without decreasing ANC below 1500/μL. In some embodiments, the treatment decreases ANC by 25%. In some embodiments, the treatment decreases ANC by 50%. In some embodiments, the treatment decreases ANC to normal levels. In some embodiments, the treatment decreases white blood cells (WBC) by at least 10%, at least 15%, at least 20%, at least 25%, at least 30%, at least 35%, at least 40%, at least 50%, at least 60%, or at least 70% compared to the level prior to commencing treatment with the methods provided herein, without decreasing WBC below the lower limit of normal. In some embodiments, the treatment decreases WBC by 25%. In some embodiments, the treatment decreases WBC by 50%. In some embodiments, the treatment decreases WBC to <35×10⁹/L, <30×10⁹/L, <25×10/L, <20×10⁹/L, or <15×10⁹/L. In some embodiments, the treatment decreases WBC to <25×10⁹/L. In some embodiments, the treatment decreases WBC to the normal range. In some embodiments, ANC and WBC are measured as part of a routine Complete Blood Count (CBC). It would be understood by one of skill in the art that other known methods to measure ANC or WBC may also be employed. In certain embodiments, the disclosure provides methods for decreasing ANC or WBC in a patient in need thereof, wherein the patient in need thereof has myelofibrosis, comprising administering an amount of an SAP protein, according to a dosing schedule effective to decrease ANC or WBC by at least 10%, at least 15%, at least 20%, at least 25%, at least 30%, at least 35%, at least 40%, at least 50%, at least 60%, or at least 70% without decreasing WBC below the lower limit of normal. In certain embodiments, the SAP protein comprises an SAP protein with glycosylation that differs from that of human SAP purified from serum. In certain embodiments, ANC or WBC is decreased by about 20%-30%, at least 30%-40%, at least 40%-50%, at least 50%-60%, or at least 60%-70% without decreasing WBC below the lower limit of normal. In some embodiments, the treatment decreases WBC to <25×10⁹/L for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in decreasing peripheral blood blasts by at least 20%, at least 25%, at least 30%, at least 35%, at least 40%, at least 45%, at least 50%, at least 55%, at least 60%, or at least 70% compared to the level prior to commencing treatment with the methods provided herein. In some embodiments, the treatment is effective in decreasing peripheral blood blasts by at least 50%. In some embodiments, the treatment is effective in decreasing peripheral blood blasts from ≥1% to <1%. It would be understood by one of skill in the art that any of the methods known in the art to measure peripheral blood blasts may be employed. In certain embodiments, the disclosure provides methods for decreasing peripheral blood blasts in a patient in need thereof, wherein the patient in need thereof has myelofibrosis, comprising administering an amount of an SAP protein, according to a dosing schedule effective to decrease peripheral blood blasts by at least 20%, at least 25%, at least 30%, at least 35%, at least 40%, at least 45%, at least 50%, at least 55%, at least 60%, or at least 70%. In certain embodiments, the SAP protein comprises an SAP protein with glycosylation that differs from that of human SAP purified from serum. In certain embodiments, peripheral blood blasts are decreased by about 20%-30%, at least 30%-40%, at least 40%-50%, at least 50%-60%, or at least 60%-70%. In certain embodiments, peripheral blood blasts are decreased from ≥1% to <1%. In certain embodiments, peripheral blood blasts are decreased from ≥1% to <1% for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in decreasing bone marrow fibrosis from Grade 3 to Grade 2. In some embodiments, the treatment is effective in decreasing bone marrow fibrosis from Grade 3 to Grade 1. In some embodiments, the treatment is effective in decreasing bone marrow fibrosis from Grade 3 to Grade 0. In some embodiments, the treatment is effective in decreasing bone marrow fibrosis from Grade 2 to Grade 1. In some embodiments, the treatment is effective in decreasing bone marrow fibrosis from Grade 2 to Grade 0. In some embodiments, the treatment is effective in decreasing bone marrow fibrosis by at least by 5, 10, 20, 30, 40, 50, 60, 70, 80, or 90% compared to the level prior to commencing treatment with the methods provided herein. It would be understood by one of skill in the art that any of the methods known in the art to evaluate bone marrow fibrosis may be employed. In certain embodiments, the disclosure provides methods for decreasing bone marrow fibrosis in a patient in need thereof, wherein the patient in need thereof has myelofibrosis, comprising administering an amount of an SAP protein, according to a dosing schedule effective to decrease bone marrow fibrosis by at least 5, 10, 20, 30, 40, 50, 60, 70, 80, or 90%1′. In certain embodiments, the SAP protein comprises an SAP protein with glycosylation that differs from that of human SAP purified from serum. In certain embodiments, bone marrow fibrosis is decreased by about 20%-30%, at least 30%-40%, at least 40%-50%, at least 50%-60%, or at least 60%-70%. In certain embodiments, the decrease in bone marrow fibrosis by greater than 1 grade is seen for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in decreasing bone marrow fibrosis as measured by quantitative image analysis. In some embodiments, the treatment is effective in decreasing bone marrow fibrosis by at least by 5, 10, 20, 30, 40, 50, 60, 70, 80, or 90% compared to the level prior to commencing treatment with the methods provided herein. It would be understood by one of skill in the art that any of quantitative image analysis methods known in the art to evaluate bone marrow fibrosis may be employed. In some embodiments, computer assisted image analysis (CIA) is performed on whole slide scans from serial bone marrow specimens from a patient for objective quantification of overall fibrosis level and osteoschlerosis of all post-treatment samples compared to baseline samples. The areas occupied with bone trabeculae (% of total core biopsy) and reticulin fibers (% of hematopoietic areas excluding the fat) are calculated. In certain embodiments, the disclosure provides methods for decreasing bone marrow fibrosis in a patient in need thereof, wherein the patient in need thereof has myelofibrosis, comprising administering an amount of an SAP protein, according to a dosing schedule effective to decrease bone marrow fibrosis by at least 5, 10, 20, 30, 40, 50, 60, 70, 80, or 90%. In certain embodiments, the SAP protein comprises an SAP protein with glycosylation that differs from that of human SAP purified from serum. In certain embodiments, bone marrow fibrosis is decreased by about 20%-30%, at least 30%-40%, at least 40%-50%, at least 50%-60%, or at least 60%-70%. In certain embodiments, a decrease in bone marrow fibrosis of greater than 10% is seen for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in improving bone marrow morphology indicative of healing and restoration of hematopoiesis. In some embodiments, the treatment is effective in improving megakaryocytic topography by at least 5, 10, 20, 30, 40, 50, 60, 70, 80, or 90% compared to the level prior to commencing treatment with the methods provided herein. In some embodiments, the treatment is effective in normalizing the myeloid to erythroid (M:E) ratio by at least 5, 10, 20, 30, 40, 50, 60, 70, 80, or 90% compared to the level prior to commencing treatment with the methods provided herein. In some embodiments, the treatment is effective in reducing collagen and osteosclerosis by at least 5, 10, 20, 30, 40, 50, 60, 70, 80, or 90% compared to the level prior to commencing treatment with the methods provided herein. It would be understood by one of skill in the art that any of the methods known in the art to evaluate bone marrow morphology may be employed. In certain embodiments, the disclosure provides methods for improving bone marrow morphology (e.g., one or more of improving megakaryocytic topography, normalizing M:E ratio, reducing collagen and osteosclerosis) in a patient in need thereof, wherein the patient in need thereof has myelofibrosis, comprising administering an amount of an SAP protein, according to a dosing schedule effective to improve bone marrow morphology by at least 5, 10, 20, 30, 40, 50, 60, 70, 80, or 90%. In certain embodiments, the SAP protein comprises an SAP protein with glycosylation that differs from that of human SAP purified from serum. In certain embodiments, bone marrow morphology is improved by about 20%-30%, at least 30%-40%, at least 40%-50%, at least 50%-60%, or at least 60%-70%. In certain embodiments, an improvement in bone marrow morphology of greater than 10% is seen for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in decreasing fibrosis in bone marrow, spleen, and liver. In some embodiments, the fibrosis is measured by Positron Emission Tomography/Computed Tomography (PET/CT). In some embodiments, the fibrosis is measured by 3′-¹⁸Fluoro-3′-deoxy-L-thymidine (¹⁸F-FLT) PET (Andreoli et al. ASH 2014 Abstract 3195). In some embodiments, the fibrosis is measured by 1866 ¹⁸F-Fluorodeoxyglucose Positron Emission Tomography/Computed Tomography ¹⁸FDG-PET/CT (Derlin et al. ASH 2014 Abstract 1866). In some embodiments, the treatment is effective in decreasing bone marrow, spleen, or liver fibrosis by at least by 5, 10, 20, 30, 40, 50, 60, 70, 80, or 90% compared to the level prior to commencing treatment with the methods provided herein. In certain embodiments, the disclosure provides methods for decreasing bone marrow, spleen, or liver fibrosis in a patient in need thereof, wherein the patient in need thereof has myelofibrosis, comprising administering an amount of an SAP protein, according to a dosing schedule effective to decrease bone marrow, spleen, or liver fibrosis by at least 5, 10, 20, 30, 40, 50, 60, 70, 80, or 90%. In certain embodiments, the SAP protein comprises an SAP protein with glycosylation that differs from that of human SAP purified from serum. In certain embodiments, bone marrow, spleen, or liver fibrosis is decreased by about 20%-30%, at least 30%-40%, at least 40%-50%, at least 50%-60%, or at least 60%-70%. In certain embodiments, the decrease in bone marrow, spleen, or liver fibrosis by greater than 10% is seen for ≥12 consecutive weeks following treatment (e.g., greater than 24 weeks, greater than 30 weeks, greater than 36 weeks, greater than 42 weeks, greater than 48 weeks).

In some embodiments, the treatment using the methods described herein (e.g. single agent or combination therapy using an SAP protein) is effective in decreasing bone marrow blasts from ≥5% to <5%. It would be understood by one of skill in the art that any of the methods known in the art to measure bone marrow blasts be employed. In certain embodiments, the disclosure provides methods for decreasing bone marrow blasts in a patient in need thereof, wherein the patient in need thereof has myelofibrosis, comprising administering an amount of an SAP protein according to a dosing schedule effective to decrease bone marrow blasts by at least 20%, at least 25%, at least 30%, at least 35%, at least 40%, at least 45%, at least 50%, at least 55%, at least 60%, or at least 70%. In certain embodiments, the SAP protein comprises an SAP protein with glycosylation that differs from that of human SAP purified from serum. In certain embodiments, bone marrow blasts are decreased by about 20%-30%, at least 30%-40%, at least 40%0-50%, at least 50%00-606, or at least 60%-70%. In certain embodiments, the decrease in bone marrow blasts is seen for ≥12 consecutive weeks following treatment (e.g., greater than 24 weeks, greater than 30 weeks, greater than 36 weeks, greater than 42 weeks, greater than 48 weeks).

In some embodiments, the treatment is effective in improving bone marrow cellularity. The improvement can be at least by 20, 30, 40, 50, 60, or 70% compared to the level prior to commencing treatment with the methods provided herein. In certain embodiments, the disclosure provides methods for improving bone marrow cellularity in a patient in need thereof, wherein the patient in need thereof has myelofibrosis, comprising administering an amount of an SAP protein according to a dosing schedule effective to improve bone marrow cellularity by at least 20%, at least 25%, at least 30%, at least 35%, at least 40%, at least 45%, at least 50%, at least 55%, at least 60%, or at least 70%. In certain embodiments, the SAP protein comprises an SAP protein with glycosylation that differs from that of human SAP purified from serum. In certain embodiments, bone marrow cellularity is improved by about 20%-30%, at least 30%-40%, at least 40%-50%, at least 500-60%, or at least 60%-70%. In certain embodiments, the improvement in bone marrow cellularity is seen for ≥12 consecutive weeks following treatment (e.g., greater than 24 weeks, greater than 30 weeks, greater than 36 weeks, greater than 42 weeks, greater than 48 weeks).

In some embodiments, the treatment is effective in decreasing leukoerythroblastosis. In some embodiments, the treatment is effective in eliminating leukoerythroblastosis. In some embodiments, the treatment is effective in decreasing or eliminating leukoerythroblastosis for ≥12 consecutive weeks following treatment. In certain embodiments, the treatment using the methods described herein (e.g. single agent or combination therapy using an SAP protein) results in at least one of the effects described herein (e.g. reduction in mutant allele burden, reduction in spleen volume, reduction in MPN-SAF Total Symptom Score, improving quality of life as measured by the EORTC QLQ-C30, increase in hemoglobin, reduction in RBC transfusions, improvement in thrombocytopenia, decrease in platelet transfusions, improvement in thrombocytosis, improvement in neutropenia, improvement in leukocytosis, decrease in peripheral blood blasts, decrease in bone marrow fibrosis, decrease in bone marrow blasts, decrease in peripheral blood blasts, or improvement in bone marrow cellularity). In some embodiments, the treatment using the methods described herein results in at least two of the effects described herein. In some embodiments, the treatment using the methods described above results in at least three, four, five, six, seven, eight, nine, or ten of the effects described herein. In certain embodiments of any of the foregoing, evaluation of whether a particular degree of improvement of a symptom or therapeutic effect has been achieved is evaluated at one or more points over time, such as following at least 12, 18, 20, or at least 24 weeks of treatment, or following greater than 24 weeks of treatment (e.g., greater than 30 weeks, greater than 36 weeks, greater than 42 weeks, greater than 48 weeks).

In some embodiments, treatment using one or more of the methods described herein (e.g. single agent or combination therapy using an SAP protein of the disclosure) results in at least one of the effects described herein (e.g. reduction in spleen volume, reduction in MPN-SAF Total Symptom Score, improving quality of life as measured by the EORTC QLQ-C30, increase in hemoglobin, reduction in RBC transfusions, achievement of transfusion independence, improvement in thrombocytopenia, decrease in platelet transfusions, improvement in thrombocytosis, improvement in neutropenia, improvement in leukocytosis, decrease in peripheral blood blasts, decrease in bone marrow fibrosis, decrease in bone marrow blasts or improvement in bone marrow cellularity), without causing or inducing clinically significant myelosuppression. In some embodiments, treatment using one or more of the methods described herein results in at least two of the effects described herein, without causing or inducing clinically significant myelosuppression. In some embodiments, treatment using one or more of the methods described above results in at least three, four, five, six, seven, eight, nine, or ten of the effects described herein, without causing or inducing clinically significant myelosuppression. In some embodiments, treatment using one or more of the methods described herein results in no myelosuppression. It certain embodiments, any of the foregoing methods comprise administering SAP comprising an SAP protein having glycosylation that differs from that of SAP purified from human serum, such as recombinant human SAP (e.g., recombinant human pentraxin-2 produced in CHO cells). In certain embodiments, any of the foregoing methods comprise administering the SAP protein according to a dosing schedule, wherein any of the foregoing therapeutic effects are achieved following administration according to the dosing schedule. In certain embodiments, one or more of the foregoing therapeutic effects are achieved following administration according to a dosing schedule (e.g., administering comprises administering according to a dosing schedule). Improvement in any of the foregoing parameters (e.g., reduction in symptoms) is evaluated at one or more time points during treatment, for example, following at least 12, at least 18, at least 20, at least 24, or greater than 24 weeks of treatment (e.g., greater than 30 weeks, greater than 36 weeks, greater than 42 weeks, greater than 48 weeks).

For any of the foregoing examples of improvement in a patient, such as an improvement in one or more symptoms, in certain embodiments, the disclosure provides that the treatment comprises administering an SAP protein at a dose and on a dosing schedule effective to have the therapeutic effect. In certain embodiments, such as certain embodiments of any of the foregoing, SAP is administered without an additional anti-cancer therapeutic.

In some embodiments, the SAP protein is administered at a dosing schedule comprising administration of the SAP protein every 4 weeks for at least 1 cycle, at least 2 cycles, at least 3 cycles, at least 4 cycles, at least 5 cycles, at least 6 cycles, at least 7 cycles or at least 8 cycles of a 28-day or 4-week cycle. In some embodiments, the SAP protein is administered to the subject once every 4 weeks for at least 6 cycles of a 28-day cycle, at least 8 cycles of a 28-day cycle, at least 10 cycles of a 28-day cycle, at least 12 cycles of a 28-day cycle, at least 15 cycles of a 28-day cycle, at least 18 cycles of a 28-day cycle, or at least 24 cycles of a 28-day cycle. In some embodiments, the compound is administered to the subject once every 4 weeks for at least one month, at least two months, at least three months, at least four months, at least five months, at least six months, at least eight months, at least one year, or at least two years, and possibly administered chronically over the life of the patient. In a further embodiment, the SAP protein is administered every other day in the first week of treatment. In some embodiments, the SAP protein is administered several days (e.g. days 1, 3 and 5) every 4 weeks for at least 6 cycles of a 28-day cycle, at least 8 cycles of a 28-day cycle, at least 10 cycles of a 28-day cycle, at least 12 cycles of a 28-day cycle, at least 15 cycles of a 28-day cycle, at least 18 cycles of a 28-day cycle, or at least 24 cycles of a 28-day cycle. In some embodiments, the compound is administered to the subject for several days (e.g., days 1, 3, 5) every 4 weeks for at least one month, at least two months, at least three months, at least four months, at least five months, at least six months, at least eight months, at least one year, or at least two years, and possibly administered chronically over the life of the patient. In some embodiments, the SAP protein is administered to the subject at a dosing schedule comprising administration of the SAP protein at least once a week for at least 1 cycle, at least 2 cycles, at least 3 cycles, at least 4 cycles, at least 5 cycles, at least 6 cycles, at least 7 cycles or at least 8 cycles of a 28-day cycle. In some embodiments, the SAP protein is administered to the subject at least once a week for at least 6 cycles of a 28-day cycle, at least 8 cycles of a 28-day cycle, at least 10 cycles of a 28-day cycle, at least 12 cycles of a 28-day cycle, at least 15 cycles of a 28-day cycle, at least 18 cycles of a 28-day cycle, or at least 24 cycles of a 28-day cycle. In some embodiments, the compound is administered to the subject once a week for at least one month, at least two months, at least three months, at least four months, at least five months, at least six months, at least eight months, at least one year, or at least two years. In further embodiments, the compound is administered every other day in the first week of treatment. In certain embodiments, the dosing schedule results in at least one of the effects (e.g., improvement in one or more symptoms or parameters) described herein (e.g. reduction in spleen volume, reduction in MPN-SAF Total Symptom Score, increase in hemoglobin, reduction in RBC transfusions, achievement of transfusion independence, improvement in thrombocytopenia, decrease in platelet transfusions, improvement in thrombocytosis, improvement in neutropenia, improvement in leukocytosis, decrease in peripheral blood blasts, decrease in bone marrow fibrosis, decrease in bone marrow blasts or improvement in bone marrow cellularity). In some embodiments, the dosing schedule results in at least two of the effects described herein. In some embodiments, the dosing schedule results in at least three, four, five, six, seven, eight, nine, or ten of the effects described herein. In certain embodiments, the SAP agonist comprises recombinant human SAP.

In certain embodiments, the disclosure provides methods for administering an amount of an SAP protein, according to a dosing schedule comprising administering an SAP protein using a dosage regimen comprising administering 10 mg/kg of an SAP protein, such as an SAP protein with glycoslation that differs from that of human SAP purified from serum, on days 1, 3, 5 of Cycle 1 and Day 1 each subsequent 28 day cycle.

In certain embodiments, the disclosure provides methods for administering an amount of an SAP protein, according to a dosing schedule comprising administering an SAP protein using a dosage regimen comprising administering 3 mg/kg of an SAP protein on Days 1, 3, and 5 of Cycle 1 and Day 1 of each subsequent 28 day cycle.

In certain embodiments, the disclosure provides methods for administering an amount of an SAP protein, according to a dosing schedule comprising administering an SAP protein using a dosage regimen comprising administering 0.3 mg/kg of an SAP protein on Days 1, 3, and 5 of Cycle 1 and Day 1 of each subsequent 28 day cycle.

In some embodiments, the SAP protein is administered multiple times during the first week (e.g., days 1, 3 and 5), followed by administration every week, every two weeks, every three weeks, or every 4 weeks. In some embodiments, the SAP protein is administered multiple times a week every other week, every three weeks, every month, every other month, every three months, every six months, or as needed. In some embodiments, the SAP protein is administered by IV infusion. In some embodiments, the SAP protein is administered subcutaneously. In some embodiments, the SAP protein is administered at a dose of 10 mg/kg. In some embodiments, the SAP protein is administered at a dose of 3 mg/kg. In some embodiments, the SAP protein is administered at a dose of 0.3 mg/kg. In some embodiments, the SAP protein is administered at any of the dosages described herein. In some embodiments, the dosage regimens described herein are adjusted as needed to achieve one of the treatment outcomes described herein.

In some embodiments, the methods disclosed herein comprise administering one or more additional doses of the SAP protein after achieving an initial response. In some embodiments, a subsequent response is achieved following the administration of one or more additional doses of the SAP protein after achieving an initial response in a subject. A subsequent response may be an additional response (e.g. any of the responses described herein not initially observed), the maintenance of the initial response, or an improvement upon the initial response. In some embodiments, the administration of one or more additional doses substantially maintains the initial response. In some embodiments, the administration of one or more additional doses provides further improvement relative to the initial response. In some embodiments, the administration of one or more additional doses provides one or more additional responses that were not initially observed. In certain embodiments, the SAP protein comprises recombinant human SAP.

In some embodiments, upon administration of an SAP protein or a pharmaceutically acceptable salt thereof to a subject such as human subject, the Cₘₐₓ (maximum drug concentration) of the compound is achieved within about 0.5 to about 5 hours, about 1.5 to about 4.5 hours, about 2 to about 4 hours, or about 2.5 to about 3.5 hours post-dose. In some embodiments, upon administration of the compound to a human subject, the elimination half life of the compound is about 11 to 110 hours, 20-72 hours, 12 to about 40 hours, about 16 to about 34 hours, or about 20 to about 40 hours. In some embodiments, the mean AUC of the compound increases more than proportionally with increasing doses ranging from about 0.1 mg to about 40 mg per kg. In some embodiments, the accumulation of the compound is about 1.1 to about 5 fold, about 1.25 to about 4.0 fold, about 1.5 to about 3.5 fold, about 2 to about 3 fold at steady state when the compound is dosed once weekly. In some embodiments, the compound does not accumulate when dosed weekly.

### SAP Proteins and SAP Agonists

One aspect of the disclosure provides SAP proteins useful in the treatment of myeloproliferative disorders. One aspect of the disclosure provides SAP agonists useful in the treatment of myeloproliferative disorders. SAP agonists encompass all compounds and compositions that increase or otherwise mimic endogenous SAP signaling, including compounds that increase SAP activity. In certain embodiments of the any of the foregoing methods, an SAP agonist can be used wherever an SAP protein is being used, alone or in combination with an SAP protein of the disclosure. Exemplary SAP agonists are described below. Throughout the disclosure, “SAP proteins” are referred to. Unless otherwise specified, such reference contemplates the use of any of the SAP proteins disclosed herein, including use of recombinant SAP, such as SAP protein comprising human SAP, which SAP protein has a glycosylation that differs from that of SAP isolated from human serum. The disclosure contemplates use of any of the SAP proteins and SAP agonists disclosed herein in any of the methods described herein, including use alone or as a combination therapy.

### SAP

SAP or pentraxin-2 is a naturally occurring serum protein in mammals composed of five identical subunits, or protomers, which are non-covalently associated in a disk-like complex. SAP belongs to the pentraxin superfamily of proteins, which are characterized by this cyclic pentameric structure. The classical short pentraxins include SAP as well as C-reactive protein (Osmand, A. P., et al., Proc. Nat. Acad. Sci., 74: 739-743, 1997). The long pentraxins include pentraxin-3. SAP is normally synthesized in the liver and has a physiological half-life of twenty-four hours. Human SAP (hSAP) circulates at approximately 20-40 μg/ml in plasma as a homopentamer. The sequence of the human SAP subunit is disclosed in SEQ ID NO: 1, which corresponds to amino acids 20-223 of Genbank Accession NO. NP_001630 (signal sequence not depicted).

Previous research demonstrates that SAP has an important role in both the initiation and resolution phases of the immune response. hSAP functions in innate resistance to microbes and in the scavenging and phagocytosis of cellular debris and appears to play a role in regulation of wound healing and fibrosis. These functions may involve (i) binding to ligands associated with microbes and cellular debris, as specified above, and various extracellular matrix proteins in a Ca²⁺-dependent manner, (ii) binding to C1q for complement activation by promoting opsonization by C3b and iC3b, (iii) binding to Fcγ receptors to initiate direct opsonization and subsequent phagocytosis or endocytosis, and (iv) subsequent regulation of monocyte function and differentiation. Accordingly, hSAP molecules localize to sites of injury and repair and may target and/or concentrate in these locations through binding these molecules.

The 3D structure of hSAP has been determined by X-ray crystallography and several crystal structures complexed with different ligands have also been reported. The pentameric structure of hSAP has 5-fold rotational symmetry and is fairly rigid with a pore. The diameter of the hSAP pentamer is approximately 100 Å, and the central pore is 20 Å in diameter and 35 Å deep. Each protomer is constructed of antiparallel β-strands arranged in two sheets, with a hydrophobic core with a jellyroll topology. The hSAP pentamer has 2 faces, an A-face, which possesses five α helices, one on each protomer, and a B face with 5 sets of double calcium-binding sites. The B-face is thought to provide a calcium-dependent ligand binding face, and several calcium-dependent ligands that bind the B-face have been identified, including phosphorylethanolamine, DNA, heparan sulfate, dermatan sulfate and dextran sulfate, laminin and collagen IV. The A-face of hSAP also appears to bind molecules such as C1q and may mediate phagocytosis through binding to Fey receptors. Each protomer may be glycosylated at Asn32, a single site.

N- and C-termini are solvent accessible and are located on the inner edge of each protomer molecule. The N-terminus is located on the outer edge of each protomer and on the perimeter of the ring formed by the 5 protomers. The C-terminus is located more toward the inner perimeter and pore of the pentamer ring but is directed outward toward the A face. N- and C-termini within one protomer are about 25 Å apart. The termini do not appear to be involved in subunit interactions and they are away from the glycan chain attached at Asn32. The subunits of hSAP are held together non-covalently with approximately 15% of the surface of each subunit involved in these interactions. These extensive interactions account for the considerable stability of the hSAP pentamer.

The SAP encompassed by embodiments described herein includes SAP from any source such as, for example, human SAP or isomers or analogs from other vertebrate or mammalian sources. SAP further encompasses SAP molecules having modifications from the native PTX-2 amino acid sequence introduced by, for example, site-directed mutagenesis. Such modification may alter specific amino acids and/or other features of the molecule, while retaining the general pentameric pentraxin nature of the molecule. The “SAP protein” may be used to encompass both SAP pentamers and SAP protomers. “SAP pentamer” or “pentameric SAP” refers to a protein complex at least including five SAP protomers, and “SAP protomer” refers to one individual protein unit of the SAP pentamer. In certain embodiments of any of the aspects and embodiments of the disclosure, the disclosure comprises administration of an SAP protein comprising one or more protomers comprising the amino acid sequence set forth in SEQ ID NO: 1. In certain embodiments, the SAP protein is a protein comprising five protomers. In certain embodiments, the SAP protein comprises recombinant human SAP. An exemplary recombinant human SAP comprises PRM-151. In certain embodiments, the SAP agonist comprises recombinant SAP. Methods of making proteins generally, and human pentraxin-2 specifically, recombinantly are known in the art. Suitable cells for recombinant expression, such as insect or mammalian cells may be selected. SEQ ID NO: 1 corresponds to the amino acid sequence of a human SAP protomer.

Modification of a glycan structure on a human SAP protein can increase the biological activity of the SAP protein relative to a corresponding sample of wild-type SAP isolated from human serum. Isolated SAP from human serum contains only α2,6-linked sialic acid residues. In contrast, recombinant human SAP (rhSAP) produced in CHO cells contains only α2,3-linked sialic acid residues. In in vitro cell-based bioassays, α2,3-linked sialic acid SAP proteins demonstrate consistently higher activity than wild-type SAP (i.e., α2,6-linked sialic acid) isolated from human serum. The variant SAP proteins of the disclosure would be more effective as therapeutic agents due to their increased biological potency. For example, more potent SAP variants may require lower dosing and/or less frequent dosing relative to wild-type SAP isolated from human serum. The disclosure provides both variant human SAP proteins and methods for making the same. In particular, the present disclosure includes methods and compositions for in vitro and in vivo addition, deletion, or modification of sugar residues to produce a human SAP protein having a desired glycosylation pattern.

### Variant SAP Proteins

In part, the disclosure provides variant Serum Amyloid P (SAP) polypeptides for use in treatment of myeloproliferative disorders. In particular, SAP variants of the disclosure include glycosylated human SAP proteins that comprise one or more N-linked or O-linked oligosaccharide chains each independently having one, two, three, four, or five branches terminating with an α2,3-linked sialic acid moiety. In some embodiments, all the sialylated branches of the N-linked or O-linked oligosaccharide chains terminate in α2,3-linked moieties. In some embodiments, 15%, 20%, 25%, 30%, 35%, 40%, 45%, 50%, 55%, 60%, 65% 75%, 80%, 85%, or even at least 95% of the sialylated branches of the N-linked or O-linked oligosaccharide chains terminate in α2,3-linked moieties. Other SAP variants of the disclosure include glycosylated human SAP proteins that contain an N-linked or O-linked oligosaccharide chains having at least 20%, 250%, 30%, 35%, 40%, 45%, 50%, 55%, 60%, 65% 75%, 80%, 85%, or even at least 95% fewer α2,6-linked sialic acid moieties than a wild-type SAP protein derived from human serum. In some embodiments, the N-linked or O-linked oligosaccharide chains are substantially free of α2,6-linked sialic acid moieties. Glycovariant SAP proteins of the disclosure may comprise an N-linked oligosaccharide or O-linked chain having one or more branches (e.g., having a bi-antennary, tri-antennary, tetra-antennary, penta-antennary, etc. structure). In certain embodiments, SAP proteins of the disclosure comprise an N-linked or O-linked oligosaccharide chain wherein one, two, three, four, or five branches of the oligosaccharide chain are substantially free of galactose and N-acetylglucosamine. Certain SAP proteins of the disclosure have N-linked or O-linked oligosaccharide chains that are substantially free of galactose and N-acetylglucosamine. In some embodiments, SAP proteins of the disclosure comprise an N-linked or O-linked oligosaccharide chain wherein one, two, three, four, or five branches of the oligosaccharide chain contain one or more mannose residues. In certain embodiments, the SAP protein of the disclosure comprises an N-linked oligosaccharide having a pentasaccharide core of Man[(α1,6-)-(Man(α1,3)]-Man(β1,4)-GlcNAc(β1,4)-GlcNAc(β1,N)-Asn. This pentasaccharide core also may comprise one or more fucose or xylose residues. In certain embodiments, SAP proteins of the disclosure comprise an N-linked oligosaccharide chain wherein one, two, three, four, or five branches of the oligosaccharide chain have the structure NeuNAc2α3Galβ4GlcNAcβ2Manα6. SAP proteins of the disclosure also may comprise an N-linked oligosaccharide chain wherein all branches have the structure NeuNAc2α3Galβ4GlcNAcβ2Manα6.

Variant SAP proteins of the disclosure may comprise one or more “modified” sugar residues. Modified sugars are substituted at any position that allows for the attachment of the modifying moiety or group, yet which still allows the sugar to function as a substrate for the enzyme used to couple the modified sugar to the peptide. A modifying group can be attached to a sugar moiety by enzymatic means, chemical means or a combination thereof, thereby producing a modified sugar, e.g., modified galactose, fucose, or sialic acid. Modifying groups suitable for use in the present disclosure as well as methods for conjugating these modifying groups to sugar residues are described in the following section.

In some embodiments, the SAP proteins of the disclosure may comprise amino acid sequences at least 60%, at least 70%, at least 80%, at least 85%, at least 90%, at least 95%, at least 96%, at least 97%, at least 98%, at least 99%, or 100% identical to the amino acid sequence of SEQ ID NO: 1, as determined using the FASTDB computer program based on the algorithm of Brutlag et al. (Comp. App. Biosci., 6:237-245 (1990)). In a specific embodiment, parameters employed to calculate percent identity and similarity of an amino acid alignment comprise: Matrix=PAM 150, k-tuple=2, Mismatch Penalty=1. Joining Penalty=20, Randomization Group Length=0, Cutoff Score=1, Gap Penalty=5 and Gap Size Penalty=0.05.

Polypeptides sharing at least 95% identity with SEQ ID NO: 1 may include polypeptides having conservative substitutions in these areas of divergence. The term “SAP protein” encompasses functional fragments and fusion proteins comprising any of the preceding. Generally, an SAP protein will be soluble in aqueous solutions at biologically relevant temperatures, pH levels and osmolarity. The SAP protomers that non-covalently associate together to form a pentameric SAP complex may have identical amino acid sequences and/or post-translational modifications or, alternatively, individual SAP protomers within a single complex may have different sequences and/or modifications. The term SAP protein includes polypeptides comprising any naturally occurring SAP protein as well as any variant thereof (including mutants, fragments, and fusions). An SAP protein of the disclosure may be a recombinant polypeptide. In preferred embodiments, the SAP protein of the disclosure is a human SAP protein.

In some embodiments, pharmaceutical compositions are provided comprising a variant SAP protein of the disclosure, or a functional fragment thereof. In some aspects, the amino acid sequence of an SAP variant may differ from SEQ ID NO: 1 by one or more conservative or non-conservative substitutions. In other aspects, the amino acid sequence of an SAP variant may differ from SEQ ID NO: 1 by one or more conservative substitutions. As used herein, “conservative substitutions” are residues that are physically or functionally similar to the corresponding reference residues, i.e., a conservative substitution and its reference residue have similar size, shape, electrical charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure 5:345-352 (1978 & Supp.). Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. Additional guidance concerning which amino acid changes are likely to be phenotypically silent can be found in Bowie et al., Science 247:1306-1310 (1990).

Variant SAP proteins and fragments thereof that retain biological function are useful in the pharmaceutical compositions and methods described herein. In some embodiments, a variant SAP protein or fragment thereof binds to one or more Fcγ receptors. In some embodiments, the Fcγ receptor is FcγRI, FcγRIIA, and/or FcγRIIIB. In some embodiments, a variant SAP protein or fragment thereof inhibits one or more of fibrocyte, fibrocyte precursor, myofibroblast precursor, and/or hematopoetic monocyte precursor differentiation. In some embodiments, a variant SAP protein or fragment thereof inhibits the differentiation of monocytes into fibrocytes. Measuring the expression of Macrophage Derived Chemokine (MDC) is an effective method of determining fibrocyte differentiation. SAP variants may be generated by modifying the structure of an SAP protein for such purposes as enhancing therapeutic efficacy or stability (e.g., ex vivo shelf life and resistance to proteolytic degradation in vivo).

In certain aspects, the variant SAP proteins of the disclosure may further comprise post-translational modifications in addition to any that are naturally present in the SAP protein. Such modifications include, but are not limited to, acetylation, carboxylation, glycosylation (e.g., O-linked oligosaccharides, N-linked oligosaccharides, etc.), phosphorylation, lipidation, and acylation. As a result, the modified SAP protein may contain non-amino acid elements, such as polyethylene glycols, lipids, poly- or mono-saccharides, and phosphates.

Methods of producing variant hSAP proteins with altered N-glycosylation are described in U.S. patent application Ser. No. 12/794,132, which is hereby incorporated by reference. In some embodiments, one or more protomers of variant SAP proteins comprise an amino acid at position 32 of SEQ ID NO: 1 that is not asparagine, resulting in altered glycosylation patterns. In some embodiments, one or more of the SAP promoters are substantially free of N-linked or O-linked glycans.

In certain aspects, one or more modifications to the SAP protein described herein may enhance the stability of the SAP protein. For example, such modifications may enhance the in vivo half-life of the SAP protein or reduce proteolytic degradation of the SAP protein.

In certain aspects, variant SAP proteins of the disclosure include fusion proteins having at least a portion of the human SAP protein and one or more fusion domains or heterologous portions. Well known examples of such fusion domains include, but are not limited to, polyhistidine, Glu-Glu, glutathione S transferase (GST), thioredoxin, protein A, protein G, and immunoglobulin heavy chain constant region (Fe), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel-, or cobalt-conjugated resins are used. As another example, a fusion domain may be selected so as to facilitate detection of the SAP proteins. Examples of such detection domains include the various fluorescent protein (e.g., GFP) as well as “epitope tags,” which are usually short peptide sequences for which a specific antibody is available. Well known epitope tags for which specific monoclonal antibodies are readily available include FLAG, influenza virus hemagglutinin (HA) and c-myc tags. In some cases, the fusion domains have a protease cleavage site that allows the relevant protease to partially digest the fusion proteins and thereby liberate the recombinant protein therefrom. The liberated proteins can then be isolated from the fusion domain by subsequent chromatographic separation. In some cases, the SAP protein may be fused to a heterologous domain that stabilizes the SAP protein in vivo. By “stabilizing” is meant anything that increases serum half-life, regardless of whether this is because of decreased destruction, decreased clearance by the liver and/or kidney, or other pharmacokinetic effect. Fusions with the Fc portion of an immunoglobulin and serum albumin are known to confer increased stability.

It is understood that different elements of the fusion proteins may be arranged in any manner that is consistent with the desired functionality. For example, an SAP protein may be placed C-terminal to a heterologous domain, or, alternatively, a heterologous domain may be placed C-terminal to an SAP protein. The SAP protein and the heterologous domain need not be adjacent in a fusion protein, and additional domains or amino acid sequences (e.g., linker sequences) may be included C- or N-terminal to either domain or between the domains.

SAP proteins of the disclosure may comprise one or more “modified” sugar residues. A modifying group can be attached to a sugar moiety by enzymatic means, chemical means or a combination thereof, thereby producing a modified sugar, e.g., modified galactose, fucose, or sialic acid. When a modified sialic acid is used, either a sialyltransferase or a trans-sialidase can be used in these methods. The sugars may be substituted at any position that allows for the attachment of the modifying moiety, yet which still allows the sugar to function as a substrate for the enzyme used to couple the modified sugar to the peptide.

In general, the sugar moiety and the modifying group are linked together through the use of reactive groups, which are typically transformed by the linking process into a new organic functional group or unreactive species. The sugar reactive functional group(s) may be located at any position on the sugar moiety. Reactive groups and classes of reactions useful in practicing the present disclosure are generally those that are well known in the art of bioconjugate chemistry. Currently favored classes of reactions available with reactive sugar moieties are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, Smith and March. Advanced Organic Chemistry, 5th Ed., John Wiley & Sons, New York, 2001; Hermanson, Bioconjugate Techniques, Academic Press, San Diego, 1996; and Feeney et al., Modification of Proteins; Advances in Chemistry Series, Vol. 198. American Chemical Society, Washington, D.C., 1982.

Useful reactive functional groups pendent from a sugar nucleus or modifying group include, but are not limited to: (a) carboxyl groups and various derivatives thereof (e.g., N-hydroxysuccinimide esters, N-hydroxybenzotriazole esters, acid halides, acyl imidazoles, thioesters, p-nitrophenyl esters, alkyl, alkenyl, alkynyl and aromatic esters); (b) hydroxyl groups, which can be converted to, e.g., esters, ethers, aldehydes, etc.; (c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the functional group of the halogen atom; (d) dienophile groups, which are capable of participating in Diels-Alder reactions such as, for example, maleimido groups (e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (e) thiol groups, which can be, for example, converted to disulfides or reacted with alkyl and acyl halides; (h) amine or sulfhydryl groups, which can be, for example, acylated, alkylated or oxidized; (i) alkenes, which can undergo, for example, cycloadditions, acylation, Michael addition, metathesis, Heck reaction, etc.; (j) epoxides, which can react with, for example, amines and hydroxyl compounds.

The reactive functional groups can be chosen such that they do not participate in, or interfere with, the reactions necessary to assemble the reactive sugar nucleus or modifying group. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., Protective Groups in Organic Synthesis, John Wiley & Sons, New York, 1991.

In some embodiments, the modified sugar is an activated sugar. Activated modified sugars useful in the present disclosure are typically glycosides which have been synthetically altered to include an activated leaving group. As used herein, the term “activated leaving group” refers to those moieties which are easily displaced in enzyme-regulated nucleophilic substitution reactions. Many activated sugars are known in the art. See, for example. Vocadlo et al., In Carbohydrate Chemistry and Biology, Vol. 2, Ernst et al. Ed., Wiley-VCH Verlag: Weinheim, Germany, 2000; Kodama et al., Tetrahedron Lett. 34: 6419 (1993); Lougheed, et al., J. Biol. Chem. 274: 37717 (1999)). Examples of such leaving groups include fluoro, chloro, bromo, tosylate, mesylate, triflate and the like. Preferred activated leaving groups for use in the present disclosure are those that do not significantly sterically encumber the enzymatic transfer of the glycoside to the acceptor. Accordingly, preferred embodiments of activated glycoside derivatives include glycosyl fluorides and glycosyl mesylates, with glycosyl fluorides being particularly preferred. Among the glycosyl fluorides, α-galactosyl fluoride, α-mannosyl fluoride, α-glucosyl fluoride, α-fucosyl fluoride, α-xylosyl fluoride, α-sialyl fluoride, α-N-acetylglucosaminyl fluoride, α-N-acetylgalactosaminyl fluoride, α-galactosyl fluoride, (β-mannosyl fluoride, β.-glucosyl fluoride, β-fucosyl fluoride, β-xylosyl fluoride. β-sialyl fluoride, β-N-acetylglucosaminyl fluoride and β-N-acetylgalactosaminyl fluoride are most preferred.

In certain aspects, a modified sugar residue is conjugated to one or more water-soluble polymers. Many water-soluble polymers are known to those of skill in the art and are useful in practicing the present disclosure. The term water-soluble polymer encompasses species such as saccharides (e.g., dextran, amylose, hyaluronic acid, poly(sialic acid), heparans, heparins, etc.); poly(amino acids); nucleic acids; synthetic polymers (e.g., poly(acrylic acid), poly(ethers), e.g., poly(ethylene glycol)); peptides, proteins, and the like. The present disclosure may be practiced with any water-soluble polymer with the sole limitation that the polymer must include a point at which the remainder of the conjugate can be attached.

Methods and chemistry for activation of water-soluble polymers and saccharides as well as methods for conjugating saccharides and polymers to various species are described in the literature. Commonly used methods for activation of polymers include activation of functional groups with cyanogen bromide, periodate, glutaraldehyde, biepoxides, epichlorohydrin, divinylsulfone, carbodiimide, sulfonyl halides, trichlorotriazine, etc. (see, R. F. Taylor, (1991), Protein Immobilisation, Fundamentals and Applications, Marcel Dekker. N.Y.; S. S. Wong, (1992), Chemistry of Protein Conjugation and Crosslinking, CRC Press, Boca Raton; G. T. Hermanson et al., (1993), Immobilized Affinity Ligand Techniques, Academic Press, N.Y.; Dunn, R. L., et al., Eds. Polymeric Drugs and Drug Delivery Systems, ACS Symposium Series Vol. 469, American Chemical Society, Washington, D.C. 1991).

In certain aspects, a modified sugar residue is conjugated to one or more water-insoluble polymers. In some embodiments, conjugation to a water-insoluble polymer can be used to deliver a therapeutic peptide in a controlled manner. Polymeric drug delivery systems are known in the art. See, for example, Dunn et al., Eds. Polymeric drugs and Drug Delivery Systems, ACS Symposium Series Vol. 469, American Chemical Society, Washington, D.C. 1991. Those of skill in the art will appreciate that substantially any known drug delivery system is applicable to the conjugates of the present disclosure.

Representative water-insoluble polymers include, but are not limited to, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, pluronics, and polyvinylphenol, and copolymers thereof.

These and the other polymers discussed herein can be readily obtained from commercial sources such as Sigma Chemical Co. (St. Louis, Mo.), Polysciences (Warrenton. Pa.), Aldrich (Milwaukee, Wis.), Fluka (Ronkonkoma, N.Y.), and BioRad (Richmond, Calif.), or else synthesized from monomers obtained from these suppliers using standard techniques. Representative biodegradable polymers useful in the conjugates of the disclosure include, but are not limited to, polylactides, polyglycolides and copolymers thereof poly(ethylene terephthalate), poly(butyric acid), poly(valeric acid), poly(lactide-co-caprolactone), poly(lactide-co-glycolide), polyanhydrides, polyorthoesters, blends and copolymers thereof. Of particular use are compositions that form gels, such as those including collagen, and pluronics.

In a preferred embodiment, one or more modified sugar residues are conjugated to one or more PEG molecules.

In certain aspects, the modified sugar is conjugated to a biomolecule. Biomolecule of the disclosure may include, but are not limited to, functional proteins, enzymes, antigens, antibodies, peptides, nucleic acids (e.g., single nucleotides or nucleosides, oligonucleotides, polynucleotides and single- and higher-stranded nucleic acids), lectins, receptors or a combination thereof.

Some preferred biomolecules are essentially non-fluorescent, or emit such a minimal amount of fluorescence that they are inappropriate for use as a fluorescent marker in an assay. Other biomolecules may be fluorescent.

In some embodiments, the biomolecule is a targeting moiety. A “targeting moiety” and “targeting agent”, as used herein, refer to species that will selectively localize in a particular tissue or region of the body. In some embodiments, a biomolecule is selected to direct the SAP protein of the disclosure to a specific intracellular compartment, thereby enhancing the delivery of the peptide to that intracellular compartment relative to the amount of underivatized peptide that is delivered to the tissue. The localization is mediated by specific recognition of molecular determinants, molecular size of the targeting agent or conjugate, ionic interactions, hydrophobic interactions and the like. Other mechanisms of targeting an agent to a particular tissue or region are known to those of still in the art.

In some embodiments, the modified sugar includes a therapeutic moiety. Those of skill in the art will appreciate that there is overlap between the category of therapeutic moieties and biomolecules, i.e., many biomolecules have therapeutic properties or potential.

Classes of useful therapeutic moieties include, for example, non-steroidal anti-inflammatory drugs; steroidal anti-inflammatory drugs; adjuvants; antihistaminic drugs; antitussive drugs; antipruritic drugs; anticholinergic drugs; anti-emetic and antinauseant drugs; anorexic drugs; central stimulant drugs; antiarrhythmic drugs; β-adrenergic blocker drugs; cardiotonic drugs; antihypertensive drugs; diuretic drugs; vasodilator drugs; vasoconstrictor drugs; antiulcer drugs; anesthetic drugs; antidepressant drugs; tranquilizer and sedative drugs; antipsychotic drugs; and antimicrobial drugs.

Other drug moieties useful in practicing the present disclosure include antineoplastic drugs, cytocidal agents, anti-estrogens, and antimetabolites. Also included within this class are radioisotope-based agents for both diagnosis (e.g., imaging) and therapy, and conjugated toxins.

The therapeutic moiety can also be a hormone, a muscle relaxant, an antispasmodic, bone activating agent, endocrine modulating agent, modulator of diabetes, androgen, antidiuretics, or calxitonin drug.

Other useful modifying groups include immunomodulating drugs, immunosuppressants, etc. Groups with anti-inflammatory activity, such as sulindac, etodolac, ketoprofen and ketorolac, are also of use. Other drugs of use in conjunction with the present disclosure will be apparent to those of skill in the art.

The altered N-glycosylation SAP proteins produced by the methods of the disclosure can be homogeneous (i.e., the sample of SAP protein is uniform in specific N-glycan structure) or substantially homogeneous. By “substantially homogeneous” is meant that at least about 25% (e.g., at least about 27%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, or at least about 99%) of the SAP proteins contain the same specific N-glycan structure.

In some embodiments, variant SAP proteins of the disclosure have an IC₅₀ for inhibiting the differentiation of monocytes into fibrocytes in vitro that is less than ½, less than ⅓, less than ¼, less than 1/10, or less than 1/100 that of a corresponding sample of wild-type SAP isolated from human serum. In some embodiments, variant SAP proteins of the disclosure have an IC₅₀ for inhibiting the differentiation of monocytes into fibrocytes in vitro that is less than one-half that of a corresponding sample of wild-type SAP isolated from human serum. There are many well characterized methods for determining the responsiveness of Peripheral Blood Mononuclear Cells (PBMCs) or monocyte cells to SAP for fibrocyte differentiation. In some embodiments, the SAP protein of the disclosure inhibits production of IL-8. In some embodiments, the inhibitory effect of an SAP protein of the disclosure to block phorbol myristate acetate (PMA)-induced production of IL-8 is measured. These methods may be used to determine the relative potency of any of the SAP variant polypeptides of the disclosure in comparison to a sample of human serum-derived SAP, any other SAP variant polypeptide, or other fibrocyte suppressant or activating agent. PBMCs or monocytes suitable for use in these methods may be obtained from various tissue culture lines. Alternatively, suitable cells for fibrocyte differentiation assays may be obtained from any biological sample that contains PBMC or monocyte cells. The biological sample may be obtained from serum, plasma, healthy tissue, or fibrotic tissue. In general, fibrocyte differentiation assays are conducted by incubating PBMC or monocyte cells in media with various concentrations of an SAP protein to determine the degree of fibrocyte differentiation. The concentration of SAP can range from 0.0001 μg/mL to 1 mg/ml, and in some embodiments is 0.001 μg/mL, 1.0 μg/mL, 5 μg/mL, 10 μg/mL, 15 μg/mL, 20 μg/mL, 25 μg/mL, 30 μg/mL, 35 μg/mL, 40 μg/mL, 45 μg/mL, 50 μg/mL, 100 μg/mL, 200 μg/mL, 300 μg/mL, or 500 μg/mL. In some assays, the media can be supplemented with between 1-100 ng/ml hMCSF; the preferred concentration of hMCSF being 25 ng/mL. The indication that PBMC and monocytes have differentiated into fibrocytes can be determined by one skilled in the art. In general, fibrocytes are morphologically defined as adherent cells with an elongated spindle-shape and the presence of an oval nucleus. In some assays, cells are fixed and stained with Hema 3 before enumerating fibrocytes by direct counting, e.g., using an inverted microscope. The amount of fibrocyte differentiation is interpreted by one skilled in the art as an indication of a cell's responsiveness to SAP. A greater suppression of fibrocyte differentiation indicates a greater degree of SAP responsiveness. An alternative method of measuring fibrocyte differentiation involves determining the expression of fibrocyte-specific cell surface markers or secreted factors, e.g., cytokines (such as IL-1ra, ENA-78/CXCL-5, PAI-1), fibronectin, collagen-1). Methods of detecting and/or quantifying cell surface markers or secreted factors are well known in the art, including but not limited to various ELISA- and FACS-based techniques using immunoreactive antibodies against one or more fibrocyte-specific markers. Measuring the expression of Macrophage Derived Chemokine (MDC) is an effective method of determining fibrocyte differentiation.

Methods for detecting and/or characterizing N-glycosylation (e.g., altered N-glycosylation) of an SAP protein include DNA sequencer-assisted (DSA), fluorophore-assisted carbohydrate electrophoresis (FACE) or surface-enhanced laser desorption/ionization time-of-flight mass spectrometry (SELDI-TOF MS). For example, an analysis can utilize DSA-FACE in which, for example, glycoproteins are denatured followed by immobilization on, e.g., a membrane. The glycoproteins can then be reduced with a suitable reducing agent such as dithiothreitol (DTT) or β-mercaptoethanol. The sulfhydryl groups of the proteins can be carboxylated using an acid such as iodoacetic acid. Next, the N-glycans can be released from the protein using an enzyme such as N-glycosidase F. N-glycans, optionally, can be reconstituted and derivatized by reductive amination. The derivatized N-glycans can then be concentrated. Instrumentation suitable for N-glycan analysis includes, for example, the ABI PRISM® 377 DNA sequencer (Applied Biosystems). Data analysis can be performed using, for example, GENESCAN® 3.1 software (Applied Biosystems). Optionally, isolated mannoproteins can be further treated with one or more enzymes to confirm their N-glycan status. Exemplary enzymes include, for example, α-mannosidase or α-1,2 mannosidase. Additional methods of N-glycan analysis include, for example, mass spectrometry (e.g., MALDI-TOF-MS), high-pressure liquid chromatography (HPLC) on normal phase, reversed phase and ion exchange chromatography (e.g., with pulsed amperometric detection when glycans are not labeled and with UV absorbance or fluorescence if glycans are appropriately labeled). See also Callewaert et al. (2001) Glycobiology 11(4):275-281 and Freire et al. (2006) Bioconjug. Chem. 17(2):559-564, the disclosures of each of which are incorporated herein by reference in their entirety.

### Anti-FcγR Antibodies as SAP Agonists

In one aspect of the disclosure, one or more compounds are provided that mimic SAP signaling. In some embodiments, the SAP signaling agonists are anti-FcγR antibodies, wherein the antibodies are selected from a class of anti-FcγRI, anti-FcγRIIA, and anti-FcγRIII antibodies that are able to bind to either FcγRI, FcγRIIA, or FcγRIII, respectively. Anti-FcγR antibodies are antibodies that bind to receptors for the Fc portion of IgG antibodies (FcγR). The anti-FcγR antibodies bind through their variable region, and not through their constant (Fc) region. Anti-FcγR antibodies may include any isotype of antibody. The anti-FcγR antibodies may be further cross-linked or aggregated with or without additional antibodies or other means. This process initiates intracellular signaling events consistent with FcγR activation. In some embodiments, the SAP signaling agonist may be a cross-linked FcγR.

### Aggregated Fc Domains and Fc-Containing Antibodies

In some embodiments, the SAP signaling agonists are cross-linked or aggregated IgG. Cross-linked or aggregated IgG may include any IgG able to bind the target FcγR through its Fc region, provided that at least two such IgG antibodies are physically connected to one another.

Cross-linked or aggregated IgG may include whole antibodies or a portion thereof, preferably the portion functional in suppression of fibrotic disorders. For example, they may include any antibody portion able to cross-link FcγR. This may include aggregated or cross-linked antibodies or fragments thereof, such as aggregated or cross-linked whole antibodies, Fab fragments, F(ab′)₂ fragments, Fab′ fragments, and possibly even Fc fragments.

Aggregation or cross-linking of antibodies may be accomplished by any known method, such as heat or chemical aggregation. Any level of aggregation or cross-linking may be sufficient, although increased aggregation may result in increased fibrotic disorder suppression. Antibodies may be polyclonal or monoclonal, such as antibodies produced from hybridoma cells. Compositions and methods may employ mixtures of antibodies, such as mixtures of multiple monoclonal antibodies, which may be cross-linked or aggregated to like or different antibodies.

### SAP Peptidomimetic

In certain embodiments, the SAP agonists include peptidomimetics. As used herein, the term “peptidomimetic” includes chemically modified peptides and peptide-like molecules that contain non-naturally occurring amino acids, peptoids, and the like. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems. San Leandro Calif.), contains about 100,000 compounds that are commercially available and also can be searched to identify potential peptidomimetics of SAP proteins.

### Increase SAP Activity

In some embodiments, an SAP agonist increases SAP activity. SAP activity can be increased by increasing the concentration of SAP by, for example, increasing SAP transcription, increasing translation, increasing SAP secretion, increasing SAP RNA stability, increasing SAP protein stability, or decreasing SAP protein degradation. SAP activity can also be increased by increasing specifically the “free concentration” of SAP, or rather the unbound form by, for example, decreasing SAP endogenous binding partners.

### FcγR Crosslinkers

In some embodiments, fibronectin-based scaffold domain proteins may be used as SAP agonists to crosslink FcγRs. Fibronectin-based scaffold domain proteins may comprise a fibronectin type III domain (Fn3), in particular a fibronectin type III tenth domain (¹⁰Fn3).

In order to crosslink FcγRs, multimers of FcγR binding Fn3 domains may be generated as described in U.S. Pat. No. 7,115,396.

Fibronectin type III (Fn3) domains comprise, in order from N-terminus to C-terminus, a beta or beta-like strand, A; a loop. AB; a beta or beta-like strand, B; a loop, BC; a beta or beta-like strand C; a loop CD; a beta or beta-like strand D; a loop DE; a beta or beta-like strand. E; a loop, EF; a beta or beta-like strand F; a loop FG; and a beta or beta-like strand G. The BC, DE, and FG loops are both structurally and functionally analogous to the complementarity-determining regions (CDRs) from immunoglobulins Fn3 domains can be designed to bind almost any compound by altering the sequence of one or more of the BC, DE, and FG loops. Methods for generating specific binders have been described in U.S. Pat. No. 7,115,396, disclosing high affinity TNFα binders, and U.S. Publication No. 2007/0148126, disclosing high affinity VEGFR2 binders. An example of fibronectin-based scaffold proteins are Adnectins™ (Adnexus, a Bristol-Myers Squibb R&D Company).

In some embodiments, the SAP agonist is an aptamer. In order to crosslink FcγRs, multimers of FcγR binding aptamers may be generated.

Aptamers are oligonucleotides, which can be synthetic or natural, that bind to a particular target molecule, such as a protein or metabolite. Typically, the binding is through interactions other than classic Watson-Crick base pairing. Aptamers represent a promising class of therapeutic agents currently in pre-clinical and clinical development. Like biologics, e.g., peptides or monoclonal antibodies, aptamers are capable of binding specifically to molecular targets and, through binding, inhibiting target function. A typical aptamer is 10-15 kDa in size (i.e., 30-45 nucleotides), binds its target with sub-nanomolar affinity, and discriminates among closely related targets (e.g., will typically not bind other proteins from the same gene family) (Griffin, et al. (1993), Gene 137(1): 25-31; Jenison, et al. (1998), Antisense Nucleic Acid Drug Dev. 8(4): 265-79; Bell, et al. (1999), In vitro Cell. Dev. Biol. Anim 35(9): 533-42; Watson, et al. (2000). Antisense Nucleic Acid Drug Dev. 10(2): 63-75; Daniels, et al. (2002), Anal. Biochem. 305(2): 214-26; Chen, et al. (2003), Proc. Natl. Acad. Sci. U.S.A. 100(16): 9226-31; Khati, et al. (2003), J. Virol. 77(23): 12692-8; Vaish, et al. (2003). Biochemistry 42(29): 8842-51).

Aptamers have a number of attractive characteristics for use as therapeutics. In addition to high target affinity and specificity, aptamers have shown little or no toxicity or immunogenicity in standard assays (Wlotzka. et al. (2002), Proc. Natl. Acad. Sci. U.S.A. 99(13): 8898-902). Indeed, several therapeutic aptamers have been optimized and advanced through varying stages of pre-clinical development, including pharmacokinetic analysis, characterization of biological efficacy in cellular and animal disease models, and preliminary safety pharmacology assessment (Reyderman and Stavchansky (1998), Pharmaceutical Research 15(6): 904-10; Tucker et al., (1999), J. Chromatography B. 732: 203-212; Watson, et al. (2000), Antisense Nucleic Acid Drug Dev. 10(2): 63-75).

A suitable method for generating an aptamer to a target of interest is with the process entitled “Systematic Evolution of Ligands by EXponential Enrichment” (“SELEX™”). The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX™-identified nucleic acid ligand is a specific ligand of a given target compound or molecule. The SELEX™ process is based on the insight that nucleic acids can form a variety of two- and three-dimensional structures and have sufficient chemical versatility available within their monomers to act as ligands (form specific binding pairs) with virtually any chemical compound, whether monomeric or polymeric. Molecules of any size or composition can serve as targets. The SELEX™ method applied to the application of high affinity binding involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX™ method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific high affinity nucleic acid ligands to the target molecule. SELEX™ is a method for making a nucleic acid ligand for any desired target, as described, e.g., in U.S. Pat. Nos. 5,475,096 and 5,270,163, and PCT/US91/04078, each of which is specifically incorporated herein by reference.

In some embodiments, SAP agonists are Nanobodies®. Nanobodies® are antibody-derived therapeutic proteins that contain the unique structural and functional properties of naturally-occurring heavy-chain antibodies. The Nanobody® technology was originally developed following the discovery that camelidae (camels and llamas) possess fully functional antibodies that lack light chains. These heavy-chain antibodies contain a single variable domain (VHH) and two constant domains (CH2 and CH3). Importantly, the cloned and isolated VHH domain is a stable polypeptide harboring the full antigen-binding capacity of the original heavy-chain antibody. These VHH domains with their unique structural and functional properties form the basis of a new generation of therapeutic antibodies.

### Pharmaceutical Preparations and Formulations

In certain embodiments, the methods described herein involve administration of at least one SAP protein of the disclosure to a subject as a therapeutic agent. The therapeutic agents of the disclosure may be formulated in a conventional manner using one or more physiologically acceptable carriers or excipients. For example, therapeutic agents and their physiologically acceptable salts and solvates may be formulated for administration by, for example, intravenous infusion (IV), injection (e.g. SubQ, IM, IP), inhalation or insufflation (either through the mouth or the nose) or oral, buccal, sublingual, transdermal, nasal, parenteral or rectal administration. In certain embodiments, therapeutic agents may be administered locally, at the site where the target cells are present, i.e., in a specific tissue, organ, or fluid (e.g., blood, cerebrospinal fluid, tumor mass, etc.). In other words, the disclosure contemplates that for any of the methods described herein, the method comprises administration of SAP or a composition comprising SAP (e.g., a pharmaceutical composition). In certain preferred embodiments, the composition is a composition comprising SAP, wherein the activity and/or SAP sialyation across the composition differs from that in human serum.

The present disclosure further provides use of any SAP protein of the disclosure in the manufacture of a medicament for the treatment or prevention of a disorder or a condition, as described herein, in a patient, for example, the use of an SAP protein in the manufacture of medicament for the treatment of a disorder or condition described herein. In some aspects, an SAP protein of the disclosure may be used to make a pharmaceutical preparation for the use in treating or preventing a disease or condition described herein.

Therapeutic agents can be formulated for a variety of modes of administration, including systemic and topical or localized administration. Techniques and formulations generally may be found in Remington's Pharmaceutical Sciences, Meade Publishing Co., Easton, Pa. For parenteral administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included. In some embodiments, the therapeutic agents can be administered to cells by a variety of methods known to those familiar in the art, including, but not restricted to, encapsulation in liposomes, by iontophoresis, or by incorporation into other vehicles, such as hydrogels, cyclodextrins, biodegradable nanocapsules, and bioadhesive microspheres.

For oral administration, the pharmaceutical compositions may take the form of, for example, tablets, lozenges, or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation (e.g., pulmonary delivery), therapeutic agents may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

In the methods of the disclosure, the pharmaceutical compounds can also be administered by intranasal or intrabronchial routes including insufflation, powders, and aerosol formulations (for examples of steroid inhalants, see Rohatagi (1995) J. Clin. Pharmacol. 35:1187-1193; Tjwa (1995) Ann. Allergy Asthma Immunol. 75:107-111). For example, aerosol formulations can be placed into pressurized acceptable propellants, such as dichlorodifluoromethane, propane, nitrogen, and the like. They also may be formulated as pharmaceuticals for non-pressured preparations such as in a nebulizer or an atomizer. Typically, such administration is in an aqueous pharmacologically acceptable buffer.

Pharmaceutical compositions suitable for respiratory delivery (e.g., intranasal, inhalation, etc.) of SAP proteins may be prepared in either solid or liquid form.

SAP proteins of the disclosure may be formulated for parenteral administration by injection, e.g., by bolus injection or continuous infusion. In certain embodiments, the SAP proteins are formulated for intravenous delivery. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

SAP proteins of the disclosure may be formulated for subcutaneous delivery, e.g., by injection. Formulations for injection may be presented in unit dosage form, e.g., in ampoules or in multi-dose containers, with or without an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In addition, SAP proteins of the disclosure may also be formulated as a depot preparation. Such long-acting formulations may be administered by implantation (for example subcutaneously or intramuscularly) or by intramuscular injection. Thus, for example, therapeutic agents may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches.

In certain embodiments, SAP proteins of the disclosure are incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Pharmaceutical compositions (including cosmetic preparations) may comprise from about 0.00001 to 100% such as from 0.001 to 10% or from 0.1% to 5% by weight of one or more of the SAP proteins described herein. In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Therapeutic agents described herein may be stored in oxygen-free environment according to methods in the art.

Exemplary compositions comprise an SAP protein with one or more pharmaceutically acceptable carriers and, optionally, other therapeutic ingredients. The carrier(s) must be “pharmaceutically acceptable” in the sense of being compatible with the other ingredients of the composition and not eliciting an unacceptable deleterious effect in the subject. Such carriers are described herein or are otherwise well known to those skilled in the art of pharmacology. In some embodiments, the pharmaceutical compositions are pyrogen-free and are suitable for administration to a human patient. In some embodiments, the pharmaceutical compositions are irritant-free and are suitable for administration to a human patient. In some embodiments, the pharmaceutical compositions are allergen-free and are suitable for administration to a human patient. The compositions may be prepared by any of the methods well known in the art of pharmacy.

In some embodiments, an SAP protein is administered in a time release formulation, for example in a composition which includes a slow release polymer. An SAP protein can be prepared with carriers that will protect against rapid release. Examples include a controlled release vehicle, such as a polymer, microencapsulated delivery system, or bioadhesive gel. Alternatively, prolonged delivery of an SAP protein may be achieved by including in the composition agents that delay absorption, for example, aluminum monostearate hydrogels and gelatin.

In certain embodiments, the methods of the disclosure comprise administration via any of the foregoing routes of administration, such as intravenous or subcutaneous. In certain embodiments, administration is subcutaneous, particularly when each dose being administered is in a small volume. In certain embodiments, administration is intravenous.

The following examples serve to more fully describe the manner of using the above-described disclosure, as well as to set forth the best modes contemplated for carrying out various aspects of the disclosure. It is understood that these examples in no way serve to limit the true scope of this disclosure, but rather are presented for illustrative purposes.

### EXEMPLIFICATION

### Example 1. Treatment of Myelofibrosis with Recombinant Human SAP (rhSAP)

Patients diagnosed as having myelofibrosis, including PMF, post-PV MF, or post ET-MF are tested for their baseline mutational status in one or more genes such as JAK2, MPL, CALR, ASXL1, EZH2, SRSF2, IDH1, and IDH2. To measure the baseline mutational status, peripheral blood samples are collected from the patients and DNA is extracted from the samples and analyzed by real-time quantitative allele-specific PCR to measure the mutational status (i.e., identify the presence or absence of one or more mutations) of one or more genes such as JAK2, MPL, CALR, ASXL1, EZH2, SRSF2, IDH1, or IDH2. Patients receive human, α2,3-sialic acid-containing SAP recombinantly expressed in CHO cells (rhSAP expressed in CHO cells; SAP comprising at least one α2,3 linkage and differing in glycosylation from SAP derived from human serum; an exemplary SAP protein of the disclosure). Efficacy is assessed by evaluation of the bone marrow response rate, defined as a reduction of one grade in the WHO myelofibrosis grade as described in the EU Consensus Criteria. Response rate to SAP treatment will be correlated to the mutational status of the patient. This method can be used to identify subpopulations of myelofibrosis patients (e.g., patients with one or more specific combinations of mutations in genes such as JAK2, MPL, CALR, ASXL1, EZH2, SRSF2, IDH1, and IDH2) who are particularly appropriate for treatment with rhSAP. Further, change in mutational status of one or more genes such as JAK2, MPL, CALR, ASXL1, EZH2, SRSF2, IDH1, and IDH2 is measured at regular intervals to evaluate any difference in responsiveness or change in allele burden. The dosage regimen is maintained if a reduction in allele burden in one or more genes is observed. Subjects responding to therapy will continue receiving it as long as there is a benefit.

### Example 2. Treatment of Myelofibrosis with Recombinant Human SAP (rhSAP)

Patients diagnosed as having myelofibrosis, including PMF, post-PV MF, or post ET-MF are tested for their baseline mutational status in one or more genes such as JAK2, MPL, CALR, ASXL1, EZH2, SRSF2, IDH1, and IDH2. To measure the baseline mutational status, peripheral blood samples are collected from the patients and DNA is extracted from the samples and analyzed by real-time quantitative allele-specific PCR to measure the mutational status (i.e., identify the presence or absence of one or more mutations) of one or more genes such as JAK2, MPL, CALR, ASXL1, EZH2, SRSF2, IDH1, or IDH2. Patients receive human, α2,3-sialic acid-containing SAP recombinantly expressed in CHO cells (rhSAP expressed in CHO cells, SAP comprising at least one α2,3 linkage and differing in glycosylation from SAP derived from human serum). Efficacy is assessed by evaluation of the bone marrow response rate, defined as a reduction of one grade in the WHO myelofibrosis grade as described in the EU Consensus Criteria. Change in mutational status of one or more genes such as JAK2, MPL, CALR, ASXL1, EZH2, SRSF2, IDH1, and IDH2 is measured at regular intervals to evaluate any difference in responsiveness or change in allele burden. If no change in the allele burden in any of the tested genes is observed, the dosage regimen is modified to increase the dosage of rhSAP and/or increase the frequency of rhSAP administration.

### Example 3. Reduction of Mutant Allele Burden in Myelofibrosis with Recombinant Human SAP (rhSAP)

Patients diagnosed as having myelofibrosis, including PMF, post-PV MF, or post ET-MF are tested for their baseline mutational status in one or more genes such as JAK2, MPL, CALR, ASXL1, EZH2, SRSF2, IDH1, and IDH2. To measure the baseline mutational status, peripheral blood samples are collected from the patients and DNA is extracted from the samples and analyzed by real-time quantitative allele-specific PCR to measure the mutational status (i.e., identify the presence or absence of one or mutations) of one or more genes such as JAK2, MPL, CALR, ASXL1, EZH2, SRSF2, IDH1, or IDH2. Patients who have a mutation in one or more genes receive human, α2,3-sialic acid-containing SAP recombinantly expressed in CHO cells (rhSAP expressed in CHO cells; SAP comprising at least one α2,3 linkage and differing in glycosylation from SAP derived from human serum). Dosage is adjusted to be effective to reduce mutant allele burden in one or more genes. Subjects responding to therapy continue receiving it as long as there is a benefit.

### Example 4. Reduction of Mutant Allele Burden as an Indicator of Treatment Efficacy with Recombinant Human SAP (rhSAP)

Patients diagnosed as having myelofibrosis, including PMF, post-PV MF, or post ET-MF are tested for their baseline mutational status in one or more genes such as JAK2, MPL, CALR, ASXL1, EZH2, SRSF2, IDH1, and IDH2. To measure the baseline mutational status, peripheral blood samples are collected from the patients and DNA is extracted from the samples and analyzed by real-time quantitative allele-specific PCR to measure the allele burden of one or more genes such as JAK2, MPL, CALR, ASXL1, EZH2, SRSF2, IDH1, or IDH2. Patients receive human, α2,3-sialic acid-containing SAP recombinantly expressed in CHO cells (rhSAP expressed in CHO cells; SAP comprising at least one α2,3 linkage and differing in glycosylation from SAP derived from human serum). A second mutant allele burden of the same mutation is measured after administration of the SAP protein. A decrease in the second mutant allele burden relative to the first mutant allele burden indicates that the administration of the SAP protein is effective in treating the myeloproliferative disorder. Mutant allele burden may be measured at one or more time points following initiation of treatment, such as after about one month, two months, three months, four months or five months of treatment. Mutant allele levels may be subsequently monitored to evaluate durability of response.

### Example 5. Treatment of Myelofibrosis Patients with Mutations in One or More of JAK2, MPL, CALR, ASXL1, EZH2, SRSF2, IDH1, and IDH2 with Recombinant Human SAP (rhSAP)

Patients diagnosed as having myelofibrosis, including PMF, post-PV MF, or post ET-MF are tested for their mutational status in one or more genes such as JAK2, MPL, CALR, ASXL1, EZH2, SRSF2, IDH1, and IDH2. To measure the baseline mutational status, peripheral blood samples are collected from the patients and DNA is extracted from the samples and analyzed by real-time quantitative allele-specific PCR to measure the mutational status (i.e., identify the presence or absence of one or mutations) of one or more genes such as JAK2, MPL, CALR, ASXL1, EZH2, SRSF2, IDH1, or IDH2. Patients who carry a mutation in one or more of the genes receive human, α2,3-sialic acid-containing SAP recombinantly expressed in CHO cells (rhSAP expressed in CHO cells; SAP comprising at least one α2,3 linkage and differing in glycosylation from SAP derived from human serum). Efficacy is assessed by evaluation of the bone marrow response rate, defined as a reduction of one grade in the WHO myelofibrosis grade as described in the EU Consensus Criteria. Subjects responding to therapy continue receiving it as long as there is a benefit. Optionally, additional criteria are measured, such as hemoglobin, platelet count, symptoms, mutant allele status and the like. Any one or more of these criteria may be measured at one or more time points following initiation of treatment, such as after about one month, two months, three months, four months or five months of treatment. Any one or more of these criteria may be subsequently monitored to evaluate durability of response.

### Example 6. Treatment of Myelofibrosis Patients with PRM-151 Alone: Stage 2

This study evaluates an every four week dosing schedule, following a loading period. Recombinant human SAP, in this case the recombinant human SAP known as PRM-151, is administered to intermediate-2 or high risk patients with PMF, post-PV PMF, or post-ET PMF to evaluate safety and efficacy of three different doses of PRM-151 in reducing bone marrow fibrosis by ≥1 grade. Patients who are anemic or thrombocytopenic and are not receiving therapy for MF other than transfusions are eligible for this study. Patients are not candidates for ruxolitinib based on either a platelet count <50×10⁹/L or Hgb <100 g/L, have received ≥2 units PRBC in the 12 weeks prior to study entry, and be intolerant of or had inadequate response to ruxolitinib.

84 patients with intermediate-2 or high risk MF who meet the eligibility requirements are randomized to one of 3 groups receiving treatment with single agent PRM-151. Group 1: patients who have received no MF-directed drug treatment for MF in at least four weeks receive (i) an initial loading dose of PRM-151 at 0.3 mg/kg by intravenous infusion on days 1, 3, and 5 of cycle 1 (a 28-day cycle), and (ii) thereafter were administered a dose of PRM-151 at 0.3 mg/kg by intravenous infusion on day 1 of each subsequent 28-day cycle for nine cycles. Group 2: patients who have received no MF-directed drug treatment for MF in at least four weeks receive (i) an initial loading dose of PRM-151 at 3 mg/kg by intravenous infusion on days 1, 3, and 5 of cycle 1 (a 28-day cycle), and (ii) thereafter were administered a dose of PRM-151 at 3 mg/kg by intravenous infusion on day 1 of each subsequent 28-day cycle for nine cycles. Group 3: patients who have received no MF-directed drug treatment for MF in at least four weeks receive (i) an initial loading dose of PRM-151 at 10 mg/kg by intravenous infusion on days 1, 3, and 5 of cycle 1 (a 28-day cycle), and (ii) thereafter were administered a dose of PRM-151 at 10 mg/kg by intravenous infusion on day 1 of each subsequent 28-day cycle for nine cycles. In certain embodiments, the randomization is stratified according to type of subject (subjects with Hgb <100 g/L and having received ≥2 units PRBC in the 12 weeks prior to study entry OR subjects with platelet count <50×10⁹/L) and ensures that the final study population will include at least 50% of subjects from the second stratum (platelet count <50×10⁹/L). All subjects may switch to an open label extension and receive PRM-151 10 mg/kg every 4 weeks completing 9 cycles of the originally assigned treatment. After study completion and data analysis, all subjects remaining on PRM-151 switch to the dose that has been selected for future development based on study results. Enrolled subjects are considered evaluable for response if they are on study drug for at least twelve weeks.

Patients in each cohort are monitored for improvements in bone marrow fibrosis (BMF) (by quantitative image analysis, for example) and disease related anemia, thrombocytopenia, peripheral blood blasts, constitutional symptoms, and spleen size. The IWG-MRT response (complete response, partial response, clinical improvement), and the effect on PRM-151 on the rate of stable and progressive disease is assessed. Further, patients are monitored for changes in other disease related hematologic abnormalities, changes in prognostic factors associated with increased mortality as measured by the DIPSS (Dynamic International Prognostic Scoring System), changes in bone marrow morphology, changes in bone marrow metabolism as measured by PET imaging. In addition, the interaction between genetic mutations and cytogenetic abnormalities and response to PRM-151 is evaluated and biomarkers of PRM-151 activity in bone marrow samples are evaluated. Correlation between baseline SAP levels and patient outcomes are assessed. The relationship between bone marrow fibrosis reduction and hematologic improvements is assessed. Patients are also monitored for overall response rate according to the International Working Group consensus criteria for treatment response in myelofibrosis with myeloid metaplasia (Tefferi A, Cervantes F. Mesa R, et al. Revised response criteria for myelofibrosis: International Working Group-Mycloproliferative Neoplasms Research and Treatment (IWG-MRT) and European LeukemiaNet (ELN) consensus report. Blood. 2013; 122:1395-1398). Patients are also monitored for incidence of adverse events, changes in bone marrow fibrosis by WHO criteria as described in the European Consensus of Grading Bone Marrow Fibrosis (Thiele J, Kvasnicka H M, Facchetti F, et al. European consensus on grading bone marrow fibrosis and assessment of cellularity. Haematologica 2005; 90:1128-1132.), changes in the modified Myeloproliferative Neoplasma Symptom Assessment Form (MPN-SAF) Score (Emanuel et al. 2012, Journal of Clinical Oncology 30(33): 4098-4103), and changes in quality of life as measured by the EORTC QLQ-C30 score (EORTC QLQ-C30 (version 3) 1995, EORTC Quality of Life Group). Progression-free and overall survival is measured.

Optionally, the patients in each cohort are monitored for one or more of the effects described herein, for example, the effect of the treatment on a reduction in bone marrow fibrosis score by at least one grade according to WHO criteria is evaluated as determined by a central adjudication panel of expert hematopathologists, blinded to subject, treatment, and time of biopsy. Optionally, the effect of the treatment on hematologic improvements such as RBC transfusion independence, platelet transfusion dependence, or a 10-20 g/L increase in hemoglobin levels is further monitored.

Mutational status and allele burden is optionally evaluated prior to initiation of treatment. Allele burden is optionally evaluated following initiation of treatment, and may be evaluated multiple times over the course of treatment (e.g., following 1, 2, 3, 4, 5 or 6 cycles). At time points throughout the study, blood samples are collected and DNA is isolated from peripheral whole blood for the purpose of associating baseline mutational status with select primary and secondary endpoints. Samples are analyzed for mutational status of JAK2, MPL, CALR, ASXL1, EZH2, SRSF2, IDH1, and/or IDH2. Samples are also analyzed to assess changes in allele burden of JAK2V617F at, for example, week 36 (Cycle 1 Day 1 to Cycle 9 Day 29). Samples are also analyzed to assess changes in the allele burden of MPLW515, CALR, ASXL1, EZH2, SRSF2, IDH1, and/or IDH2 at, for example, week 36.

Immunohistochemical analysis of additional bone marrow biopsy samples for disease and mechanism related proteins and cellular markers is optionally performed.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will be apparent to those skilled in the art upon review of this specification and the below-listed claims. The full scope of the disclosure should be determined by reference to the claims, along with their full scope of equivalents, and the specification, along with such variations.

### SEQUENCE LISTING

| SEQ ID NO: 1 human serum amyloid protein P | |
|---|---|
| HTDLSGKVFVFPRESVTDHVNLITPLEKPLQNFTLCFRAYSDLSRAYSLFSYNTQGRD | |
| NELLVYKERVGEYSLYIGRHKVTSKVIEKFPAPVHICVSWESSSGIAEFWINGTPLVK | |
| KGLRQGYFVEAQPKIVLGQEQDSYGGKFDRSQSFVGEIGDLYMWDSVLPPENILSAY | |
| QGTPLPANILDWQALNYEIRGYVIIKPLVWV | |
| SEQ ID NO: 2 Gallus gallus serum amyloid protein P | |
| QEDLYRKVFVFREDPSDAYVLLQVQLERPLLNFTVCLRSYTDLTRPHSLFSYATKAQ | |
| DNEILLFKPKPGEYRFYVGGKYVTFRVPENRGEWEHVCASWESGSGIAEFWLNGRP | |
| WPRKGLQKGYEVGNEAVVMLGQEQDAYGGGFDVYNSFTGEMADVHLWDAGLSP | |
| DKMRSAYLALRLPPAPLAWGRLRYEAKGDVVVKPRLREALGA | |
| SEQ ID NO: 3 Bos taurus serum amyloid protein | |
| QTDLRGKVFVFPRESSTDHVTLITKLEKPLKNLTLCLRAYSDLSRGYSLFSYNIHSKD | |
| NELLVFKNGIGEYSLYIGKTKVTVRATEKFPSPVHICTSWESSTGIAEFWINGKPLVKR | |
| GLKQGYAVGAHPKIVLGQEQDSYGGGFDKNQSFMGEIGDLYMWDSVLSPEEILLVY | |
| QGSSSISPTILDWQALKYEIKGYVIVKPMVWG | |
| SEQ ID NO: 4 Cricetulus migratorius serum amyloid protein P | |
| QTDLTGKVFVFPRESESDYVKLIPRLEKPLENFTLCFRTYTDLSRPHSLFSYNTKNKD | |
| NELLIYKERMGEYGLYIENVGAIVRGVEEFASPVHFCTSWESSSGIADFWVNGIPWV | |
| KKGLKKGYTVKTQPSIILGQEQDNYGGGFDKSQSFVGEMGDLNMWDSVLTPEEIKS | |
| VYEGSWLEPNILDWRALNYEMSGYAVIRPRVWH | |
| NM_004972 Homo sapiens Janus kinase 2 (JAK2), mRNA | |
| (SEQ ID NO: 5) | |
| 1 | ctgcaggaag gagagaggaa gaggagcaga agggggcagc agcggacgcc gctaacggcc |
| 61 | tccctcggcg ctgacaggct gggccggcgc ccggctcgct tgggtgttcg cgtcgccact |
| 121 | tcggcttctc ggccggtcgg gcccctcggc ccgggcttgc ggcgcgcgtc ggggctgagg |
| 181 | gctgctgcgg cgcagggaga ggcctggtcc tcgctgccga gggatgtgag tgggagctga |
| 241 | gcccacactg gagggccccc gagggcccag cctggaggtc gttcagagcc gtgcccgtcc |
| 301 | cggggcttcg cagaccttga cccgccgggt aggagccgcc cctgcgggct cgagggcgcg |
| 361 | ctctggtcgc ccgatctgtg tagccggttt cagaagcagg caacaggaac aagatgtgaa |
| 421 | ctgtttctct tctgcagaaa aagaggctct tcctcctcct cccgcgacgg caaatgttct |
| 481 | gaaaaagact ctgcatggga atggcctgcc ttacgatgac agaaatggag ggaacatcca |
| 541 | cctcttctat atatcagaat ggtgatattt ctggaaatgc caattctatg aagcaaatag |
| 601 | atccagttct tcaggtgtat ctttaccatt cccttgtgaa atctgaggca gattatctga |
| 661 | cctttccatc tggggagtat gttgcagaag aaatctgtat tgctgcttct aaagcttgtg |
| 721 | gtatcacacc tgtgtatcat aatatgtttg ctttaatgag tgaaacagaa aggatctggt |
| 781 | atccacccaa ccatgtcttc catatagatg agtcaaccag gcataatgta ctctacagaa |
| 841 | taagatttta ctttcctcgt tggtattgca gtggcagcaa cagagcctat cggcatggaa |
| 901 | tatctcgagg tgctgaagct cctcttcttg atgactttgt catgtcttac ctctttgctc |
| 961 | agtggcggca tgattttgtg cacggatgga taaaagtacc tgtgactcat gaaacacagg |
| 1021 | aagaatgtct tgggatggca gtgttagata tgatgagaat agccaaagaa aacgatcaaa |
| 1081 | ccccactggc catctataac tctatcagct acaagacatt cttaccaaaa tgtattcgag |
| 1141 | caaagatcca agactatcat attttgacaa ggaagcgaat aaggtacaga tttcgcagat |
| 1201 | ttattcagca attcagccaa tgcaaagcca ctgccagaaa cttgaaactt aagtatctta |
| 1261 | taaatctgga aactctgcag tctgccttct acacagagaa atttgaagta aaagaacctg |
| 1321 | gaagtggtcc ttcaggtgag gagatttttg caaccattat aataactgga aacggtggaa |
| 1381 | ttcagtggtc aagagggaaa cataaagaaa gtgagacact gacagaacag gatttacagt |
| 1441 | tatattgcga ttttcctaat attattgatg tcagtattaa gcaagcaaac caagagggtt |
| 1501 | caaatgaaag ccgagttgta actatccata agcaagatgg taaaaatctg gaaattgaac |
| 1561 | ttagctcatt aagggaagct ttgtctttcg tgtcattaat tgatggatat tatagattaa |
| 1621 | ctgcagatgc acatcattac ctctgtaaag aagtagcacc tccagccgtg cttgaaaata |
| 1681 | tacaaagcaa ctgtcatggc ccaatttcga tggattttgc cattagtaaa ctgaagaaag |
| 1741 | caggtaatca gactggactg tatgtacttc gatgcagtcc taaggacttt aataaatatt |
| 1801 | ttttgacttt tgctgtcgag cgagaaaatg tcattgaata taaacactgt ttgattacaa |
| 1861 | aaaatgagaa tgaagagtac aacctcagtg ggacaaagaa gaacttcagc agtcttaaag |
| 1921 | atcttttgaa ttgttaccag atggaaactg ttcgctcaga caatataatt ttccagttta |
| 1981 | ctaaatgctg tcccccaaag ccaaaagata aatcaaacct tctagtcttc agaacgaatg |
| 2041 | gtgtttctga tgtaccaacc tcaccaacat tacagaggcc tactcatatg aaccaaatgg |
| 2101 | tgtttcacaa aatcagaaat gaagatttga tatttaatga aagccttggc caaggcactt |
| 2161 | ttacaaagat ttttaaaggc gtacgaagag aagtaggaga ctacggtcaa ctgcatgaaa |
| 2221 | cagaagttct tttaaaagtt ctggataaag cacacagaaa ctattcagag tctttctttg |
| 2281 | aagcagcaag tatgatgagc aagctttctc acaagcattt ggttttaaat tatggagtat |
| 2341 | gtgtctgtgg agacgagaat attctggttc aggagtttgt aaaatttgga tcactagata |
| 2401 | catatctgaa aaagaataaa aattgtataa atatattatg gaaacttgaa gttgctaaac |
| 2461 | agttggcatg ggccatgcat tttctagaag aaaacaccct tattcatggg aatgtatgtg |
| 2521 | ccaaaaatat tctgcttatc agagaagaag acaggaagac aggaaatcct cctttcatca |
| 2581 | aacttagtga tcctggcatt agtattacag ttttgccaaa ggacattctt caggagagaa |
| 2641 | taccatgggt accacctgaa tgcattgaaa atcctaaaaa tttaaatttg gcaacagaca |
| 2701 | aatggagttt tggtaccact ttgtgggaaa tctgcagtgg aggagataaa cctctaagtg |
| 2761 | ctctggattc tcaaagaaag ctacaatttt atgaagatag gcatcagctt cctgcaccaa |
| 2821 | agtgggcaga attagcaaac cttataaata attgtatgga ttatgaacca gatttcaggc |
| 2881 | cttctttcag agccatcata cgagatctta acagtttgtt tactccagat tatgaactat |
| 2941 | taacagaaaa tgacatgtta ccaaatatga ggataggtgc cctggggttt tctggtgcct |
| 3001 | ttgaagaccg ggatcctaca cagtttgaag agagacattt gaaatttcta cagcaacttg |
| 3061 | gcaagggtaa ttttgggagt gtggagatgt gccggtatga ccctctacag gacaacactg |
| 3121 | gggaggtggt cgctgtaaaa aagcttcagc atagtactga agagcaccta agagactttg |
| 3181 | aaagggaaat tgaaatcctg aaatccctac agcatgacaa cattgtaaag tacaagggag |
| 3241 | tgtgctacag tgctggtcgg cgtaatctaa aattaattat ggaatattta ccatatggaa |
| 3301 | gtttacgaga ctatcttcaa aaacataaag aacggataga tcacataaaa cttctgcagt |
| 3361 | acacatctca gatatgcaag ggtatggagt atcttggtac aaaaaggtat atccacaggg |
| 3421 | atctggcaac gagaaatata ttggtggaga acgagaacag agttaaaatt ggagattttg |
| 3481 | ggttaaccaa agtcttgcca caagacaaag aatactataa agtaaaagaa cctggtgaaa |
| 3541 | gtcccatatt ctggtatgct ccagaatcac tgacagagag caagttttct gtggcctcag |
| 3601 | atgtttggag ctttggagtg gttctgtatg aacttttcac atacattgag aagagtaaaa |
| 3661 | gtccaccagc ggaatttatg cgtatgattg gcaatgacaa acaaggacag atgatcgtgt |
| 3721 | tccatttgat agaacttttg aagaataatg gaagattacc aagaccagat ggatgcccag |
| 3781 | atgagatcta tatgatcatg acagaatgct ggaacaataa tgtaaatcaa cgcccctcct |
| 3841 | ttagggatct agctcttcga gtggatcaaa taagggataa catggctgga tgaaagaaat |
| 3901 | gaccttcatt ctgagaccaa agtagattta cagaacaaag ttttatattt cacattgctg |
| 3961 | tggactatta ttacatatat cattattata taaatcatga tgctagccag caaagatgtg |
| 4021 | aaaatatctg ctcaaaactt tcaaagttta gtaagttttt cttcatgagg ccaccagtaa |
| 4081 | aagacattaa tgagaattcc ttagcaagga ttttgtaaga agtttcttaa acattgtcag |
| 4141 | ttaacatcac tcttgtctgg caaaagaaaa aaaatagact ttttcaactc agctttttga |
| 4201 | gacctgaaaa aattattatg taaattttgc aatgttaaag atgcacagaa tatgtatgta |
| 4261 | tagtttttac cacagtggat gtataatacc ttggcatctt gtgtgatgtt ttacacacat |
| 4321 | gagggctggt gttcattaat actgttttct aatttttcca tagttaatct ataattaatt |
| 4381 | acttcactat acaaacaaat taagatgttc agataattga ataagtacct ttgtgtcctt |
| 4441 | gttcatttat atcgctggcc agcattataa gcaggtgtat acttttagct tgtagttcca |
| 4501 | tgtactgtaa atatttttca cataaaggga acaaatgtct agttttattt gtataggaaa |
| 4551 | tttccctgac cctaaataat acattttgaa atgaaacaag cttacaaaga tataatctat |
| 4621 | tttattatgg tttcccttgt atctatttgt ggtgaatgtg ttttttaaat ggaactatct |
| 4681 | ccaaattttt ctaagactac tatgaacagt tttcttttaa aattttgaga ttaagaatgc |
| 4741 | caggaatatt gtcatccttt gagctgctga ctgccaataa cattcttcga tctctgggat |
| 4801 | ttatgctcat gaactaaatt taagcttaag ccataaaata gattagattg ttttttaaaa |
| 4861 | atggatagct cattaagaag tgcagcaggt taagaatttt ttcctaaaga ctgtatattt |
| 4921 | gaggggtttc agaattttgc attgcagtca tagaagagat ttatttcctt tttagagggg |
| 4981 | aaatgaggta aataagtaaa aaagtatgct tgttaatttt attcaagaat gccagtagaa |
| 5041 | aattcataac gtgtatcttt aagaaaaatg agcatacatc ttaaatcttt tcaattaagt |
| 5101 | ataaggggtt gttcgttgtt gtdatttgtt atagtgctac tccactttag acaccatagc |
| 5161 | taaaataaaa tatggtgggt tttgtgtgtg tgtgtgtgtg tgtgtgtgtg tgtgtgtgtg |
| 5221 | tgttatttat acaaaactta aaatacttgc tgttttgatt aaaaagaaaa tagtttctta |
| 5281 | cttt |
| NP_004963 tyrosine-protein kinase JAK2 [Homo sapiens] | |
| (SEQ ID NO: 6) | |
| 1 | mgmacltmte megtstssiy qngdisgnan smkqidpvlq vylyhslgks eadyltfpsg |
| 61 | eyvaeeicia askacgitpv yhnmfalmse teriwyppnh vfhidestrh nvlyrirfyf |
| 121 | prwycsgsnr ayrhgisrga eapllddfvm sylfaqwrhd fvhgwikvpv thetqeeclg |
| 181 | mavldmmria kendqtplai ynsisyktfl pkcirakiqd yhlltrkrir yrfrrfiqqf |
| 241 | sqckatarnl klkylinlet lqsafytekf evkedgsgps geeifatiii tgnggiqwsr |
| 301 | gkhkesetlt eqdlqlycdf pniidvsikq anqegsnesr vvtlhkqdgk nleielsslr |
| 361 | ealsfvslid gyyrltadah hylckevapp avleniqsnc hgpismdfai sklkkagnqt |
| 421 | glyvlrcspk dfnkyfltfa verenvieyk hclitknene eynlsgtkkn fsslkdllnc |
| 481 | yqmetvrsdn iitqftkccp pkpkdksnll virtngvsdv ptsptlqrpt hmnqmvthki |
| 541 | rnedlifnes lgqgtftkif kgvrrevgdy gqlhetevll kvldkahrny sesffeaasm |
| 601 | msklshkhlv lnygvcvcgd enilvqefvk fgsldtylkk nkncinilwk levakqlawa |
| 561 | mhfleentli hgnvcaknil lireedrktg nppfiklsdp gisitvlpkd ilqeripwvp |
| 721 | pecienpknl nlatdkwsfg ttlweicsgg dkplsaldsq rklqfyedrh qlpapkwael |
| 781 | anlinncmdy epdfrpsfra iirdlnslft pdyelltend mlpnmrigal gfsgafedrd |
| 841 | ptqfeerhlk flqqigkgnf gsvemcrydp lqdntgevva vkklqhstee hlrdfereie |
| 901 | ilkslqhdni vkykgvdysa grrnlklime ylpygslrdy lqkhkeridh ikllqytsqi |
| 961 | ckgmeylgtk ryihrdlatr nilvenenrv kigdfgltkv lpqdkeyykv kepgespifw |
| 1021 | yapesltesk fsvasdvwsf gvvlyelfty ieksksppae fmrmigndkq qqmivfhlie |
| 1081 | llknngrlpr pdgcpdeiym imtecwnnnv nqrpsfrdla lrvdqirdnm ag |
| (SEQ ID NO: 7) NM_005373 Homo sapiens MPL proto-oncogene, thrombopoietin receptor | |
| (MPL), mRNA | |
| 1 | cctgaaggga ggatgggcta aggcaggcac acagtggcgg agaagatgcc ctcctgggcc |
| 61 | ctcttcatgg tcacctcctg cctcctcctg gcccctcaaa acctggccca agtcagcagc |
| 121 | caagatgtct ccttgctggc atcagactca gagcccctga agtgtttctc ccgaacattt |
| 181 | gaggacctca cttgcttctg ggatgaggaa gaggcagccc ccagtgggac ataccagctg |
| 241 | ctgtatgcct acccgcggga gaagccccgt gcttgccccc tgagttccca gagcatgccc |
| 301 | cactttggaa cccgatacgt gtgccagttt ccagaccagg aggaagtgcg tctcttcttt |
| 361 | ccgctgcacc tctgggtgaa gaatgtgttc ctaaaccaga ctcggactca gcgagtcctc |
| 421 | tttgtggaca gtgtaggcct gccggctccc cccagtatca tcaaggccat gggtgggagc |
| 481 | cagccagggg aacttcagat cagctgggag gagccagctc cagaaatcag tgatttcctg |
| 541 | aggtacgaac tccgctatgg ccccagagat cccaagaact ccactggtcc cacggtcata |
| 601 | cagctgattg ccacagaaac ctgctgccct gctctgcaga ggcctcactc aucctctgct |
| 661 | ctggaccagt ctccatgtgc tcagcccaca atgccctggc aagatggacc aaagcagacc |
| 721 | tccccaagta gagaagcttc agctctgaca gcagagggtg gaagctgcct catctcagga |
| 781 | ctccagcctg gcaactccta ctggctgcag ctgcgcagcg aacctgatgg gatctccctc |
| 841 | ggtggctcct ggggatcctg gtccctccct gtgactgtgg acctgcctgg agatgcagtg |
| 901 | gcacttggac tgcaatgctt taccttggac ctgaagaatg ttacctgtca atggcagcaa |
| 961 | caggaccatg ctagctccca aggcttcttc taccacagca gggcacggtg ctgccccaga |
| 1021 | gacaggtacc ccatctggga gaactgcgaa gaggaagaga aaacaaatcc aggactacag |
| 1081 | accccacagt tctctcgctg ccacttcaag ttacgaaatg acagcattat tcacatcctt |
| 1141 | gtggaggtga ccacagcccc gggtactgtt cacagctacc tgggctcccc tttctggatc |
| 1201 | caccaggctg tgcgcctccc caccccaaac ttgcactgga gggagatctc cagtgggcat |
| 1261 | ctggaattgg agtggcagca cccatcgtcc tgggcagccc aagagacctg ttatcaactc |
| 1321 | cgatacacag gagaaggcca tcaggactgg aaggtgctgg agccgcctct cggggcccga |
| 1381 | ggagggaccc tggagctgcg cccgcgatct cgctaccgtt tacagctgcg cgccaggctc |
| 1441 | aacggcccca cctaccaagg tccctggagc tcgtggtcgg acccaactag ggtggagacc |
| 1501 | gccaccgaga ccgcctggat cfccttggtg accgctctgc atctagtgct gggcctcagc |
| 1561 | gccutcctgg gcctgctgct gctgaggtgg cagtttcctg cacactacag gagactgagg |
| 1621 | catgccctgt ggccctcact tccagacctg caccgggtcc taggccagta ccttagggac |
| 1681 | actgcagccc tgagcccgcc caaggccaca gtctcagata cctgtgaaga agtggaaccc |
| 1741 | agcctccttg aaatcctccc caagtcctca gagaggactc ctttgcccct gtgttcctcc |
| 1801 | caggcccaga tggactaccg aagattgcag ccttcttgcc tggggaccat gcccctgtct |
| 1861 | gtgtgcccac ccatggctga gtcagggtcc tgctgtacca cccacattgc caaccattcc |
| 1921 | tacctaccac taagctattg gcagcagcct tgaggacagg ctcctcactc ccagttccct |
| 1981 | ggacagagct aaactctcga gacttctctg tgaacttccc taccctaccc ccacaacaca |
| 2041 | agcaccccag acctcacctc cattcccctc tgtctgccct cacaattagg cttcattgca |
| 2101 | ctgatcttac tctactgctg ctgacataaa accaggaccc tttctccaca ggcaggctca |
| 2161 | tttcactaag ctcctccttt actttctctc tcctctttga tctcaaacgc cttgaaaaca |
| 2221 | agcctccact tccccacact tcccatttac tcttgagact acttcaatta gttcccctac |
| 2281 | tacactttgc tagtgaaact gcccaggcaa agtgcaccic aaatcttcta attccaagat |
| 2341 | ccaataggat ctcgttaatc atcagttcct ttgatctcgc tgtaagattt gtcaaggctg |
| 2401 | actactcact tctcctttaa attctttcct accttggtcc tgcctctttg agtatattag |
| 2461 | tagctttttt ttatttgttt gagacagggt ctcactctgt cacccaggct gcagtgcaat |
| 2521 | ggcgcgatct cagctcactg caacctccac ctccgggttc aagcgattct tgtgcctcgg |
| 2581 | cctccctagt agctgggatt acaggcgcac accaccacac acagctaatt ttattttttt |
| 2641 | tttttttttt ttttttttag acggagcctt gctctgttgc cagactggag tgcagtggca |
| 2701 | cgatctcggc tcactgcaac ctctgcctcc cgggttcaag ccattctgcc tcagcctccc |
| 2761 | aagtagctgg gagtacaggc gtctgccacc atgcctaatt tttttctatt tttaggagag |
| 2821 | accggttttc accacgttgg ccaggatggt ctcgatatcc tgatctcgtg atccgcctgc |
| 2881 | ctctgcctcc caaagtgctg ggattacagg tgtgacccac tgcgcacagc cccagctaat |
| 2941 | tttcatattt ttagtagaga cagggttttg ccatgttgcc caggctggtc ttgaactcct |
| 3001 | aacctcgggt gatccaccca ccttggcctc ccaaagtgtt aggattacag gcatgagcca |
| 3061 | ctgcgcccgg ctgagtctac tagtagttaa gagaataaac tagatctaga atcagagctg |
| 3121 | gattcaattc ctgtccttca catttactag ctgtgcaacc ttgggcacat aacttaatgt |
| 3181 | ctttgagcct tagttatttc atctgtaaaa cagggataat aacagcaccc catagagttg |
| 3241 | tgacgaggat tgagataatc taagtaaagc acagtcccta ggacatagta aatgattcat |
| 3301 | atatccgaac tactgttata attattcctt cttactctcc tcttctagca tttcttccaa |
| 3361 | ttattacagt ccttcaagat tccatttctt aacagtctcc catcccatct attctctgcc |
| 3421 | tttactatat gttgaccatt ccaaagttct tatctctagc tcagacatct actacagcac |
| 3481 | tgtgatgctt tatgcaacta actgtttaca tatctgtccc ctgctactag attgtgagct |
| 3541 | ccttgaggga aaggaacatg atttatttgt ccttttcccc cagcacctag agtagtgctt |
| 3601 | ggtgcatgat agtaggcctt caatcaattt tttctaactg aatga |
| (SEQ ID NO: 8) NM__005364 thrombopoietin receptor precursor [Homo sapiens] | |
| 1 | mpswalfvmt sclllapqnl aqvssqdvsl lasdseplkc fsrtfedltc fwdeeeaaps |
| 61 | gtyqllyayp rekpracpls sqsmphfgtr yvcqgpdqee vrlffplhlw vknvflnqtr |
| 121 | tqrvlfvdsv glpappsiik amggsqpgel qisweepape isdflryelr ygprdpknst |
| 181 | gptviqliat etccpalqrp hsasaldqsp caqptmpwqd gpkqtspsre asaltaeggs |
| 241 | clisglqpgn sywlqlrsep dgislggswg swslpvtvdl pgdavalglq ctfldlknvt |
| 301 | cqwqqqdhas sqgffyhsra rccprdrypi wenceeeekt npglqtpgfs rchfksrnds |
| 361 | iihilvevtt apgtvhsylg spfwihqavr lptpnlhwre issghlelew qhpsswaaqe |
| 421 | tcyqlrytge ghqdwkvlep plgarggtle lrprsryrlq lrarlngpty qgpwsswsdp |
| 481 | trvetateta wislvtalhl vlglsavlgl lllrwqfpah yrrlrhalwp slpdlhrvlg |
| 541 | qlyrdtaals ppkatvsdtc eevepsllei lpkssertpl plcssqaqmd yrrlqpsclg |
| 601 | tmplsvcppm aesgscctth ianhsylpls ywqqp |
| (SEQ ID NO: 9) NM_004343 Homo sapiens calreticulin (CALR), mRNA | |
| 1 | gcggcgtccg tccgtactgc agagccgctg ccggagggtc gttttaaagg gcccgcgcgt |
| 61 | tgacgccccc tcggcccgcc atgctgctat ccgtgccgct gctgctcggc ctcctcggcc |
| 121 | tggccgtcgc cgagcctgac gtctacttca aggagcagtt tctggacgga gacgggtgga |
| 181 | cttcacactg gatcgaatcc aaacacaagt cagattttgg caaattcgtt ctaagttccg |
| 241 | gcaagttcta cggtgacgag gagaaagata aaggtttgca gacaagccag gatgcacgct |
| 301 | tttatgctct gtcggccagt ttcgagcctt tcagcaacaa aggccagacg ctggtggtga |
| 361 | agttcacggt gaaacatgag cagaacatcg actgtggggg cggctatgtg aagctgtttc |
| 421 | ctaatagttt ggaccagaca gacatgcacg gagactcaga atacaacatc atgtttggtc |
| 481 | ccgacatctg tggccctggc accaagaagg ttcatgtcat cttcaactac aagggcaaga |
| 541 | acgtgctgat caacaaggac atccgttgca aggatgatga gtttacacac ctgtacacac |
| 601 | tgattgtgcg gccagacaac acctatgagg tgaagattga caacagccag gtggagtacg |
| 661 | gctccttgga agacgattgg gacttcctgc cacccaagaa gataaaggat cctgatgctt |
| 721 | caaaaccgga agactgggat gagcgggcca agatcgatga tcccacagac tccaagcctg |
| 781 | aggactggga caagcacgag catatccctg accctgatgc taagaagccc gaggactggg |
| 841 | atgaagagat ggacggagag tgggaacccc cagtgattca gaaccctgag tacaagggtg |
| 901 | agtggaagcc ccggcagatc gacaacccag attacaaggg cacttggatc cacccagaaa |
| 961 | ttgacaaccc cgagtattct cccgatccca gtatctatgc ctatgataac tttggcgtgc |
| 1021 | tgggcctgga cctctggcag gtcaagtctg gcaccatctt tgacaacttc ctcatcacca |
| 1081 | acgatgaggc atacgctgag gagtttggca acgagacgtg gggcgtaaca aaggcagcag |
| 1141 | agaaacaaat gaaggacaaa caggacgagg agcagaggct taaggaggag gaagaagaca |
| 1201 | agaaacgcaa agaggaggag gaggcagagg acaaggagga tgatgaggac aaagatgagg |
| 1261 | atgaggagga tgaggaggac aaggaggaag atgaggagga agatgtcccc ggccaggcca |
| 1321 | aggacgagct gtagagaggc ctgcctccag ggctggactg aggcatgagc gctcctgccg |
| 1381 | cagagctggc cgcgccaaat aatgtatctg tgagactcga gaactttcat ttttttccag |
| 1441 | gctggttcgg atttggggtg gattttggtt ttgttcccct cctccactct cccccacccc |
| 1501 | ctccccgccc tttttttttt ttttttttaa actggtattt tatctttgat tctccttcag |
| 1561 | ccatcacccc tggttctcat ctttcttgat caacatcttt tcttgcctct gtccccttct |
| 1621 | ctcatctctt agctcccctc caacctgggg ggcagtggtg tggagaagcc acaggcctga |
| 1681 | gatttcatct gctctccttc ctggagccca gaggagggca gcagaagggg gtggtgtatc |
| 1741 | caacccacca gaactgagga agaacggggc tcttctcctt tcacccatcc ctttctcccc |
| 1801 | tgcccccagg actgggccac ttctgggtgg ggcagtgggt cccagattgg ctcacactga |
| 1861 | gaatgtaaga actacaaaca aaatttctat taaattaaat tttgtgtctc caaaaaaaaa |
| 1921 | aaaaaaaaa |
| (SEQ ID NO: 10) NM_004334 calreticulin precursor [Homo sapiens] | |
| 1 | mllsvplllg llglavaepa vyfkeqfldg dgwtsrwies khksdfgkfv lssgkfygde |
| 61 | ekdkglqtsq darfyalsas fepfsnkgqt lvvqftvkhe qnidcgggyv klfpnsldqt |
| 121 | dmhgdseyni mfgpdicgpg tkkvhvifny kgknvlinkd irckddefth lytlivrpdn |
| 181 | tyevkidnsq vesgsleddw dflppkkikd pdaskpedwd erakiddptd skpedwdkpe |
| 241 | hipdpdakkp edwdeemdge weppviqnpe ykgewkprqi dnpdykgtwi hpeidnpeys |
| 301 | pdpsiyaydn fgvlgldlwq vksgtifdnf litndeayae efgnetwgvt kaaekqmkdk |
| 361 | qdeeqrlkee eedkkrkeee eaedkedded kdedeedeed keedeeedvp gqakdel |
| (SEQ ID NO: 11) NM_001164603 Homo sapiens additional sex combs like transcriptional | |
| regulator 1(ASXL1), transcript variant 2, mRNA | |
| 1 | cacacccacg gcagacacgc acgcacccgg gcgccgaagg gaaagccgcg tctcgccctc |
| 61 | ccgccccgcc gtcggtcctg tctcagtcpc tcagcagagc gggaaagcgg aggccggagc |
| 121 | cgtgacctct gaccccgtgg ttatgcggag ccgccgcatt ccttagcgat cgcggggcag |
| 181 | ccgccgctgc cgccgtgggc gactgacgca gcgcgggcgc gtggagccgc cgccgcccct |
| 241 | cccccaccgc cgctctcgcg ccagccggtc cccgcgtgcc cgccccttct ccccggccgc |
| 301 | acccgagacc tcgcgcgccg ccgctgccac gcgccccccc caccgccgcc gccgccccag |
| 361 | ccccgcgcca ccgccccagc ccgcccagcc cggaggtccc gcgtggagct gccgccgccg |
| 421 | ccggggagaa ggatgaagga caaacagaag aagaagaagg agcgcacgtg ggccgaggcc |
| 481 | gcgcgcctgg tattagadaa ctactcggat gctccaatga caccaaaaca gattctgcag |
| 541 | gtcatagagg cagaaggact aaaggaaatg agaagtggga cttcccctct cgcatgcctc |
| 601 | aatgctatgc tacattccaa ttcaagagga ggagaggggt tgttttataa actgcctggc |
| 661 | cgaatcagcc ttttcacgct caaggtgtga gccactgcac caggcccctt catcttaatt |
| 721 | ttaatatatc tttgaataaa caccattgta tgaacctgct gtaagcttgg gagtggtctg |
| 781 | ttagtctaca gcttgtgtct gagatgtgct aattgaatat ttgctcagta cctcatctta |
| 841 | actgcctttg gctttatgtt gcttatcctt catagtatct tgttcattgg ccttttacat |
| 901 | ccataggcat cacttctctg atattcgttg tgctacttta atggattaat ggtttgcttg |
| 961 | gtaggttcct ctagttagac tgtaaactcc ttgagagcag agtctgtatt ttattaatta |
| 1021 | cccacagtcc taggtacata gttgccttca atcaatatat atttaatgaa aaaaaaaaaa |
| 1081 | aaaa |
| (SEQ ID NO: 12) NP_001158075 putative Polycomb group protein ASXL1 isoform2 [Homo | |
| sapiens] | |
| 1 | mkdkqkkkke rtwaeaarlv lenysdapmt pkqilqviea eglkemrsgt splaclnaml |
| 61 | hsnsrggegl fyklpgrisl ftlkv |
| (SEQ ID NO: 13) NM_001203247 Homo sapiens enhancer of zeste homolog 2 (Drosophila) | |
| (EZH2), transcript variant 3, mRNA | |
| 1 | ggcggcgctt gattgggctg ggggggccaa ataaaagcga tggcgattgg gctgccgcgt |
| 61 | ttggcgctcg gtccggtcgc gtccgacacc cggtgggact cagaaggcag tggagccccg |
| 121 | gcggcggcgg cggcggcgcg cgggggcgac gcgcgggaac aacgcgagtc ggcgcgcggg |
| 181 | acgaagaata atcatgggcc agactgggaa gaaatctgag aagggaccag tttgttggcg |
| 241 | gaagcgtgta aaatcagagt acatgcgact gagacagctc aagaggttca gacgagctga |
| 301 | tgaagtaaag agtatgttta gttccaatcg tcagaaaatt ttggaaagaa cggaaatctt |
| 361 | aaaccaagaa tggaaacagc gaaggataca gcctgtgcac atcctgactt ctgtgagctc |
| 421 | attgcgcggg actagggagt gttcggtgac cagtgacttg gattttcaaa cacaagtcat |
| 481 | cccattaaag actctgaatg cagttgcttc agtacccata atgtattctt ggtctcccct |
| 541 | acagcagaat tttatggtgg aagatgaaac tgttttacat aacattcctt atatgggaga |
| 601 | tgaagtttta gatcaggatg gtactttcat tgaagaacta ataaaaaatt atgatgggaa |
| 661 | agtacacggg gatagagaat gtgggtttat aaatgatgaa atttttgtgg agtaggtgaa |
| 721 | tgcccttggt caatataatg atgatgacga tgatgatgat cmagacgatc ctgaagaaag |
| 781 | agaagaaaag cagaaagatc tggaggatca ccgagatgat aaagaaagcc gcccacctcg |
| 341 | gaaatttcct tctgataaaa tttttgaegc catttcctca atgtttccag ataagggcac |
| 901 | agcagaagaa ctaaaggaaa aatataaaga actcaccgaa cagaagctcc caggcgaact |
| 961 | tcctcctgaa tgtaccccca acatagatgg accaaatgct aaatctgttc agagagagca |
| 1021 | aagcttacac tcctttcata cgcttttctg taggcgatgt tttaaatatg actgcttcct |
| 1081 | acatcctttt catgcaacac ccaacactta taagcggaag aacacagaaa cagctctaga |
| 1141 | caacaaacct tgtggaccac agtgttacca gcatttggag guagcaaagg agtttgctgc |
| 1201 | tgctctcacc gatgagcgga taaagacccc accaaaacgt ccaggaggcc gcagaagagg |
| 1261 | acggcttccc aataacagta gcaggcccag cacccccacc attaatgtgc tggaatcaaa |
| 1321 | ggatacagac agtgataggg aagcagggac tgaaacgggg ggagagaaca atgataaaga |
| 1381 | agaagaagag aagaaagatg aaacttcgag ctcctctgaa gcaaattctc ggtgtcaaac |
| 1441 | accaataaag atgaagccaa atattgaacc tcctgagaat gtggagtgga gtggtgctga |
| 1501 | agcctcaatg tttagagtcc tcattggcac ttactatgac aatttctgtg ccattgatag |
| 1561 | gttaattggg acaaaaacat gtagacaggt gtatgagttt agagtcaaag aatctagcat |
| 1621 | catagctcca gctcccgctg aggatgtgga tactcctcca aggaaaaaga agaggaaaca |
| 1681 | ccggttgtgg gctgcacact gcagaaagat acagctgaaa aaggacggct cctctaacca |
| 1741 | tgtttacaac tatcaaccct gtgatcatcc acggcagcct tgtgacagtt cgtgcccttg |
| 1801 | tgtgatagca caaaattttt gtgaaaagtt ttgtcaatgt agttcagagt gtcaaaaccg |
| 1861 | ctttccggga tgccgctgca aagcacagtg caacaccaag cagtgcccgt gctacctggc |
| 1921 | tgtccgagag tgtgaccctg acctctgtct tacttgtgga gccgctgacc attgggacag |
| 1981 | taaaaatgtg tcctgcaaga actgcagtat tcagcggggc tccaaaaagc atctattgct |
| 2041 | ggcaccatct gacgtggcag gctgggggat ttttatcaaa gatcctgtgc agaaaaatga |
| 2101 | attcatctca gaatactgtg gagagattat ttctcaagat gaagctgaca gaagagggaa |
| 2161 | agtgtatgat aaatacatgt gcagctttct gttcaacttg aacaatgatt ttgtggtgga |
| 2221 | tgcaacccgc aagggtaaca aaattcgttt tgcaaatcat tcggtaaatc caaactgcta |
| 2281 | tgcaaaagtt atgatggtta acggtgatca caggataggt atiatttgcca agagagccat |
| 2341 | ccagactggc gaagagctgt tttttgatta cagatacagc caggctgatg ccctgaagta |
| 2401 | tgtcggcatc gaaagagaaa tggaaatccc ttgacatctg ctacctcctc ccccctcctc |
| 2461 | tgaaacagct gccttagctt caggaacctc gagtactgtg ggcaatttag aaaaagaaca |
| 2521 | tgcagtttga aattctgaat ttgcaaagta ctgtaagaat aatttatagt aatgagttta |
| 2581 | aaaatcaact ttttattgcc ttctcaccag ctgcaaagtg ttttgtacca gtgaattttt |
| 2641 | gcaataatgc agtatggtac atttttcaac tttgaataaa gaatacttga acttgtcctt |
| 2701 | gttgaatc |
| (SEQ ID NO: 14) NP_001190176 histone-lysine N-methyltransferase EZH2 isoform c | |
| [Homo sapiens] | |
| 1 | mgqtgkksek gpvcwrkrvk seymrlrqlk rfrradevks mfssnrqkil erteilnqew |
| 61 | kqrriqpvhi ltsvsslrgt recsvtsdld fptqviplkt lnavasvpim yswsplqqnf |
| 121 | mvedetvlhn ipymgdevld qdgtfieeli knydgkvhgd recgfindei fvelvnalgq |
| 181 | yndddddddg ddpeereekq kdledhrddk esrpprkfps dkifeaissm fpdkgtaeel |
| 241 | kekykelteq qlpgalppec tpnidgpnak svqreqslhs fhtlfcrrcf kydcflhpfh |
| 301 | atpntykrkn tetaldnkpc gpacyghleg akefaaalta eriktppkrp ggrrrgrlpn |
| 361 | nssrpstpti nvleskdtds dreagtetgg enndkeeeek kdetssssea nsrcqtpikm |
| 421 | kpnieppenv ewsgaeasmf rvligtyydn fcaiarligt ktcrqvyefr vkessiiapa |
| 481 | paedvdtppr kkkrkhrlwa ahcrkiqikk dgssnhvyny qpcdhprqpc dsscpcviaq |
| 54i | nfcekfcqcs secqnrfpgc rckaqcntkq cpcylavrec dpdlcltcga adhwdsknvs |
| 601 | ckncsiqrgs kkhlllapsd vagwgifikd pvqknefise ycgeiisqde adrrgkvydk |
| 661 | ymcsflfnln ndfvvdatrk gnkirfanhs vnpncyakvm mvngdhrigi fakraiqtge |
| 721 | elffdyrysq adalkyvgie remeip |
| (SEQ ID NO: 15) NM_001195427 Homo sapiens serine/arginine-rich splicing factor 2 | |
| (SRSF2), transcript variant 2, mRNA | |
| 1 | agaaggtttc atttccgggt ggcgcgggcg ccattttgtg aggagcgata taaacgggcg |
| 61 | cagaggccgg ctgcccgccc agttgttact caggtgcgct agcctgcgga gcccgtccgt |
| 121 | gctgttctgc ggcaaggcct ttcccagtgt ccccacgcgg aaggcaactg cctgagaggc |
| 181 | gcggcgtcgc accgcccaga gctgaggaag ccggcgccag ttcgcggggc tccgggccgc |
| 241 | cactcagagc tatgagctac ggccgccccc ctcccgatgt ggagggtatg acctccctca |
| 301 | aggtggacaa cctgacctac cgcacctcgc ccgacacgct gaggcgcgtc ttcgagaagt |
| 361 | acgggcgcgt cggcgacgtg tacatcccgc gggaccgcta caccaaggag tcccgcggct |
| 421 | tcgccttcgt tcgctttcac gacaagcgcg acgctgagga cgctatggat gccatggacg |
| 481 | gggccgtgct ggacggccgc gaactgcggg tgcaaatggc gcgctacggc cgcaccccgg |
| 541 | actcacacca cagccgccgg ggaccgccac cccgcaggta cgggggcggt ggctacggac |
| 601 | gccggagccg cagccctagg cggcgtcgcc gcagccgatc ccggagtcgg agccgttcca |
| 661 | ggtctcccag ccgatctcgc tacagccgct cgaagtctcg gtcccgcact ccttctcgat |
| 721 | ctcggtcgac ctccaagtcc agatccgcac gaaggtccaa gtccaagtcc tcgtcggtct |
| 781 | ccagatctcg ttcgcggtcc aggtcccggt ctcggtccag gagtcctccc ccagtgtcca |
| 841 | agagggaatc caaatccagg tcgcgatcga agagtccccc caagtctcct gaagaggaag |
| 901 | gagcggtgtc ctcttaagaa aatgatgtat cggcaagcag tgtaaacgga ggacttgggg |
| 961 | aaaaaggacc acatagtcca tcgaagaaga gtccttggaa caagcaactg gctattgaaa |
| 1021 | aggttatttt gtaacatttg tctaactttt tacttgttta agctttgcct cagttggcaa |
| 1081 | acttcatttt atgtgccatt ttgttgctgt tattcaaatt tcttgtaatt tagtgaggtg |
| 1141 | aacgacttca gatttcatta ttggatttgg atatttgagg taaaatttca ttttgttata |
| 1201 | tagtgctgac tttttttgtt tgaaattaaa cagattggta acctaatttg tggcctcctg |
| 1261 | acttttaagg aaaacgtgtg cagccattac acacagccta aagctgtcaa gagattgact |
| 1321 | cggcattgcc ttcattcctt aaaattaaaa acctacaaaa gttggtgtaa atttgtatat |
| 1381 | gttatttacc ttcagatcta aatggtaatc tgaacccaaa tttgtataaa gacttttcag |
| 1441 | gtgaaaagac ttgatttttt gaaaggattg tttatcaaac acaattctaa tctcttctct |
| 1501 | tatgtatttt tgtgcactag gcgcagttgt gtagcagttg agtaatgctg gttagctgtt |
| 1561 | aaggtggcgt gttgcagtgc agagtgcttg gctgtttcct gttttctccc gattgctcct |
| 1621 | gtgtaaagat gccttgtcgt gcagaaacaa atggctgtcc agtttattaa aatgcctgac |
| 1681 | aactgcactt ccagtcaccc gggccttgca tataaataac ggagcataca gtgagcacat |
| 1741 | ctagctgatg ataaatacac ctttttttcc ctcttccccc taaaaatggt aaatctgatc |
| 1801 | atatctacat gtatgaactt aacatggaaa atgttaagga agcaaatggt tgtaactttg |
| 1861 | taagtactta taacatggtg tatctttttg cttatgaata ttctgtatta taaccattgt |
| 1921 | ttctgtagtt taattaaaac attttcttgg tgttagcttt tctcagaaaa aaaaaaaaaa |
| 1981 | aaaaaaaaaa aaaaaaaaaa aaaaaaaa |
| (SEQ ID NO: 16) NP_001182356 serine/arginine-rich splicing factor 2 [Homo sapiens] | |
| 1 | msygrpppdv egmtslkvdn ltyrtspdtl rrvfekygrv gdvyiprdry tkesrgfafv |
| 61 | rfhdkrdaed amdamdgavl dgrelrvqma rygrppdshh srrgppprry ggggygrrsr |
| 121 | sprrrrrsrs rsrsrsrsrs rsrysrsksr srtrsrsrst sksrsarrsk sksssysrsr |
| 181 | srsrsrsrsr spppvskres ksrsrskspp kspeeegavs s |
| (SEQ ID NO: 17) NM_005896 Homo sapiens isocitrate dehydrogenase 1 (NADP+), soluble | |
| (IDH1), transcript variant 1, mRNA | |
| 1 | gggctgagga ggcggggcct gggaggggac aaagccggga agaggaaaag ctcggaccta |
| 61 | ccctgtggtc ccgggtttct gcagagtcta cttcagaagc ggaggcactg ggagtccggt |
| 121 | ttgggattgc caggctgtgg ttgtgagtct gagcttgtga gcggctgtgg cgccccaact |
| 181 | cttcgccagc atatcatccc ggcaggcgat aaactacatt cagttgagtc tgcaagactg |
| 241 | ggaggaactg gggtgataag aeatctattc actgtcaagg tttattgaag tcaaaatgtc |
| 301 | caaaaaaatc agtggcggtt ctgtggtaga gatgcaagga gatgaaatga cacgaatcat |
| 361 | ttgggaattg attaaagaga aactcatttt tccctacgtg gaattggatc tacatagcta |
| 421 | tgatttaggc atagagaatc gtgatgccac caacgaccaa gtcaccaagg atgctgcaga |
| 481 | agctataaag aagcataatg ttggcgtcaa atgtgccact atcactcctg atgagaagag |
| 541 | ggttgaggag ttcaagttga aacaaatgtg gaaatcacca aatggcacca tacgaaatat |
| 601 | tctgggtggc acggtcttca gagaagccat tatctgcaaa aatatccccc ggcttgtgag |
| 661 | tggatgggta aaacctatca tcataggtcg tcatgcttat ggggatcaat acagagcaac |
| 721 | tgattttgtt gttcctgggc ctggaaaagt agagataacc tacacaccaa gtgacggaac |
| 781 | ccaaaaggtg acatacctgg tacataactt tgaagaaggt ggtggtgttg ccatggggat |
| 841 | gtataatcaa gataagtcaa ttgaagattt tgcacacagt tccttccaaa tggctctgtc |
| 901 | taagggttgg cctttgtatc tgagcaccaa aaacactatt ctgaagaaat atgatgggcg |
| 961 | ttttaaagac atctttcagg agatatatga caagcagtac aagtcccagt ttgaagctca |
| 1021 | aaagatctgg tatgagcata ggctcatcga cgacatggtg gcccaagcta tgaaatcaga |
| 1081 | gggaggcttc atctgggcct gtaaaaacta tgatggtgac gtgcagtcgg actctgtggc |
| 1141 | ccaagggtat ggctctctcg gcatgatgac cagcgtgctg gtttgtccag atggcaagac |
| 1201 | agtagaagca gaggctgccc acgggactgt aacccgtcac taccgcatgt accagaaagg |
| 1261 | acaggagacg tccaccaatc ccattgcttc catttttgcc tggaccagag ggttagccca |
| 1321 | cagagcaaag cttgataaca ataaagagct tgccttcttt gcaaatgctt tggaagaagt |
| 1381 | ctctattgag acaattgagg ctggcttcat gaccaaggac ttggctgctt gcattaaagg |
| 1441 | tttacccaat gtgcaacgtt ctgactactt gaatacattt gagttcatgg ataaacttgg |
| 1501 | agaaaacttg aagatcaaac tagctcaggc caaactttaa gttcatacct gagctaagaa |
| 1561 | ggataattgt cttttggtaa ctaggtctac aggtttacat ttttctgtgt tacactcaag |
| 1621 | gataaaggca aaatcaattt tgtaatttgt ttagaagcca gagtttatct tttctataag |
| 1681 | tttacagcct ttttcttata tatacagtta ttgccacctt tgtgaacatg gcaagggact |
| 1741 | tttttacaat ttttatttta ttttctagta ccagcctagg aattcggtta gtactcattt |
| 1301 | gtattcactg tcactttttc tcatgttcta attataaatg accaaaatca agattgctca |
| 1861 | aaagggtaaa tgatagccac agtattgctc cctaaaatat gcataaagta gaaattcact |
| 1921 | gccttcccct cctgtccatg accttgggca cagggaagtt ctggtgtcat agatatcccg |
| 1981 | ttttgtgagg tagagctgtg cattaaactt gcacatgact ggaacgaagt atgagtgcaa |
| 2041 | ctcaaatgtg ttgaagatac tgcagtcatt tttgtaaaga ccttgctgaa tgtttccaat |
| 2101 | agactaaata ctgtttaggc cgcaggagag tttggaatcc ggaataaata ctacctggag |
| 2161 | gtttgtcctc tccatttttc tctttctcct cctggcctgg cctgaatatt atactactct |
| 2221 | aaatagcata tttcatccaa gtgcaateat gtaagctgaa tcttttttgg acttctgctg |
| 2281 | gcctgtttta tttcttttat ataaatgtga tttctcagaa attgatatta aacactatct |
| 2341 | tatcttctcc tgaactgttg attttaatta aaattaagtg ctaattacca ttaaaaaaaa |
| 2401 | aa |
| (SEQ ID NO: 18) NP_005887 isocitrate dehydrogenase [NADP] cytoplasmic [Homo sapiens] | |
| 1 | mskkisggsv vemqgdemtr iiwelikekl ifpyveldlh sydlgienrd atndqvtkda |
| 61 | aeaikkhnvg vkcatitpde krveefklkq mwkspngtir nilggtvfre aiickniprl |
| 121 | vsgwvkpiii grhaygdqyr atdfvvpgpg kveitytpsd gtqkvtylvh nfeegggvam |
| 181 | gmynqdksie dfahssfqma lskgwplyls tkntilkkyd grfkdifqei ydkqyksqfe |
| 241 | aqkiwyehrl iddmvaqamk seggfiwack nydgdvqsds vaqgygslgm mtsvlvcpdg |
| 301 | ktveaeaahg tvtrhyrmyq kgqetstnpi asifawtrgl ahrakldnnk elaffanale |
| 361 | evsietieag fmtkdlaaci kglpnvqrsd ylntfefmdk lgenlkikla qakl |
| (SEQ ID NO: 19) NM_001289910 Homo sapiens isocitrate dehydrogenase 2 (NADP+), | |
| mitochondrial (IDH2), transcript variant 2, mRNA | |
| 1 | attttgtaac gccataggct tccagcgact gctggtgatg tttctgatgc cgacaaaagg |
| 61 | atcaaggtgg cgaaggccgt ggtggagatg gatggtgatg agatgacccg tattatctgg |
| 121 | cagttcatca aggagaagct catcctgccc cacgtggaca tccagctaaa gtattttgac |
| 111 | ctcgggctcc caaaccgtga ccagactgat gaccaggtca ccattgactc tgcactggcc |
| 241 | acccagaagt acagtgtggc tgtcaagtgt gccaccatca cccctgatga ggcccgtgtg |
| 301 | gaagagttca agctgaagaa gatgtggaaa agtcccaatg gaactatccg gaacatcctg |
| 361 | ggggggactg tattccggga gcccatcatc tgcaaaaaca tcccacgcct agtccctggc |
| 421 | tggaccaagc ccatcaccat tggcaggcac gcccatggcg accagtacaa ggccacagac |
| 481 | tttgtggcag accgggccgg cactttcaaa atggtcttca ccccaaaaga tggcagtggt |
| 541 | gtcaaggagt gggacgtgta caacttcccc gcaggcggcg tgggcatggg catgtacaac |
| 601 | accgacgagt ccatctcagc ttttgcgcac agctgcttcc agtatgccat ccagaagcaa |
| 661 | tggccgctgt acatgagcac caagaacacc atactgaaag cctacgatgg gcgtttcaag |
| 721 | gacatcttcc aggagatctt tgacaagcag tataagaccg agttcgacaa gaataagatc |
| 781 | tggtatgagc accggctcat tgatgacatg gtggctcagg tcctcaagtc ttcgggtggc |
| 841 | tttgtgtggg cctgcaagaa ctatgacgca gatgtgcagt cagacatcct ggcccagggc |
| 901 | tttggctccc ttggcctgat gacgtccgtc ctggtctgcc ctgatgggaa gacgattgag |
| 961 | gctgaggccg ctcatgggac cgtcacccgc cactatcggg agcaccagaa gggccggccc |
| 1021 | accagcacca accccatcgc cagcatcttt gcctggacac gtggcctgga gcacgggcgg |
| 1081 | aagctggatg ggaaccaaga cctcatcagg tttgcccaga tgctggagac ggtgtgcgtg |
| 1141 | gagacggtgc agagtgctac catgaccaag gacctggcgg gctgcattca cggcctcagc |
| 1201 | aatgtgaagc tgaacgagca cttcctgaac accacggcct tcctcgacac catcaagagc |
| 1261 | aacctggaca gagccctggg caggcagtag ggggaggcgc cacccatggc tgcagtggag |
| 1321 | gggccagggc tgagccggcg ggtcctcctg agcgcggcag agggtgaggc tcacagcccc |
| 1381 | tctctggagg cctttctagg ggatgttttt ttataagcca gatgttttta aaagcatatg |
| 1441 | tgtutttccc ctcatggtga cgtgaggcag gagcattgcg ttttacctca gccagtcagt |
| 1501 | atgttttgca tactgtaatt tatattgccc ttggaacaca tggtgccata tttagctact |
| 1561 | aaaaaggtct tcacaaaa |
| (SEQ ID NO: 20) NP_001276839 isocitrate dehydrogenase [NADP], mitochondrial isoform | |
| 2 [Homo sapiens] | |
| 1 | mdgdemtrii wqfikeklil phvdiqlkyf dlglpnrdqt ddqvtidsal atqkysvavk |
| 61 | catitpdear veefklkkmw kspngtirni lggtvfrepi ickniprlvp gwtkpitigr |
| 121 | hahgdqykat dfvadragtf kmvftpkdgs gvkewevynf paggvgmgmy ntdesisgfa |
| 181 | hscfqyaiqk kwplymstkn tilkaydgrf kdifqeifdk hyktdfdknk iwyehrlidd |
| 241 | mvaqvlkssg gfvwacknyd gdvqsdilaq gfgslglmts vlvcpdgkti eaeaahgtvt |
| 301 | rhyrehqkgr ptstnpiasi fawtrglehr gkldgnqdli rfaqmlekvc vetvesgamt |
| 361 | kdlagcihgl snvklnehfl nttdfldtik snldralgrq |

1. A method for treating a myeloproliferative disorder, comprising:
(i) determining whether the cells of a subject having a myeloproliferative disorder comprise a mutation associated with the myeloproliferative disorder in one or more genes selected from: JAK2, MPL, CALR, ASXL1, EZH2, SRSF2, IDH1, or IDH2; and
if the subject comprises said mutant allele
(ii) administering an effective amount of a serum amyloid P (SAP) protein to the subject.

2. The method of claim 1, wherein the myeloproliferative disorder is primary myelofibrosis, post-polycythemia vera myelofibrosis, or post-essential thrombocythemia myelofibrosis.

3-4. (canceled)

5. The method of claim 1, wherein the method comprises determining whether the subject comprises a mutation selected from the group consisting of a mutation at codon 617 of JAK2, a mutation in exon 12 or exon 14 of JAK2, a mutation at codon 515 of MPL, a mutation in exon 10 of MPL, a mutation in exon 9 of CALR, a mutation in exon 12 of ASXL1, a mutation in exon 4 of IDH1, a mutation at codon 132 of IDH1, a mutation in exon 4 of IDH2, a mutation at codon 140 of IDH2, and a mutation at codon 172 of IDH2.

6.-20. (canceled)

21. A method for treating a myeloproliferative disorder with a serum amyloid P (SAP) protein, the method comprising:
(i) measuring a first mutant allele burden of a mutation in one or more genes associated with the myeloproliferative disorder selected from: JAK2, MPL, CALR, ASXL1, EZH2, SRSF2, IDH1, or IDH2, wherein said first mutant allele burden is measured before administration of the SAP protein;
(ii) measuring a second mutant allele burden of the same mutation measured in (i), wherein said second mutant allele burden is measured after administration of the SAP protein; and
(iii) identifying a difference between the second mutant allele burden and the first mutant allele burden,
wherein a decrease in the second mutant allele burden relative to the first mutant allele burden indicates that the administration of the SAP protein is effective in treating the myeloproliferative disorder.

22. The method of claim 21, wherein the decrease in the second mutant allele burden relative to the first mutant allele burden is 10% to 90% or the difference between the second mutant allele burden and the first mutant allele burden is a reduction by 10% to 90%.

23.-24. (canceled)

25. The method of claim 21, wherein the difference is a complete molecular response.

26.-27. (canceled)

28. The method of claim 21, wherein the measuring step comprises amplifying nucleic acid comprising all or a portion of the one or more genes associated with the myeloproliferative disorder.

29. (canceled)

30. The method of claim 21, further comprising increasing, decreasing, or maintaining the dosage regimen of the SAP protein based on the effectiveness of the treatment.

31. The method of claim 21, wherein at least one of the mutations in one or more genes is selected from the group consisting of a mutation at codon 617 of JAK2, a mutation in exon 12 or exon 14 of JAK2, a mutation at codon 515 of MPL, a mutation in exon 10 of MPL, a mutation in exon 9 of CALR a mutation in exon 12 of ASXL1, a mutation in exon 4 of IDH1, a mutation at codon 132 of IDH1, a mutation in exon 4 of IDH2, a mutation at codon 140 of IDH2, and a mutation at codon 172 of IDH2.

32.-47. (canceled)

48. A method of treating a myeloproliferative disorder, comprising administering to a subject in need thereof an effective amount of a serum amyloid P (SAP) protein, wherein some of the subject's cells comprise a mutation associated with the myeloproliferative disorder in one or more genes selected from: JAK2, MPL, CALR, ASXL1, EZH2, SRSF2, IDH1, or IDH2, and wherein the SAP protein is administered according to a dosage regimen effective to reduce mutant allele burden of said gene in said subject.

49. The method of claim 48, wherein the myeloproliferative disorder comprises primary myelofibrosis, post-polycythemia vera myelofibrosis, or post-essential thrombocythemia myelofibrosis.

50.-51. (canceled)

52. The method of claim 48, wherein the subject comprises a mutation selected from the group consisting of a mutation at codon 617 of JAK2, a mutation in exon 12 or exon 14 of JAK2, a mutation at codon 515 of MPL, a mutation in exon 10 of MPL, a mutation in exon 9 of CALR, a mutation in exon 12 of ASXL1, a mutation in exon 4 of IDH1, a mutation at codon 132 of IDH1, a mutation in exon 4 of IDH2, a mutation at codon 140 of IDH2, and a mutation at codon 172 of IDH2.

53.-70. (canceled)

71. The method of claim 1, wherein the SAP protein comprises an SAP polypeptide comprising an amino acid sequence at least 85% identical to SEQ ID NO: 1.

72. The method of claim 1, wherein the SAP protein comprises a glycosylated recombinant human SAP polypeptide comprising an N-linked oligosaccharide chain, wherein at least one branch of the oligosaccharide chain terminates with a α2,3-linked sialic acid moiety.

73. The method of claim 72, wherein all sialylated branches of the oligosaccharide chain terminate with α2,3-linked sialic acid moieties.

74. The method of claim 72, wherein the oligosaccharide chain is substantially free of α2,6-linked sialic acid moieties.

75.-78. (canceled)

79. The method of claim 1, wherein the polypeptide comprises one or more modified amino acid residues selected from the group consisting of a PEGylated amino acid, a prenylated amino acid, an acetylated amino acid, a biotinylated amino acid, and an amino acid conjugated to an organic derivatizing agent.

80.-81. (canceled)

82. The method of claim 1, wherein the method further comprises administering to the patient an additional anti-cancer therapeutic.

83.-90. (canceled)

91. The method of claim 1, wherein the SAP protein is administered according to a dosage regimen effective to reduce spleen volume by at least 25% relative to baseline.

92. (canceled)

93.-104. (canceled)
